(19) United States
(12) Patent Application Publication
(10) Pub. No.: US 2024/0228495 A1
(43) Pub. Date: Jul. 11, 2024

(54) Tricyclic Inhibitors of Influenza Virus Endonuclease

(71) Applicant: SRI INTERNATIONAL, Menlo Park, CA (US)

(72) Inventors: Thomas R. Webb, Mountain View, CA (US); Chandraiah Lagisetti, Cupertino, CA (US); Diane Beylkin, Thousand Oaks, CA (US); Jaehyeon Park, Sunnyvale, CA (US); Wei Zhou, Fremont, CA (US); Peter Madrid, San Jose, CA (US); Leyi Gong, San Mateo, CA (US); Jeremiah Malerich, San Jose, CA (US); Chat Gheong Gabriel Fung, San Francisco, CA (US); Raymond Ng, Pleasant Hill, CA (US); Quentin Perron, Evecquemont (FR); Vinicius Barros Ribeiro da Silva, Paris (FR)

(21) Appl. No.: 17/913,824

(22) PCT Filed: Mar. 24, 2021
(86) PCT No.: PCT/US2021/023980

§ 371 Date: Sep. 22, 2022

Related U.S. Application Data
(60) Provisional application No. 62/994,535, filed on Mar. 25, 2020.

(51) Int. Cl. C07D 487/04; A61K 31/519; A61K 31/551; A61K 45/06

(52) U.S. Cl. CPC C07D 487/04; A61K 31/519; A61K 31/551; A61K 45/06

(57) ABSTRACT

The present disclosure is concerned with 9-hydroxy-6-(pyrrolidin-2-yl)-3, 4-dihydro-2H-pyrazino[1,2-c]pyrimidine-1,8-dione compounds for the treatment of various viral infections such as, for example, influenza virus. This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/994,535, filed on Mar. 25, 2020, the contents of which are hereby incorporated by reference in their entirety.

### STATEMENT REGARDING FEDERALLY SPONSORED RESEARCH

This invention was made with government support under grant number R01AI098757 awarded by the National Institute of Allergy and Infectious Diseases of the National Institutes of Health. The government has certain rights in the invention.

### BACKGROUND

Influenza is a serious threat to human health recent years. Indeed, the world has experienced several widespread outbreaks of this respiratory disease, including an epidemic of H3N2 influenza in 2017-2018 that unable to be well-addressed through the available vaccines. 2018 was the 100^{th} anniversary of the 1918 “Spanish flu” pandemic, which is estimated to have caused between 20 and 40 million people deaths in just eight months. Major influenza pandemics in the 20^{th} century include the 1957 “Asian flu,” and the 1968 “Hong Kong flu,” with each resulting in approximately 1 million deaths.

One approach to rapidly addressing pandemics is to develop small molecule antivirals that have broad activity against all strains of influenza. The majority of small molecule anti-influenza drugs that are currently on the market are neuraminidase inhibitors (e.g., zanamivir, oseltamivir, peramivir) or target the M2-ion channel (e.g., amantadine, rimantadine). Unfortunately, these targets, particularly the latter, are prone to rapid mutations that can confer antiviral resistance due to the inability of the viral RNA dependent RNA polymerase (RdRp) to proofread during RNA replication. In fact, the World Health Organization's Global Influenza Program reported that >99% of seasonal influenza A strains are now resistant to amantadine and rimantadine. This has led to the search for new antiviral compounds that target other essential viral processes. Exemplary compounds that are currently in clinical trials include Favipiravir, VX-787 (now known as JNJ63623872), Fludase, Nitazoxanide, and S033188. Despite these efforts, however, no new influenza drugs are currently available to the general population. Thus, there remains a need for compounds and compositions for treating influenza, and methods of making and using same.

### SUMMARY

In accordance with the purpose(s) of the invention, as embodied and broadly described herein, the invention, in one aspect, relates to compounds and compositions for use in the prevention and treamtnent of viral infections due to, for example, an Alphavirus (e.g., Chikungunya virus (CHIKV), Ross River virus, Venezuelan equine encephalitis (VEEV), Eastern equine encephalitis (EEEV), and Western equine encephalitis (WEEV)), a Flavivirus (e.g., dengue virus (DENV), West Nile virus (WNV), zika virus (ZIKV), tick-borne encephalitis virus, and yellow fever virus), Coronavirus (e.g., Middle East Respiratory Syndromes coronavirus (MERS-CoV), Severe Acute Respiratory Syndrome coronavirus (SARS-CoV), and SARS-CoV-2), and influenza viruses (influenza A and influenza B).

Thus, disclosed are compounds having a structure represented by a formula:

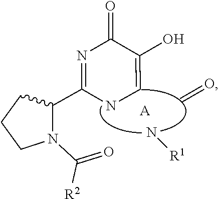

wherein: A is a 6-7 membered heterocycle; R¹ is C1-C3 alkyl, C1-C3 alkoxy, C1-C3 haloalkyl, C1-C3 alkylsulfonyl, or a 9- to 10-membered cycloaryl, wherein R¹ can further be independently substituted with one or more R^{x} groups; R^{x} is sulfonyl, oxo, C1-C2 haloalkyl, 5- to 6-membered aryl, or 5- to 6-membered heteroaryl, wherein R^{x} can independently further be substituted with one or more R^{a} groups; R^{a} is C1-C2 alkyl, 5- to 6-membered aryl, 5- to 6-membered heteroaryl, or C1-C2 haloalkyl, wherein R^{a} can independently be substituted with one or more R^{a1} groups; R^{a1} is halo, C1-C2 alkoxy, cyano, or C1-C2 haloalkyl; R² is C1-C2 alkyl, 5- to 6-membered aryl, oxo, 5- to 6-membered heteroaryl, or C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further be substituted with one or more R^{b} groups; R^{b} is halo or 5- to 6-membered aryl; R^{b1} is halo; and wherein the wavy line indicates either R or S enantiomer at that bond, or a pharmaceutically acceptable salt or hydrate thereof.

Also disclosed are compounds having a structure represented by a formula:

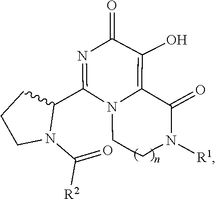

wherein: n is 1 or 2; R¹ is C1-C3 alkyl, C1-C3 haloalkyl, —(C1-C3 alkyl)OR¹⁰, —(C1-C3 alkyl)SO₂R¹⁰, or Cy¹; R¹⁰ is C1-C2 alkyl or Ar¹; Ar¹ is a 5- to 6-membered aryl or a 5- to 6-membered heteroaryl, and is substituted with 0, 1, 2, or 3 groups independently selected from halogen, —CN, C1-C2 alkyl, C1-C2 haloalkyl, and —C1-C2 alkoxy; Cy¹ is an unsubstituted 9- to 10-membered cycloalkyl group; R² is C1-C2 alkyl, —(C1-C2 alkyl)Ar², —O(C1-C2 alkyl), —O(C1-C2 alkyl)Ar², —(C1-C2 alkyl)OAr², —S(C1-C2 alkyl), —S(C1-C2 alkyl)Ar², —(C1-C2 alkyl)SAr², or Ar²; and Ar² is a 5- to 6-membered aryl or a 5- to 6-membered heteroaryl, and is substituted with 0, 1, 2, or 3 groups independently selected from halogen, —CN, C1-C2 alkyl, and C1-C2 haloalkyl, or a pharmaceutically acceptable salt thereof.

Also disclosed are pharmaceutical compositions comprising a therapeutically effective amount of a disclosed compound, and a pharmaceutically acceptable carrier.

Also disclosed are methods of treating a viral infection in a subject in need thereof, the method comprising administering to the subject an effective amount of a disclosed compound.

Also disclosed are methods of making a disclosed compound.

Also disclosed are kits comprising a disclosed compound, and one or more of: (a) an antiviral agent; (b) an immunity booster; (c) instructions for administering the compound in connection with treating a viral infection; (d) instructions for administering the compound in connection with reducing the risk of viral infection; and (e) instructions for treating a viral infection.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DETAILED DESCRIPTION

This disclosure describes inventive concepts with reference to specific examples. However, the intent is to cover all modifications, equivalents, and alternatives of the inventive concepts that are consistent with this disclosure. It will be apparent, however, to one of ordinary skill in the art that the present approach can be practiced without these specific details. Thus, the specific details set forth are merely exemplary, and is not intended to limit what is presently disclosed. The features implemented in one aspect may be implemented in another aspect where logically possible. The specific details can be varied from and still be contemplated to be within the spirit and scope of what is being disclosed.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used herein and in the appended claims, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated+10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The prefix “C_{X-Y}” indicates that the following group has from x (e.g., 1) to y (e.g., 6) carbon atoms, one or more of which, in certain groups (e.g., heteroalkyl, heteroaryl, heteroarylalkyl, etc.), may be replaced with one or more heteroatoms or heteroatomic groups. For example, “C₁₋₆ alkyl” indicates that the alkyl group has from 1 to 6 carbon atoms. Likewise, the term “x-y membered” rings, wherein x and y are numerical ranges, such as “3-12 membered heterocyclyl”, refers to a ring containing x-y atoms (e.g., 3-12), of which up to half may be heteroatoms, such as N, O, S, P, and the remaining atoms are carbon. Also, certain commonly used alternative chemical names may or may not be used. For example, a divalent group such as a divalent “alkyl” group, a divalent “aryl” group, etc., may also be referred to as an “alkylene” group or an “alkylenyl” group, or alkylyl group, an “arylene” group or an “arylenyl” group, or arylyl group, respectively.

“Alkyl” refers to any group derived from a linear or branched saturated hydrocarbon. Alkyl groups include, but are not limited to, methyl, ethyl, propyl such as propan-1-yl, propan-2-yl (iso-propyl), butyls such as butan-1-yl, butan-2-yl (sec-butyl), 2-methyl-propan-1-yl (iso-butyl), 2-methyl-propan-2-yl (t-butyl), pentyls, hexyls, octyls, dectyls, and the like. Unless otherwise specified, an alkyl group has from 1 to 10 carbon atoms, for example from 1 to 6 carbon atoms, for example from 1 to 4 carbon atoms.

“Alkenyl” refers to any group derived from a straight or branched hydrocarbon with at least one carbon-carbon double bond. Alkenyl groups include, but are not limited to, ethenyl (vinyl), propenyl (allyl), 1-butenyl, 1,3-butadienyl, and the like. Unless otherwise specified, an alkenyl group has from 2 to 10 carbon atoms, for example from 2 to 6 carbon atoms, for example from 2 to 4 carbon atoms.

“Alkynyl” refers to any group derived from a straight or branched hydrocarbon with at least one carbon-carbon triple bond and includes those groups having one triple bond and one double bond. Examples of alkynyl groups include, but are not limited to, ethynyl (—CH—CH), propargyl (—CH₂C≡CH), (E)-pent-3-en-1-ynyl, and the like. Unless otherwise specified, an alkynyl group has from 2 to 10 carbon atoms, for example from 2 to 6 carbon atoms, for example from 2 to 4 carbon atoms.

“Amino” refers to —NH2. Amino groups may also be substituted as described herein, such as with alkyl, carbonyl or other amino groups. The term “alkylamino” refers to an amino group substituted with one or two alkyl substituents (e.g. dimethylamino or propylamino).

“Aryl” refers to any group derived from one or more aromatic rings, that is, a single aromatic ring, a bicyclic or a multicyclic ring system. Aryl groups include, but are not limited to, those groups derived from acenaphthylene, anthracene, azulene, benzene, chrysene, a cyclopentadienyl anion, naphthalene, fluoranthene, fluorene, indane, perylene, phenalene, phenanthrene, pyrene and the like.

“Arylalkyl” (also “aralkyl”) refers to any combination aryl group and an alkyl group. Arylalkyl groups include, but are not limited to, those groups derived from benzyl, tolyl, dimethylphenyl, 2-phenylethan-1-yl, 2-naphthylmethyl, and the like. An arylalkyl group comprises from 6 to 30 carbon atoms, for example the alkyl group can comprise from 1 to 10 carbon atoms and the aryl group can comprise from 5 to 20 carbon atoms.

“Cycloaryl” refers to a combination an aryl group and a cyclic ring. Some representative example of a cycloaryl comprise 2,3-dihydro-1H-indene, 1,2,3,4-tetrahydronaphthalene, 3a,5,6,7-tetrahydro-4H-indene, and the like.

“Heterocyclo-aryl” refers to a combination of an aryl group and a heterocylic group. Some representative examples of a heterocyclo-aryl comprise 1,2,3,4-tetrahydroisoquinoline, isochromane, 1,3-dihydroisobenzofuran, isoindoline, and the like.

“Cycloalkyl” refers to a cyclic alkyl and alkenyl groups. A cycloalkyl group can have one or more cyclic rings and includes fused and bridged groups that are fully saturated or partially unsaturated. Examples include, but are not limited to, cyclopropyl, cyclobutyl, cyclopentyl, cyclohexyl, adamantyl, methylcycloproyl (cyclopropylmethyl), ethylcyclopropyl, cyclohexenyl and the like. Another example includes C₅-7 cycloakenyl.

“Halo” and “halogen” refer to fluoro, chloro, bromo and iodo.

“Haloalkyl” refers to an alkyl wherein one or more hydrogen atoms are each replaced by a halogen. Examples include, but are not limited to, —CH₂Cl, —CH₂F, —CH₂Br, —CFClBr, —CH₂CH₂Cl, —CH₂CH₂F, —CF₃, —CH₂CF₃, —CH₂CCl₃, and the like, as well as alkyl groups such as perfluoroalkyl in which all hydrogen atoms are replaced by fluorine atoms.

“Hydroxyalkyl” refers to an alkyl wherein one or more hydrogen atoms are each replaced by a hydroxyl group. Examples include, but are not limited to, —CH₂OH, —CH₂CH₂OH, —C(CH3)2OH, and the like.

“Halo 3-6 membered heterocyclyl” refers to a heterocyclyl group substituted at a carbon atom with at least one halogen atom, and may include multiple halogen atoms, such as 3,3-difluoroazetidinyl.

“Heteroalkyl” refers to an alkyl in which one or more of the carbon atoms (and any associated hydrogen atoms) are each independently replaced with the same or different heteroatom or heteroatomic group. Heteroatoms include, but are not limited to, N, P, O, S, etc. Heteroatomic groups include, but are not limited to, —NR, —O—, —S—, —PH—, —P(O)₂—, —S(O)—, —S(O)₂—, and the like, where R is H, alkyl, aryl, cycloalkyl, heteroalkyl, heteroaryl or cycloheteroalkyl. Heteroalkyl groups include, but are not limited to, —OCH₃, —CH₂OCH₃, —SCH₃, —CH₂SCH₃, —NRCH₃, —CH₂NRCH₃, —CH₂OH and the like, where R is hydrogen, alkyl, aryl, arylalkyl, heteroalkyl, or heteroaryl, each of which may be optionally substituted. A heteroalkyl group comprises from 1 to 10 carbon and up to three hetero atoms, e.g., from 1 to 6 carbon and from 1 to 2 hetero atoms.

“Heteroaryl” refers to mono or multicyclic aryl group in which one or more of the aromatic carbon atoms (and any associated hydrogen atoms) are independently replaced with the same or different heteroatom or heteroatomic group, as defined above. Multicyclic ring systems are included in heteroaryl and may be attached at the ring with the heteroatom or the aryl ring. Heteroaryl groups include, but are not limited to, groups derived from acridine, benzoimidazole, benzothiophene, benzofuran, benzoxazole, benzothiazole, carbazole, carboline, cinnoline, furan, imidazole, imidazopyridine, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Heteroaryl groups may have 5-14 members, 5-10 members, or 5-6 members.

“Heterocycle,” “heterocyclic,” and “heterocyclyl” refer to a saturated or partially unsaturated non-aromatic ring or a partially non-aromatic multiple-ring system with at least one heteroatom or heteroatomic group, as defined above. Heterocycles include, but are not limited to, groups derived from azetidine, aziridine, imidazolidine, morpholine, thiomorpholine, tetrahydro-2H-thiopyran, 1-iminotetrahydro-2H-thiopyran 1-oxide, oxirane (epoxide), oxetane, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-chloropiperidine, and the like. Heterocyclyl groups also include partially unsaturated ring systems containing one or more double bonds, including fused ring systems with one aromatic ring and one non-aromatic ring, but not fully aromatic ring systems. Examples include dihydroquinolines, e.g. 3,4-dihydroquinoline, dihydroisoquinolines, e.g. 1,2-dihydroisoquinoline, dihydroimidazole, tetrahydroimidazole, etc., indoline, isoindoline, isoindolones (e.g. isoindolin-1-one), isatin, dihydrophthalazine, quinolinone, spiro[cyclopropane-1,1′-isoindolin]-3′-one, and the like. Heterocycle groups may have 3-12 members, or 3-10 members, or 3-7 members, or 5-6 members.

“Hydroxyl” and “hydroxy” are used interchangeably and refer to —OH. “Oxo” refers to ═O, or oxide where N-oxide or S-oxide exist. Where tautomeric forms of the compound exist, hydroxyl and oxo groups are interchangeable.

It is understood that combinations of chemical groups may be used and will be recognized by persons of ordinary skill in the art. For instance, the group “hydroxyalkyl” would refer to a hydroxyl group attached to an alkyl group. A great number of such combinations may be readily envisaged. Additional examples of substituent combinations used herein include: C1-6 alkylamiocarbonyl (e.g. CH₃CH₂NHC(O)—) C₁₋₆ alkoxycarbonyl (e.g. CH₃O—C(O)—), 5-7 membered heterocyclyl-C1-6 alkyl (e.g. piperazinyl-CH₂—), C₁₋₆ alkylsulfonyl-5-7 membered heterocyclyl (e.g. CH₃S(O)₂-morpholinyl-), 5-7 membered heterocyclyl C₁₋₆ alkoxy (e.g. pyrrolidinyl-O—), 5-7 membered heterocyclyloxy, (4-7 membered heterocyclyl)-4-7 membered heterocyclyl (e.g. oxetanyl-pyrrolidinyl-), C₃₋₆ cycloalkylaminocarbonyl (e.g. cyclopropyl-NH— C(O)—), 5-7 membered heterocyclyl-C₂₋₆ alkynyl (e.g. N-piperazinyl-CH₂C≡CCH₂—), and C₆₋₁₀ arylaminocarbonyl (e.g. phenyl-NH—C(O)—).

“Sulfonyl,” as used herein, refers to —SO₂—.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

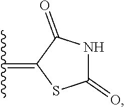 [SMILES: *=C1SC(=O)NC1=O]

regardless of whether thiazolidinedione is used to prepare the compound. In some aspects the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some aspects, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

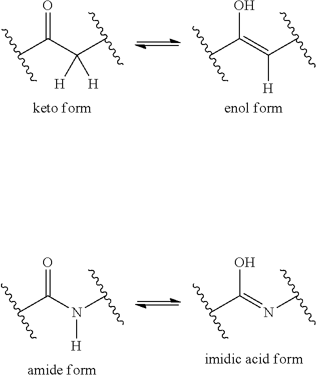 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N-unsubstituted, 5-A³ as shown below.

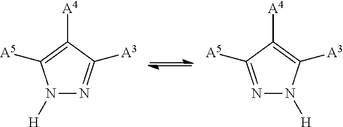

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

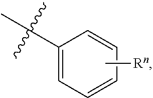

which is understood to be equivalent to a formula:

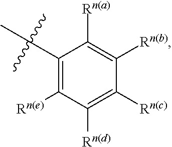

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, pocket pets, rabbits, dogs, and monkeys), and the like.

“Treating” and “treatment” of a disease include the following: (1) preventing or reducing the risk of developing the disease, i.e., causing the clinical symptoms of the disease not to develop in a subject that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

“Effective amount” refers to an amount that may be effective to elicit the desired biological, clinical, or medical response, including the amount of a compound that, when administered to a subject for treating a disease, is sufficient to effect such treatment. The effective amount will vary depending on the compound, the disease and its severity and the age, weight, etc., of the subject to be treated. The effective amount can include a range of amounts.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The compounds of the invention include solvates, hydrates, tautomers, stereoisomers and salt forms thereof.

The pharmaceutical compositions of compounds of the disclosed formulas may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. In one aspect, the compounds described herein may be administered orally. Oral administration may be via, for example, capsule or enteric coated tablets.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use.

Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful in treating disorders associated with a viral infection due to, in particular, an Alphavirus (e.g., Chikungunya virus (CHIKV), Ross River virus, Venezuelan equine encephalitis (VEEV), Eastern equine encephalitis (EEEV), and Western equine encephalitis (WEEV)), a Flavivirus (e.g., dengue virus (DENV), West Nile virus (WNV), zika virus (ZIKV), tick-bome encephalitis virus, and yellow fever virus), a Coronavirus (e.g., Middle East Respiratory Syndromes coronavirus (MERS-CoV), Severe Acute Respiratory Syndrome coronavirus (SARS-CoV), and SARS-CoV-2), or an influenza virus (influenza A and influenza B). In a further aspect, the viral infection is due to an influenza virus.

In one aspect, the disclosed compounds exhibit antiviral activity.

In one aspect, the compounds of the invention are useful in inhibiting viral activity in a mammal. In a further aspect, the compounds of the invention are useful in inhibiting viral activity in at least one cell.

In one aspect, the compounds of the invention are useful in the treatment of viral infections, as further described herein.

It is contemplated that each disclosed derivative can be optionally further substituted.

It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

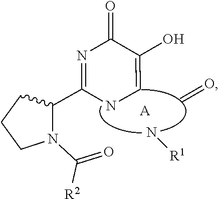

wherein: A is a 6-7 membered heterocycle; R¹ is C1-C3 alkyl, C1-C3 alkoxy, C1-C3 haloalkyl, C1-C3 alkylsulfonyl, or a 9- to 10-membered cycloaryl, wherein R¹ can further be independently substituted with one or more R^{x} groups; R^{x} is sulfonyl, oxo, C1-C2 haloalkyl, 5- to 6-membered aryl, or 5- to 6-membered heteroaryl, wherein R^{x} can independently further be substituted with one or more R^{a} groups; R^{a} is C1-C2 alkyl, 5- to 6-membered aryl, 5- to 6-membered heteroaryl, or C1-C2 haloalkyl, wherein R^{a} can independently be substituted with one or more R^{a1} groups; R^{a1} is halo, C1-C2 alkoxy, cyano, or C1-C2 haloalkyl; R² is C1-C2 alkyl, 5- to 6-membered aryl, oxo, 5- to 6-membered heteroaryl, or C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further be substituted with one or more R^{b} groups; R^{b} is halo or 5- to 6-membered aryl; R^{b1} is halo; and wherein the wavy line indicates either R or S enantiomer at that bond, or a pharmaceutically acceptable salt or hydrate thereof.

In a further aspect, disclosed are compounds having a structure represented by a formula:

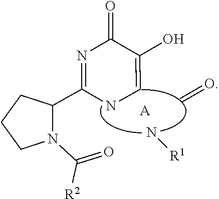

In a further aspect, disclosed are compounds having a structure represented by a formula selected from:

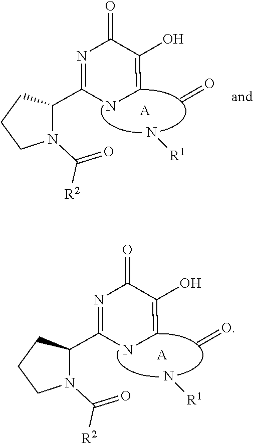

In one aspect, disclosed are compounds having a structure represented by a formula:

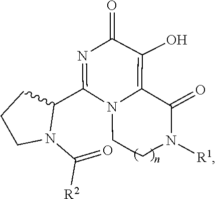

wherein: n is 1 or 2; R¹ is C1-C3 alkyl, C1-C3 haloalkyl, —(C1-C3 alkyl)OR¹⁰, —(C1-C3 alkyl)SO₂R¹⁰, or Cy¹; R¹⁰ is C1-C2 alkyl or Ar¹; Ar¹ is a 5- to 6-membered aryl or a 5- to 6-membered heteroaryl, and is substituted with 0, 1, 2, or 3 groups independently selected from halogen, —CN, C1-C2 alkyl, C1-C2 haloalkyl, and —C1-C2 alkoxy; Cy¹ is an unsubstituted 9- to 10-membered cycloalkyl group; R² is C1-C2 alkyl, —(C1-C2 alkyl)Ar², —O(C1-C2 alkyl), —O(C1-C2 alkyl)Ar², —(C1-C2 alkyl)OAr², —S(C1-C2 alkyl), —S(C1-C2 alkyl)Ar², —(C1-C2 alkyl)SAr², or Ar²; and Ar² is a 5- to 6-membered aryl or a 5- to 6-membered heteroaryl, and is substituted with 0, 1, 2, or 3 groups independently selected from halogen, —CN, C1-C2 alkyl, and C1-C2 haloalkyl, or a pharmaceutically acceptable salt thereof.

In a further aspect, disclosed are compounds having a structure represented by a formula:

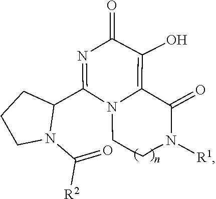

disclosed are compounds having a structure represented by a formula selected from:

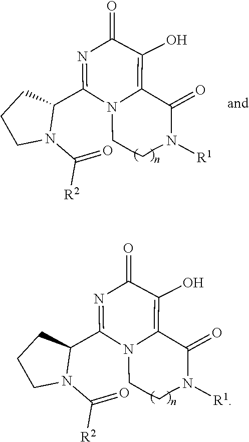

In some aspects, a compound of Formula IA is provided:

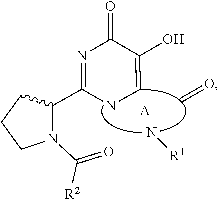

wherein: A is a 6-7 membered heterocycle; R¹ is C1-C3 alkyl, C1-C3 alkoxy, C1-C3 haloalkyl, C1-C3 alkylsulfonyl, or 9- to 10-membered cycloaryl, wherein R¹ can further be independently substituted with one or more R^{x} groups; R^{x} is sulfonyl, oxo, C1-C2 haloalkyl, 5- to 6-membered aryl, or 5- to 6-membered heteroaryl wherein R^{x} can independently further be substituted with one or more R^{a} groups; R^{a} is C1-C2 alkyl, 5- to 6-membered aryl, 5- to 6-membered heteroaryl, or C1-C2 haloalkyl, wherein R^{a} can independently be substituted with one or more R^{a1} groups; R^{a1} is halo, C1-C2 alkoxy, cyano, or C1-C2 haloalkyl; R² is C1-C2 alkyl, 5- to 6-membered aryl, oxo, 5- to 6-membered heteroaryl, or C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further substituted with one or more R^{b} groups; R^{b} is halo or 5- to 6-membered aryl; R^{b1} is halo; and wherein the wavy line indicates either R or S enantiomer at that bond; or a pharmaceutically acceptable salt or hydrate thereof.

In some aspects, ring A is a piperazinyl ring. In some aspects, ring A is a diazepanyl ring.

In some aspects of Formula IA, Riis:

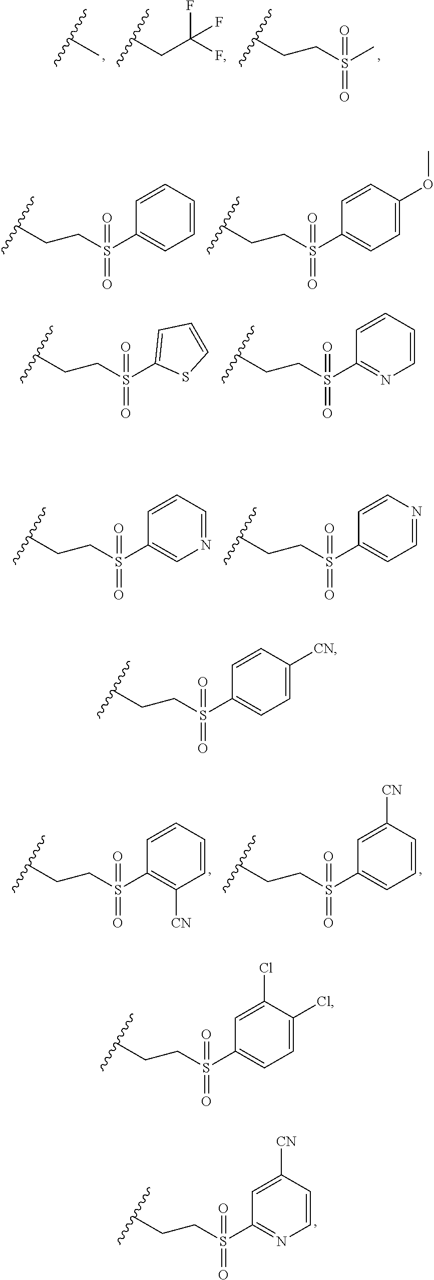

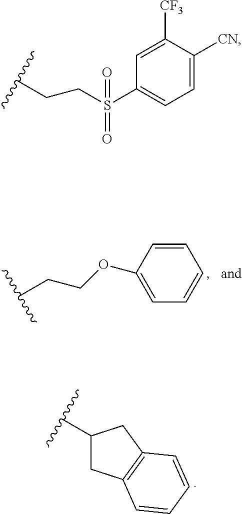 [SMILES: *CCS(=O)(=O)c1ccc(C#N)c(C(F)(F)F)c1 ; *CCOc1ccccc1 ; "," ; "and" ; *C1Cc2ccccc2C1 ; "."]

In some aspects of Formula IA, R₂ is:

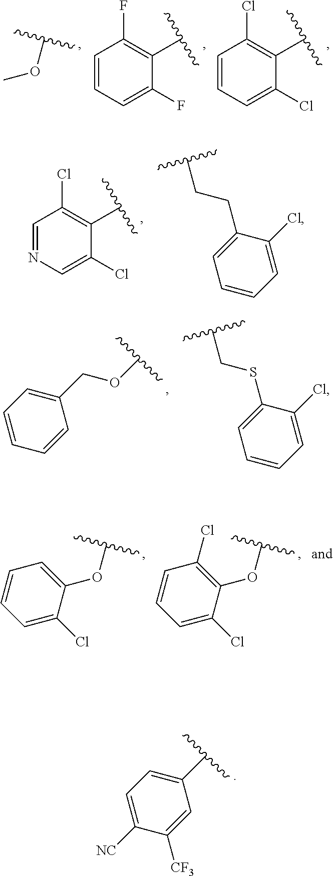

In some aspects, a compound of Formula IB is provided:

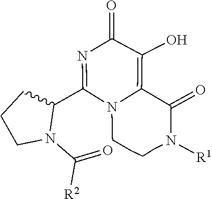

wherein: R¹ is C1-C3 alkyl, C1-C3 alkoxy, C1-C3 haloalkyl, C1-C3 alkylsulfonyl, or 9- to 10-membered cycloaryl, wherein R¹ can further be independently substituted with one or more R^{x} groups; R^{x} is sulfonyl, oxo, C1-C2 haloalkyl, 5- to 6-membered aryl, or 5- to 6-membered heteroaryl, wherein R^{x} can independently further be substituted with one or more R^{a} groups; R^{a} is C1-C2 alkyl, 5- to 6-membered aryl, 5- to 6-membered heteroaryl, C1-C2 haloalkyl, wherein R^{a} can independently be substituted with one or more Rai groups; Rai is halo, C1-C2 alkoxy, cyano, or C1-C2 haloalkyl; R² is C1-C2 alkyl, 5- to 6-membered aryl, oxo, 5- to 6-membered heteroaryl, C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further substituted with one or more R^{b} groups; R^{b} is halo or 5- to 6-membered aryl; R^{b1} is halo; and wherein the wavy line indicates either R or S enantiomer at that bond; or a pharmaceutically acceptable salt or hydrate thereof.

In some aspects of Formula B₁, R₁ is:

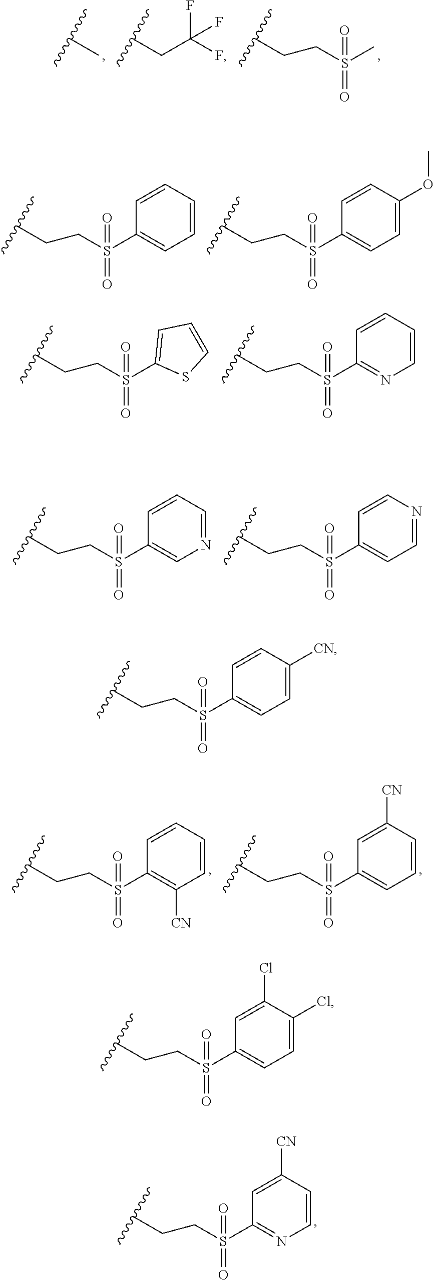

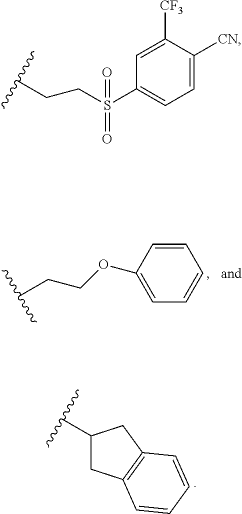 [SMILES: *CCS(=O)(=O)c1ccc(C#N)c(C(F)(F)F)c1 ; *CCOc1ccccc1 ; "," ; "and" ; *C1Cc2ccccc2C1 ; "."]

In some aspects of Formula B1, R₂ is

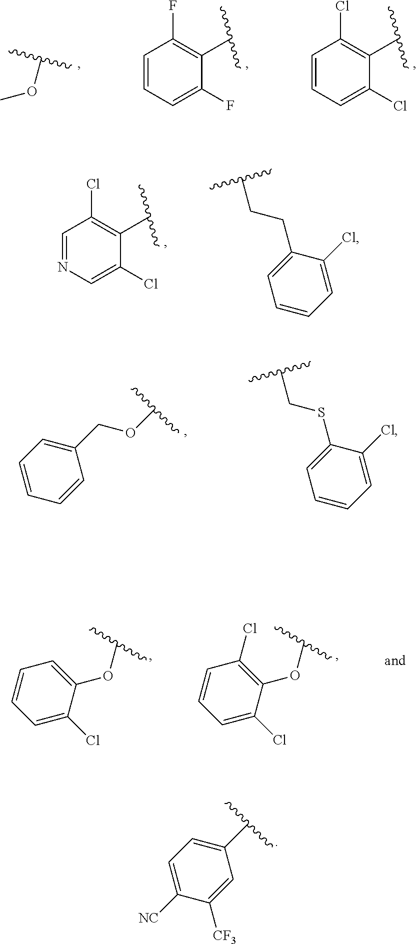

In some aspects, a compound of Formula IC is provided:

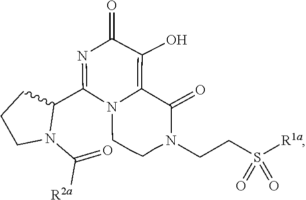

wherein: R^{1a} is C1-C3 alkyl, 5- to 6-membered aryl, or 5- to 6-membered heteroaryl, wherein R¹ can be further independently substituted with one or more R^{x} groups; R^{x} is cyano, halo, or C1-C2 alkoxy; R^{2a} is C1-C2 alkyl, 5- to 6-membered aryl, oxo, 5- to 6-membered heteroaryl, or C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further substituted with one or more R^{b} groups; R^{b} is halo or 5- to 6-membered aryl; R^{b1} is halo; and wherein the squiggly/wavy line indicates either R or S enantiomer at that bond; or a pharmaceutically acceptable salt or hydrate thereof.

In some aspects of Formula IC, R₁ₐ is:

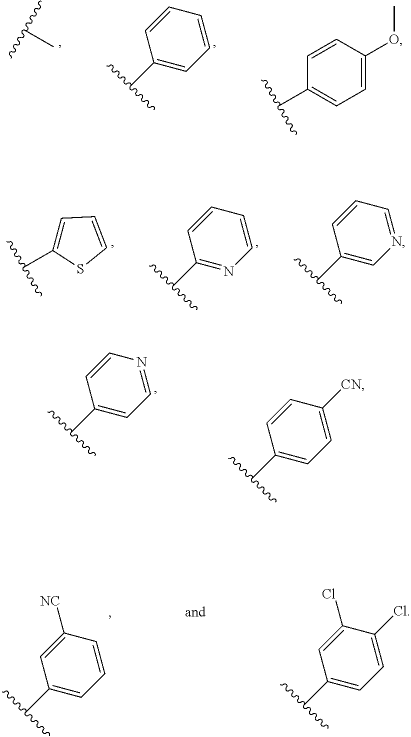

In some aspects of Formula IC, wherein R₂ₐ is:

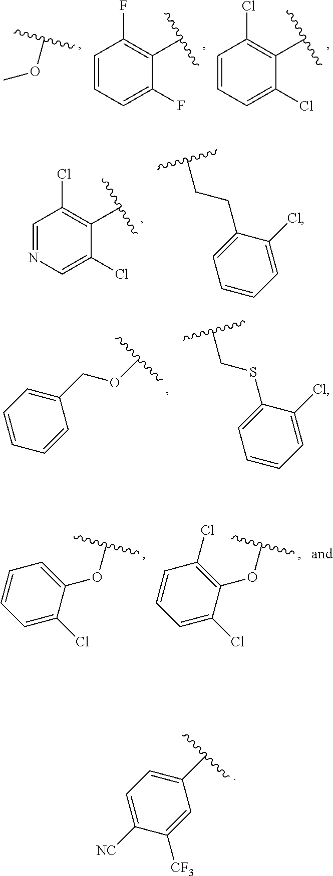

In some aspects, the compound has a structure represented by a formula:

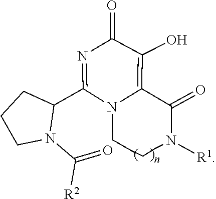

In some aspects, the compound has a structure represented by a formula:

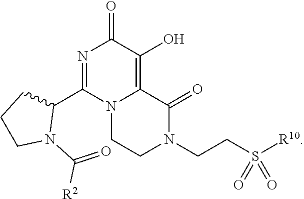

In a further aspect, R¹⁰ is a structure selected from:

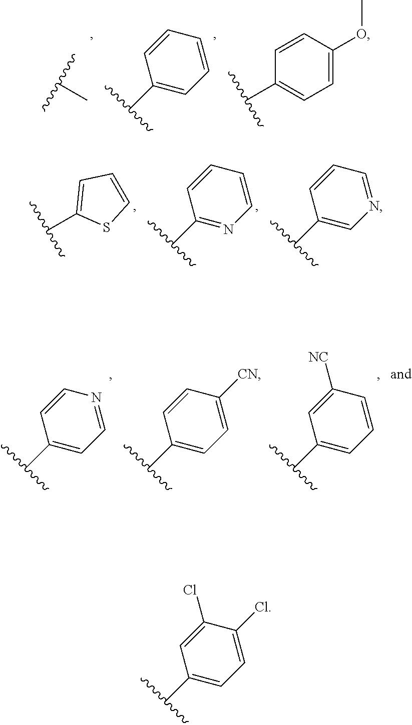

In a further aspect, R² is a structure selected from:

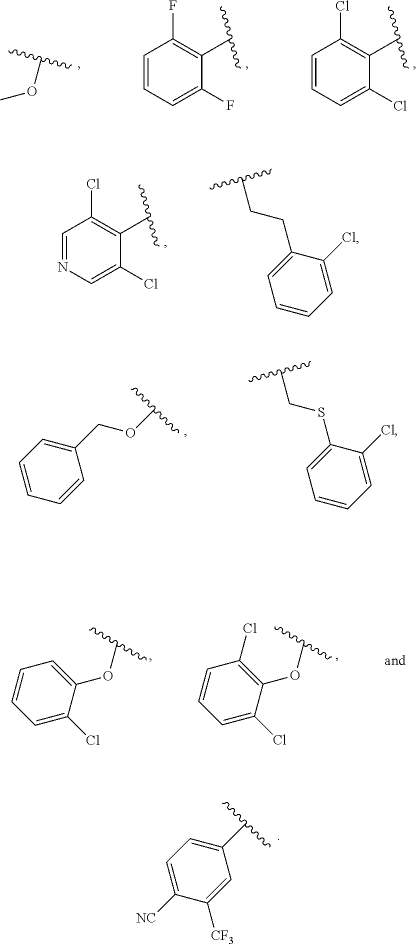

In some aspects, the compound has a structure:

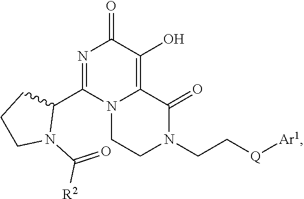

wherein: Q is O or SO₂.

In some aspects, the compound has a structure:

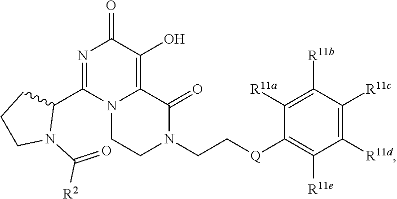

wherein: each of R^{11a}, R^{11b}, R^{11c}, R^{11d}, and R^{11e} is independently selected from hydrogen, halogen, —CN, C1-C2 alkyl, C1-C2 haloalkyl, and —C1-C2 alkoxy, provided that at least two of R^{11a}, R^{11b}, R^{11c}, R^{11d}, and R^{11e} are hydrogen.

In some aspects, the compound of the present disclosure is selected from the group consisting of:

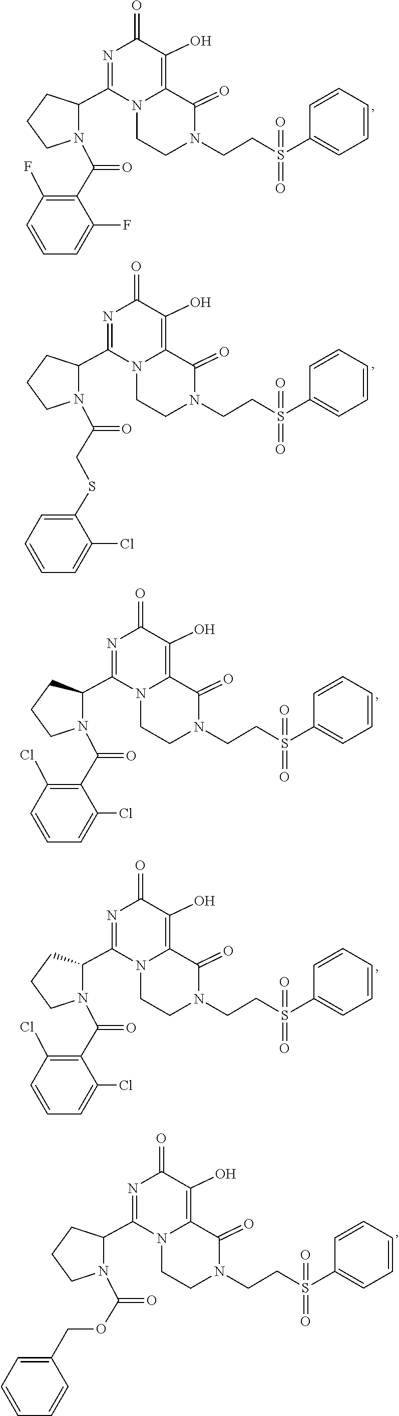

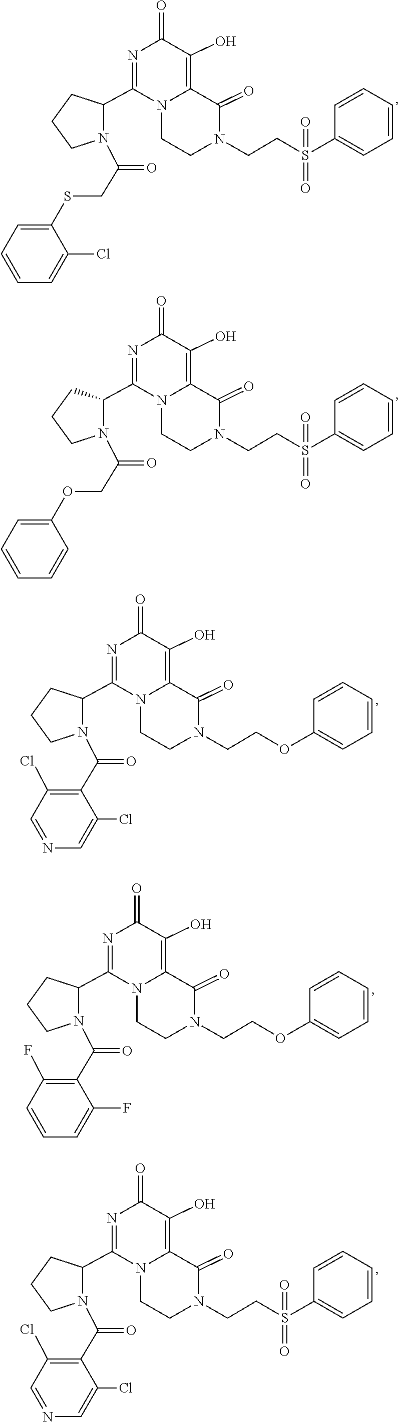 [SMILES: O=C1c2c(O)c(=O)nc(C3CCCN3C(=O)CSc3ccccc3Cl)n2CCN1CCS(=O)(=O)c1ccccc1 ; O=C1c2c(O)c(=O)nc([C@H]3CCCN3C(=O)COc3ccccc3)n2CCN1CCS(=O)(=O)c1ccccc1 ; O=C1c2c(O)c(=O)nc(C3CCCN3C(=O)c3c(Cl)cncc3Cl)n2CCN1CCOc1ccccc1 ; O=C1c2c(O)c(=O)nc(C3CCCN3C(=O)c3c(F)cccc3F)n2CCN1CCOc1ccccc1 ; O=C1c2c(O)c(=O)nc(C3CCCN3C(=O)c3c(Cl)cncc3Cl)n2CCN1CCS(=O)(=O)c1ccccc1]

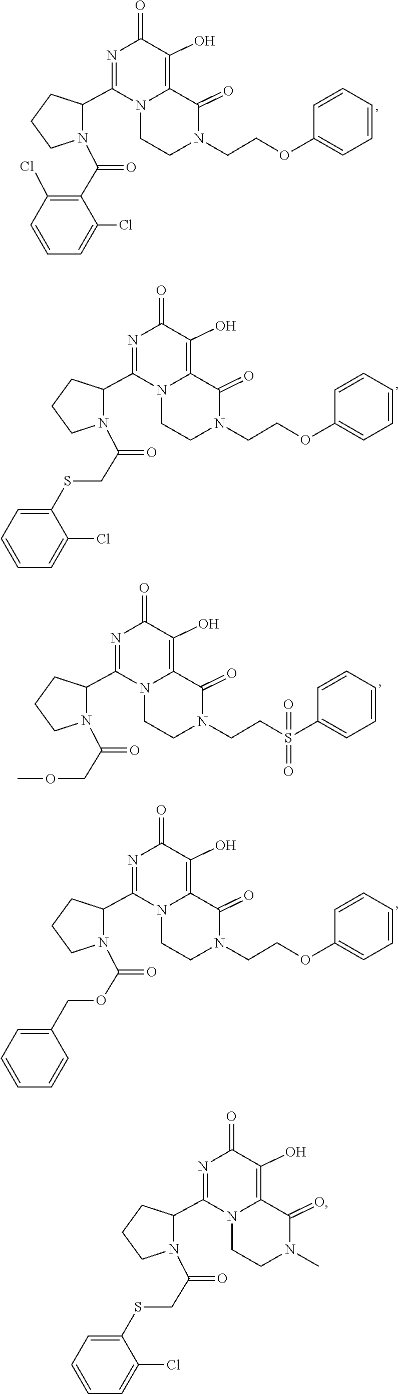 [SMILES: O=C1c2c(O)c(=O)nc(C3CCCN3C(=O)c3c(Cl)cccc3Cl)n2CCN1CCOc1ccccc1 ; O=C1c2c(O)c(=O)nc(C3CCCN3C(=O)CSc3ccccc3Cl)n2CCN1CCOc1ccccc1 ; COCC(=O)N1CCCC1c1nc(=O)c(O)c2n1CCN(CCS(=O)(=O)c1ccccc1)C2=O ; O=C1c2c(O)c(=O)nc(C3CCCN3C(=O)OCc3ccccc3)n2CCN1CCOc1ccccc1 ; CN1CCn2c(C3CCCN3C(=O)CSc3ccccc3Cl)nc(=O)c(O)c2C1=O]

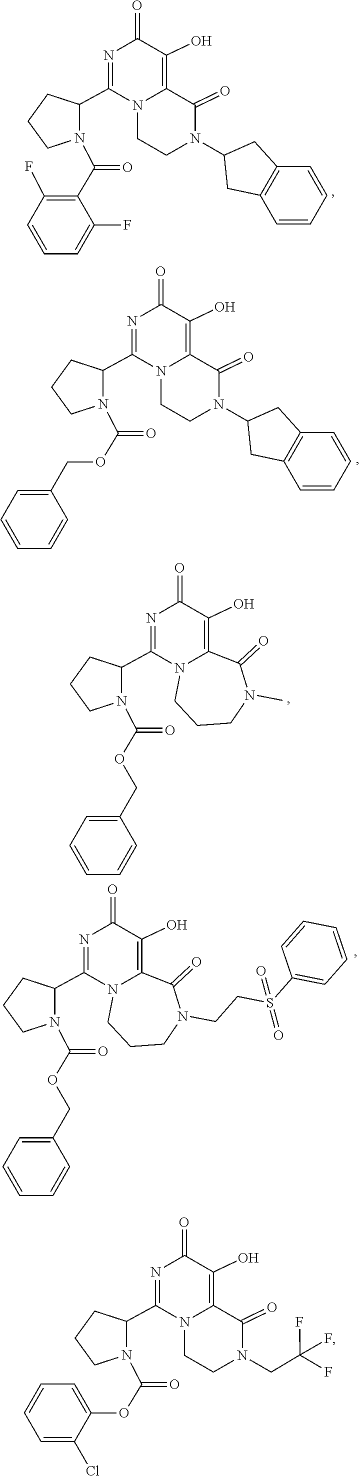

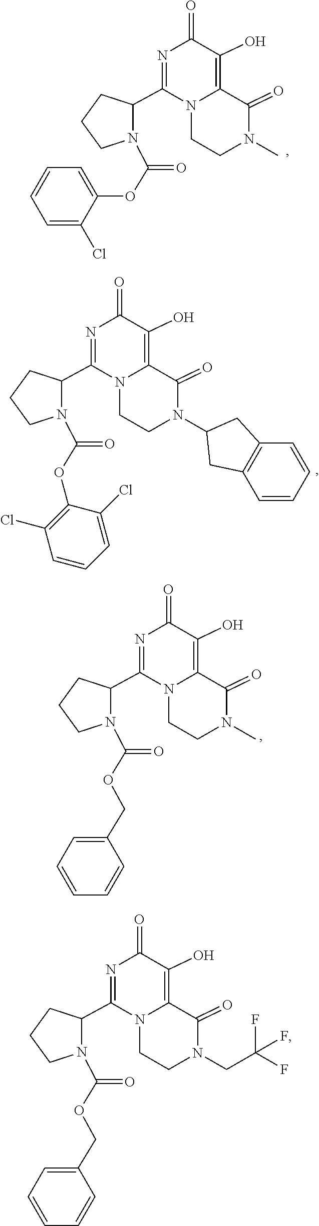

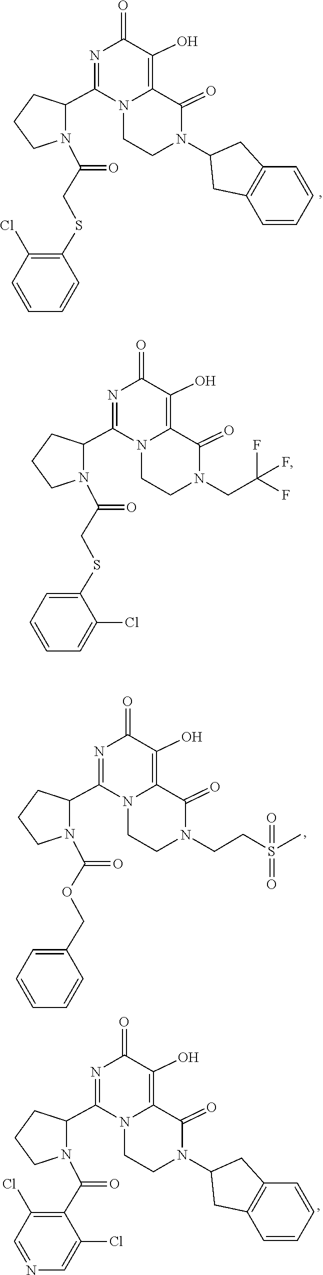 [SMILES: O=C1c2c(O)c(=O)nc(C3CCCN3C(=O)CSc3ccccc3Cl)n2CCN1C1Cc2ccccc2C1 ; O=C1c2c(O)c(=O)nc(C3CCCN3C(=O)CSc3ccccc3Cl)n2CCN1CC(F)(F)F ; CS(=O)(=O)CCN1CCn2c(C3CCCN3C(=O)OCc3ccccc3)nc(=O)c(O)c2C1=O ; O=C1c2c(O)c(=O)nc(C3CCCN3C(=O)c3c(Cl)cncc3Cl)n2CCN1C1Cc2ccccc2C1]

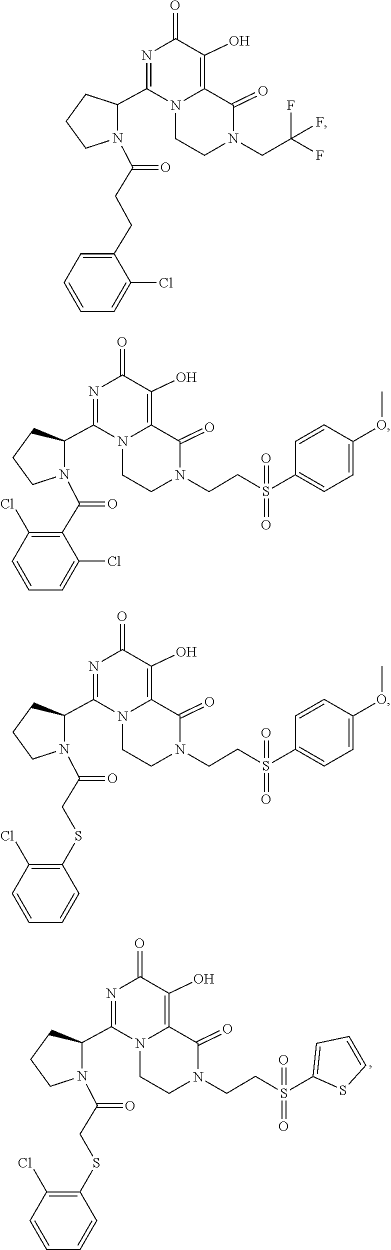

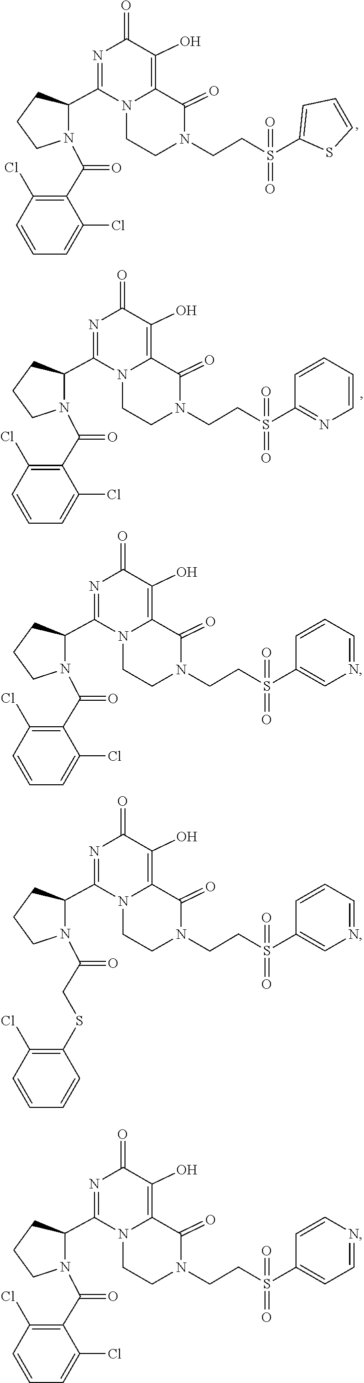

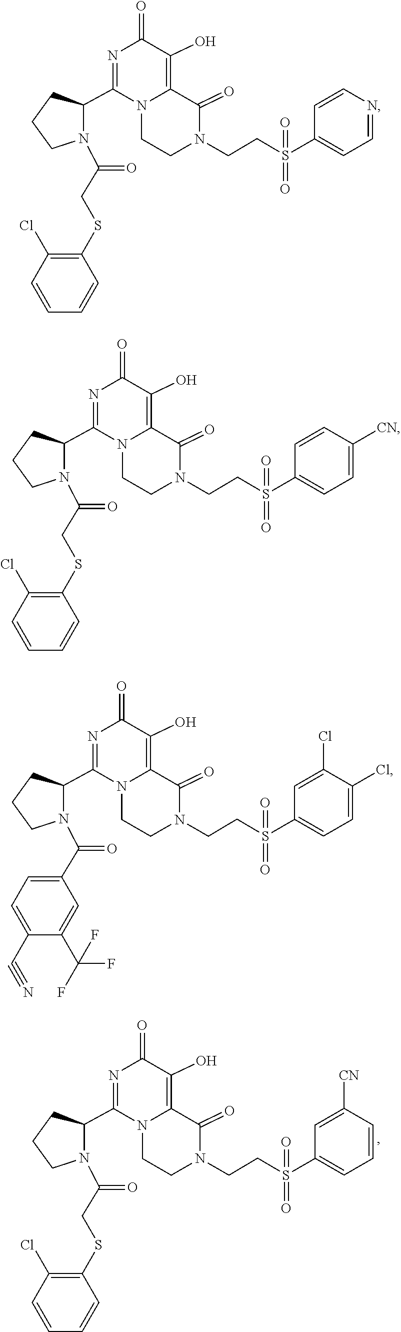 [SMILES: O=C1c2c(O)c(=O)nc([C@@H]3CCCN3C(=O)CSc3ccccc3Cl)n2CCN1CCS(=O)(=O)c1ccncc1 ; N#Cc1ccc(S(=O)(=O)CCN2CCn3c([C@@H]4CCCN4C(=O)CSc4ccccc4Cl)nc(=O)c(O)c3C2=O)cc1 ; N#Cc1ccc(C(=O)N2CCC[C@H]2c2nc(=O)c(O)c3n2CCN(CCS(=O)(=O)c2ccc(Cl)c(Cl)c2)C3=O)cc1C(F)(F)F ; N#Cc1cccc(S(=O)(=O)CCN2CCn3c([C@@H]4CCCN4C(=O)CSc4ccccc4Cl)nc(=O)c(O)c3C2=O)c1]

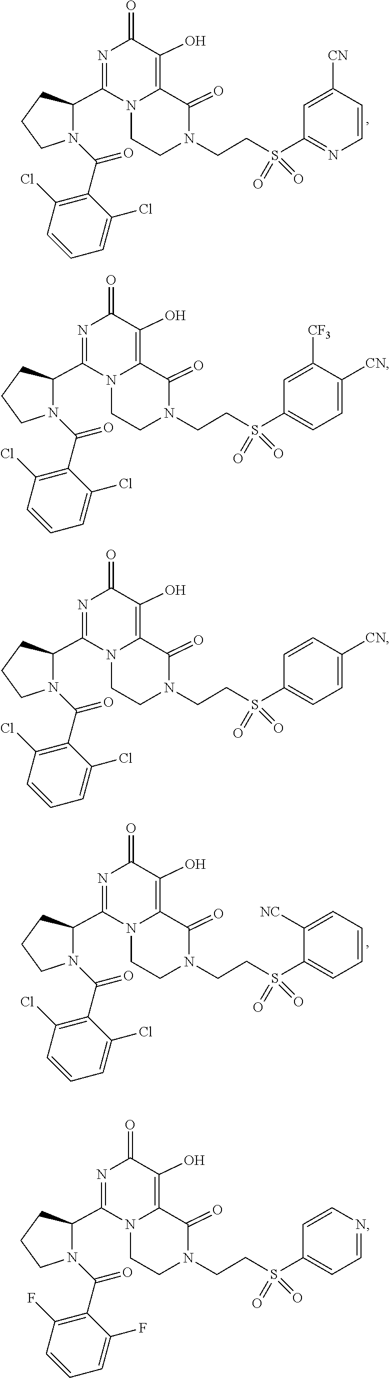

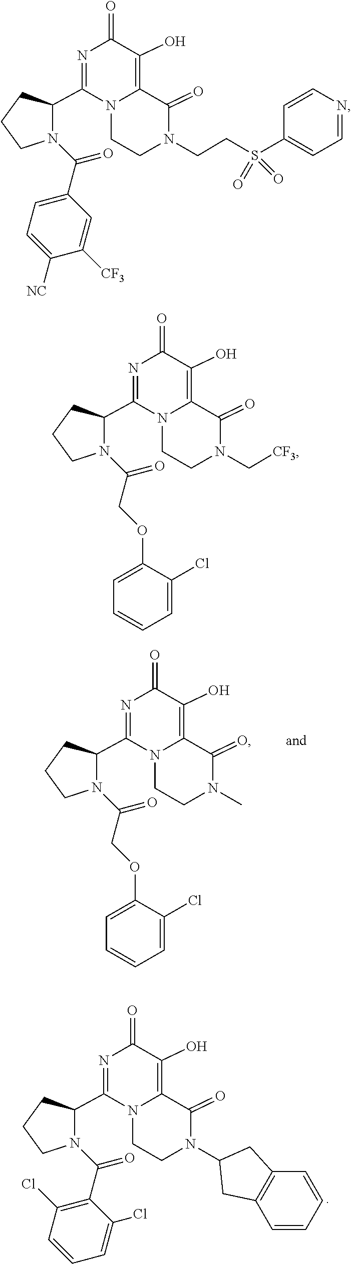

In some aspects, the compound of the present disclosure is selected from the group consisting of:

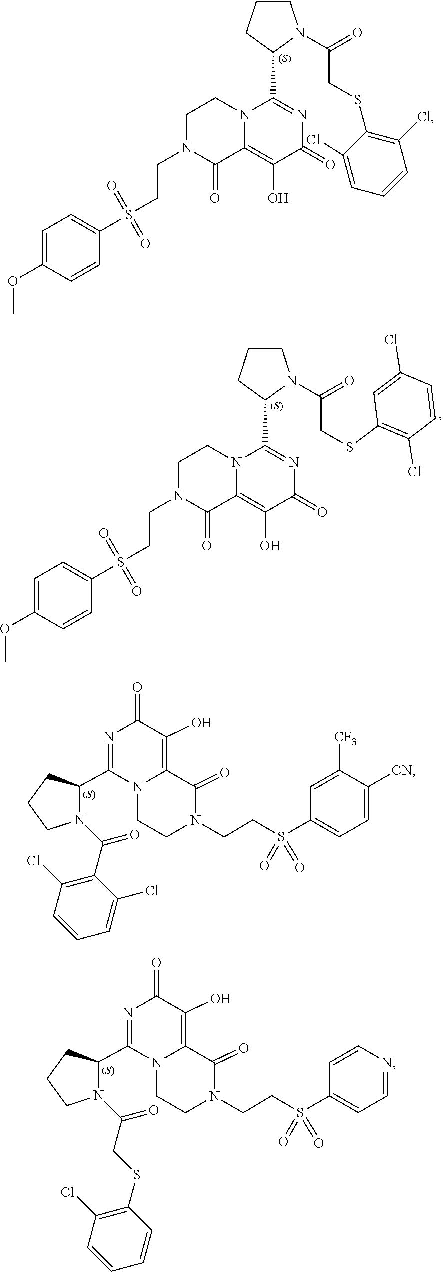

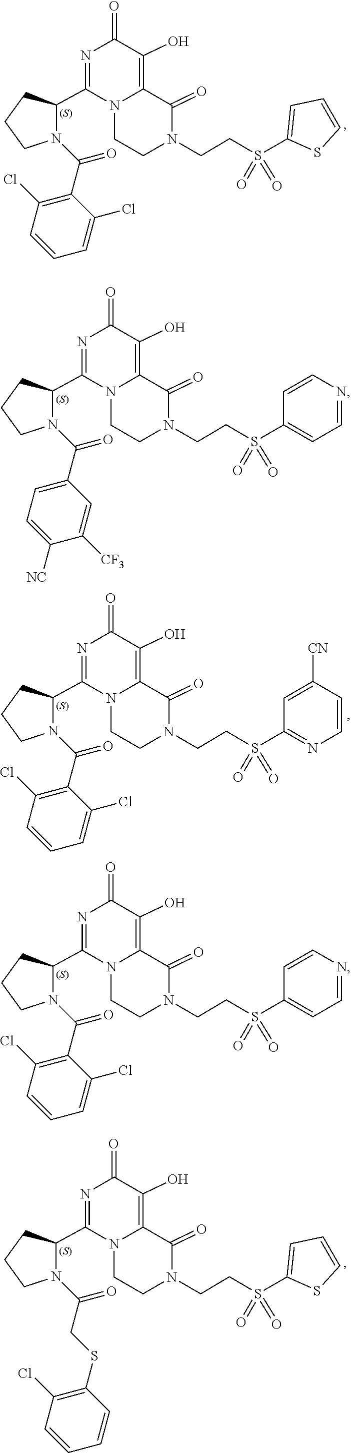

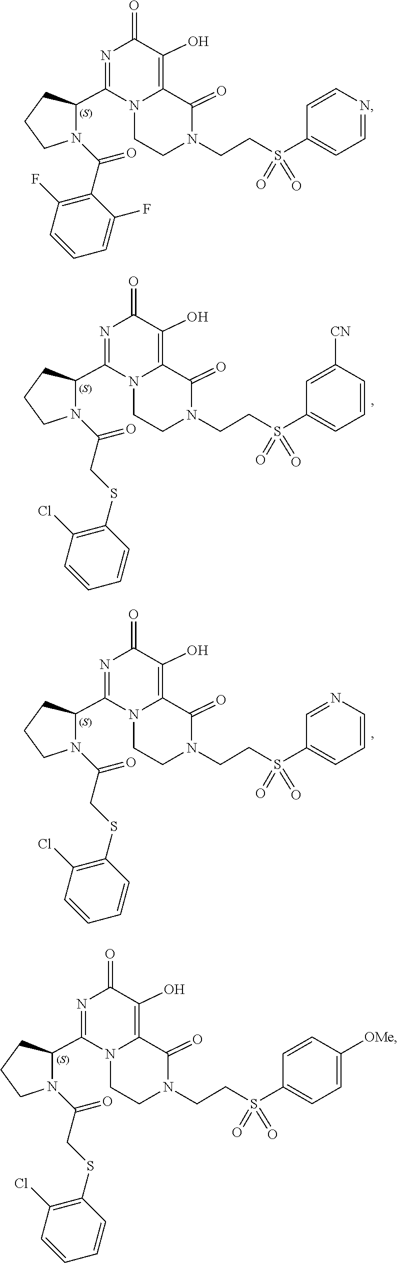

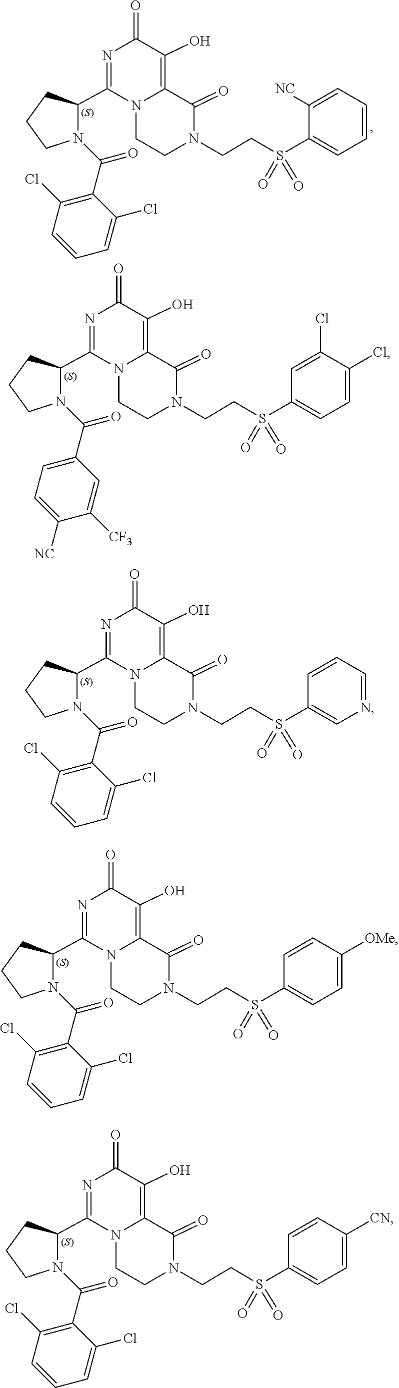

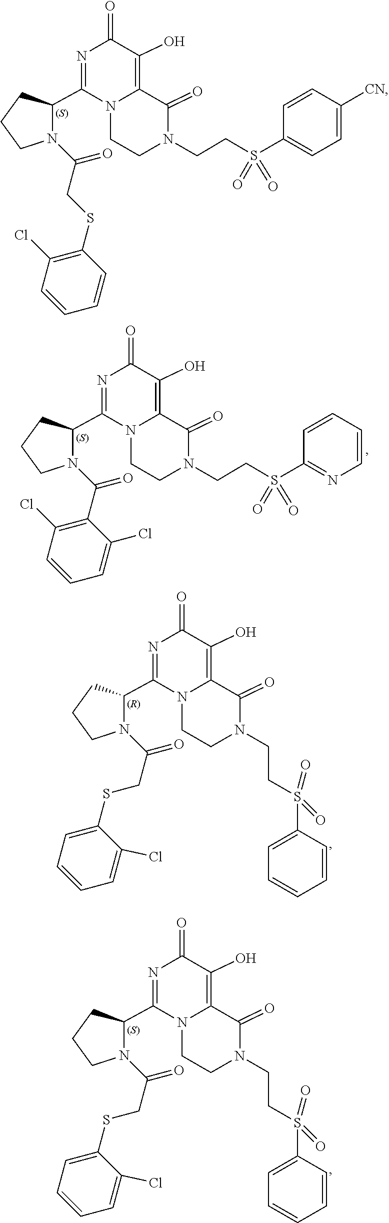

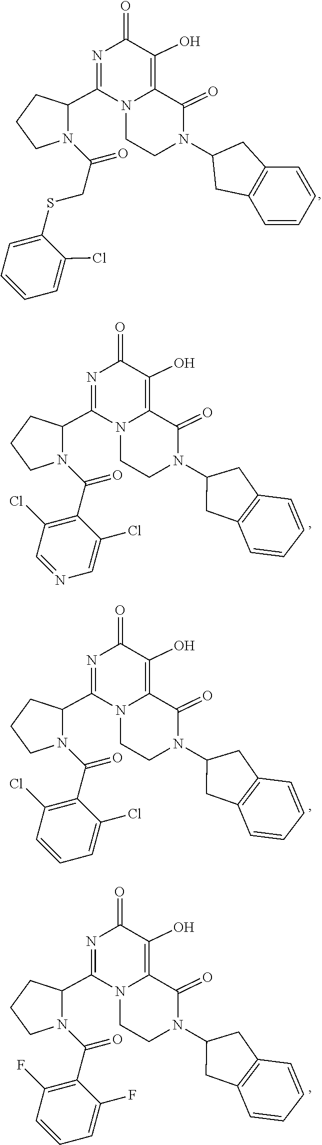

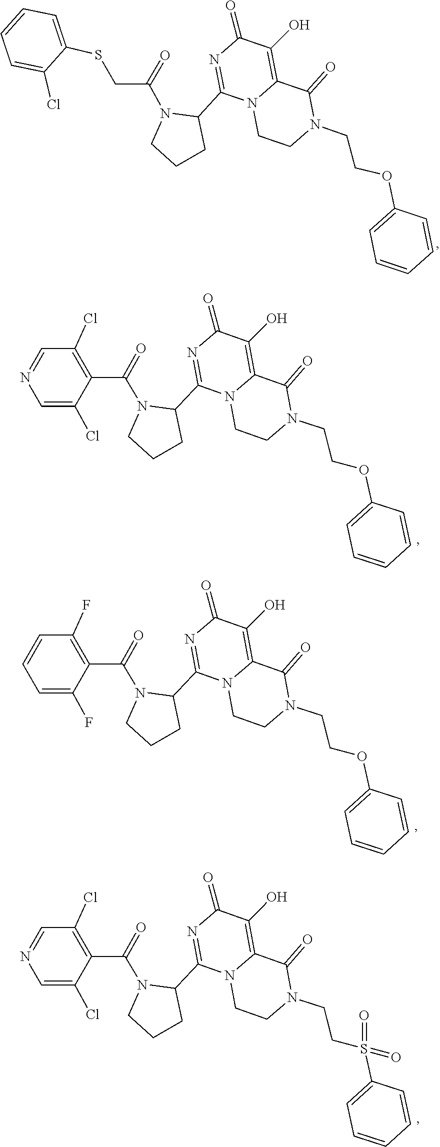 [SMILES: O=C1c2c(O)c(=O)nc(C3CCCN3C(=O)CSc3ccccc3Cl)n2CCN1CCOc1ccccc1 ; O=C1c2c(O)c(=O)nc(C3CCCN3C(=O)c3c(Cl)cncc3Cl)n2CCN1CCOc1ccccc1 ; O=C1c2c(O)c(=O)nc(C3CCCN3C(=O)c3c(F)cccc3F)n2CCN1CCOc1ccccc1 ; O=C1c2c(O)c(=O)nc(C3CCCN3C(=O)c3c(Cl)cncc3Cl)n2CCN1CCS(=O)(=O)c1ccccc1]

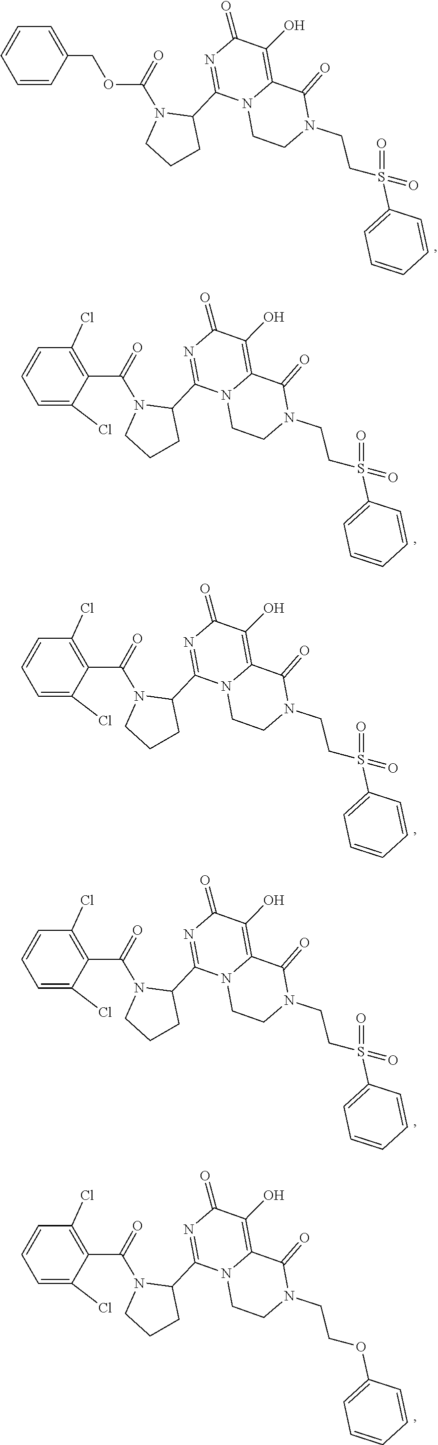 [SMILES: O=C1c2c(O)c(=O)nc(C3CCCN3C(=O)OCc3ccccc3)n2CCN1CCS(=O)(=O)c1ccccc1 ; O=C1c2c(O)c(=O)nc(C3CCCN3C(=O)c3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCOc1ccccc1]

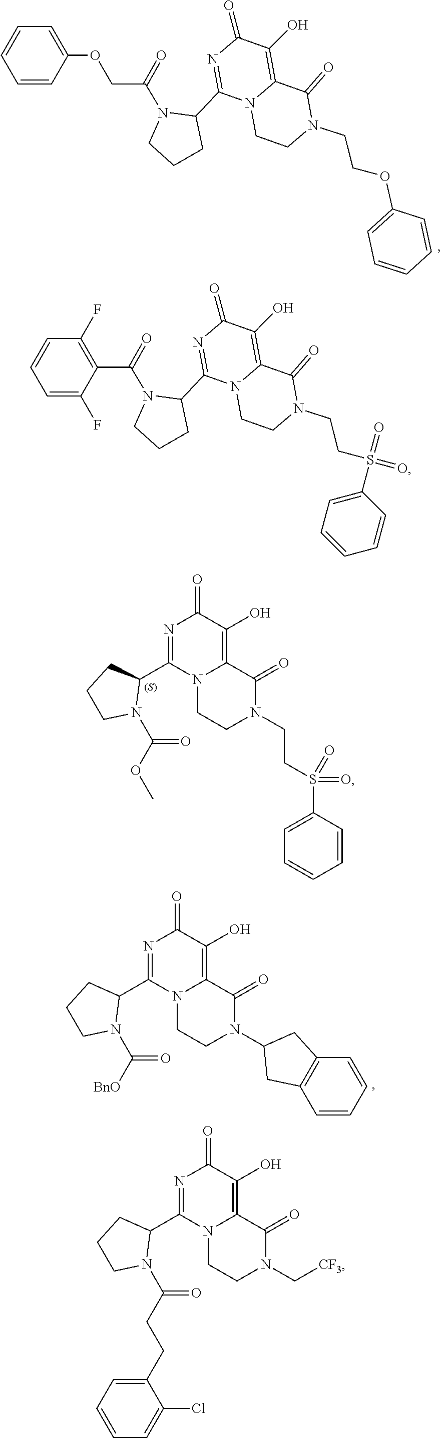

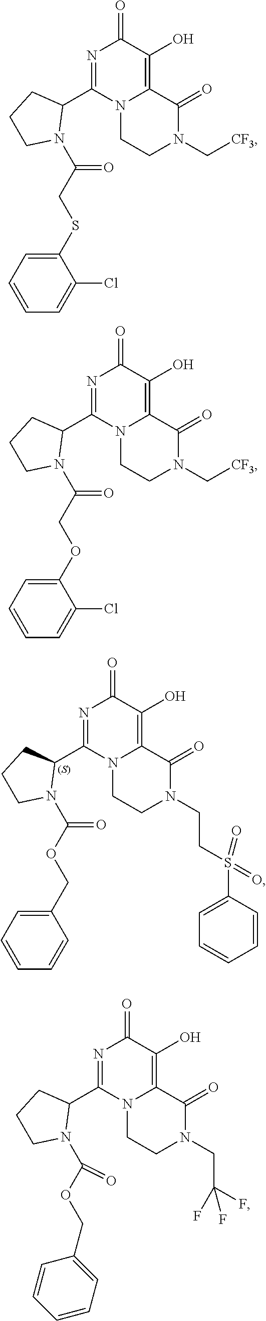

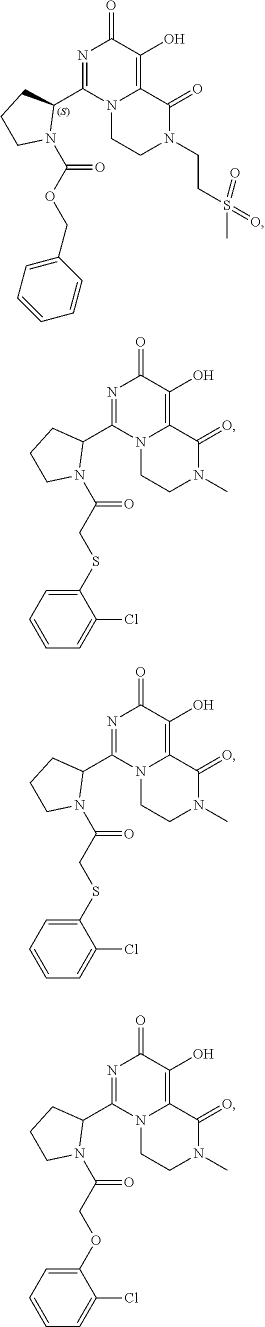

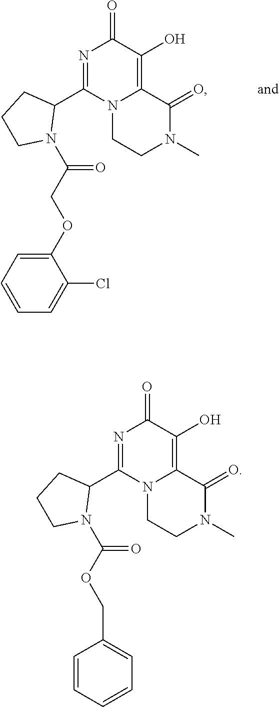 [SMILES: CN1CCn2c(C3CCCN3C(=O)COc3ccccc3Cl)nc(=O)c(O)c2C1=O ; "and" ; CN1CCn2c(C3CCCN3C(=O)OCc3ccccc3)nc(=O)c(O)c2C1=O]

In one aspect, the wavy line indicates either R or S enantiomer at that bond. In a further aspect, the wavy line indicates R enantiomer at that bond. In a still further aspect, the wavy line indicates S enantiomer at that bond.

In one aspect, n is 1 or 2. In a further aspect, n is 1. In a still further aspect, n is 2.

### a. A Groups

In one aspect, A is a 6-7 membered heterocycle. Thus, in a further aspect, A is a 6-membered heterocycle. In a still further aspect, A is a 7-membered heterocycle.

In a further aspect, A is a piperazinyl ring or a diazepanyl ring. In a still further aspect, A is a piperazinyl ring. In yet a further aspect, A is a diazepanyl ring.

### b. Q Groups

In one aspect, Q is 0 or SO₂. In a further aspect, Q is O. In a still further aspect, Q is SO₂.

### c. R^{x} Groups

In one aspect, R^{x} is sulfonyl, oxo, C1-C2 haloalkyl, 5- to 6-membered aryl, or 5- to 6-membered heteroaryl, wherein R^{x} can independently further be substituted with one or more R^{a} groups. In a further aspect, R^{x} is sulfonyl, oxo, —CCl₃, —CHCl₂, —CH₂Cl, —CH₂CH₂Cl, —CF₃, —CHF₂, —CH₂F, —CH₂CH₂F, 5- to 6-membered aryl, or 5- to 6-membered heteroaryl, wherein R^{x} can independently further be substituted with one or more R^{a} groups. In a still further aspect, R^{x} is sulfonyl, oxo, —CCl₃, —CHCl₂, —CH₂Cl, —CF₃, —CHF₂, —CH₂F, 5- to 6-membered aryl, or 5- to 6-membered heteroaryl, wherein R^{x} can independently further be substituted with one or more R^{a} groups.

### d. R^{Y} Groups

In one aspect, R^{y} is halo, oxo, C1-C2 alkyl, sulfidyl, cyano, C1-C2 haloalkyl, or 5- to 6-membered aryl, wherein R^{y} can independently further be substituted with one or more R^{b} groups. In a further aspect, R^{y} is —Cl, —Br, —F, oxo, methyl, ethyl, sulfidyl, —CN, —CCl₃, —CHCl₂, —CH₂Cl, —CH₂CH₂Cl, —CF₃, —CHF₂, —CH₂F, —CH₂CH₂F, or 5- to 6-membered aryl, wherein R^{y} can independently further be substituted with one or more R^{b} groups. In a still further aspect, R^{y} is —Cl, —Br, —F, oxo, methyl, sulfidyl, —CN, —CCl₃, —CHCl₂, —CH₂Cl, —CF₃, —CHF₂, —CH₂F, or 5- to 6-membered aryl, wherein R^{y} can independently further be substituted with one or more R^{b} groups.

### e. R^{A} Groups

In one aspect, R^{a} is C1-C2 alkyl, 5- to 6-membered aryl, 5- to 6-membered heteroaryl, or C1-C2 haloalkyl, wherein R^{a} can independently be substituted with one or more R^{a1} groups. In a further aspect, R^{a} is methyl, ethyl, 5- to 6-membered aryl, 5- to 6-membered heteroaryl, —CCl₃, —CHCl₂, —CH₂Cl, —CH₂CH₂Cl, —CF₃, —CHF₂, —CH₂F, or —CH₂CH₂F, wherein R^{a} can independently be substituted with one or more R^{a1} groups. In a still further aspect, R^{a} is methyl, 5- to 6-membered aryl, 5- to 6-membered heteroaryl, —CCl₃, —CHCl₂, —CH₂Cl, —CF₃, —CHF₂, or —CH₂F, wherein R^{a} can independently be substituted with one or more R^{a1} groups.

### f. R^{a1} Groups

In one aspect, R^{a1} is halo, C1-C2 alkoxy, cyano, or C1-C2 haloalkyl. In a further aspect, R^{a1} is —Cl, —Br, —F, —OCH₃, —OCH₂CH₃, —CN, —CCl₃, —CHCl₂, —CH₂Cl, —CH₂CH₂Cl, —CF₃, —CHF₂, —CH₂F, or —CH₂CH₂F. In a still further aspect, R^{a1} is-Cl, —Br, —F, —OCH₃, —CN, —CCl₃, —CHCl₂, —CH₂Cl, —CF₃, —CHF₂, or —CH₂F.

### g. R^{B} Groups

In one aspect, R^{b} is halo or 5- to 6-membered aryl. In a further aspect, R^{b} is —Cl, —Br, —F, or 5- to 6-membered aryl. In a still further aspect, R^{b} is —Cl, —F, or 5- to 6-membered aryl. In yet a further aspect, R^{b} is —Cl or 5- to 6-membered aryl. In an even further aspect, R^{b} is —F or 5- to 6-membered aryl.

In various aspect, R^{b} is halo. In a further aspect, R^{b} is —Cl, —Br, or —F. In a still further aspect, R^{b} is —Cl or —F. In yet a further aspect, R^{b} is —C1. In an even further aspect, R^{b} is —F.

In various aspects, R^{b} is 5- or 6-membered aryl. In a further aspect, R^{b} is a 5-membered aryl. In a still further aspect, R^{b} is a 6-membered aryl.

### h. R^{B}1 Groups

In one aspect, R^{b1} is halo. In a further aspect, R^{b1} is —Cl, —Br, or —F. In a still further aspect, R^{b1} is —Cl or —F. In yet a further aspect, R^{b1} is —C1. In an even further aspect, R^{b1} is —F.

### i. R¹ Groups

In one aspect, R¹ is C1-C3 alkyl, C1-C3 alkoxy, C1-C3 haloalkyl, C1-C3 alkylsulfonyl, or a 9- to 10-membered cycloaryl, wherein R¹ can further be independently substituted with one or more R^{x} groups.

In one aspect, R¹ is C1-C3 alkyl, C1-C3 haloalkyl, —(C1-C3 alkyl)OR¹⁰, —(C1-C3 alkyl)SO₂R¹⁰, or Cy¹. In a further aspect, R¹ is methyl, ethyl, n-propyl, isopropyl, —CCl₃, —CHCl₂, —CH₂Cl, —CH₂CH₂Cl, —CH₂CH₂CH₂Cl, —(CH)(CH₃)CH₂Cl, —CF₃, —CHF₂, —CH₂F, —CH₂CH₂F, —CH₂CH₂CH₂F, —(CH)(CH₃)CH₂F, —CH₂OR¹⁰, —CH₂CH₂OR¹⁰, —CH₂CH₂CH₂OR¹⁰, —(CH)(CH₃)CH₂OR¹⁰, —CH₂SO₂R¹⁰, —CH₂CH₂SO₂R¹⁰, —CH₂CH₂CH₂SO₂R¹⁰, —(CH)(CH₃)CH₂SO₂R¹⁰, or Cy¹. In a still further aspect, R¹ is methyl, ethyl, —CCl₃, —CHCl₂, —CH₂Cl, —CH₂CH₂Cl, —CF₃, —CHF₂, —CH₂F, —CH₂CH₂F, —CH₂OR¹⁰, —CH₂CH₂OR¹⁰, —CH₂SO₂R¹⁰, —CH₂CH₂SO₂R¹⁰, or Cy¹. In yet a further aspect, R¹ is methyl, —CCl₃, —CHCl₂, —CH₂Cl, —CF₃, —CHF₂, —CH₂F, —CH₂OR¹⁰, —CH₂SO₂R¹⁰, or Cy¹.

In various aspects, R¹ is a structure selected from:

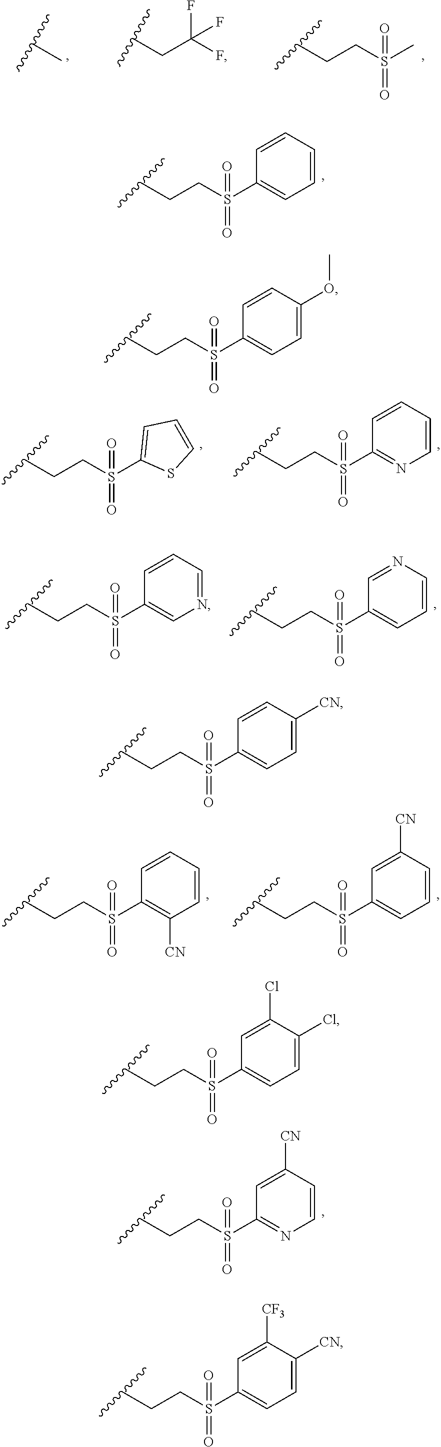

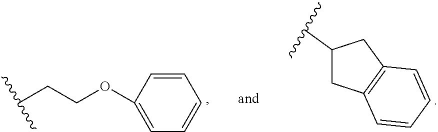 [SMILES: *CCOc1ccccc1 ; *C1Cc2ccccc2C1]

### j. R² Groups

In one aspect, R² is C1-C2 alkyl, 5- to 6-membered aryl, oxo, 5- to 6-membered heteroaryl, or C1-C2 alkoxy, wherein R² can further be independently substituted with one or more R^{y} groups.

In one aspect, R² is C1-C2 alkyl, —(C1-C2 alkyl)Ar², —O(C1-C2 alkyl), —O(C1-C2 alkyl)Ar², —(C1-C2 alkyl)OAr², —S(C1-C2 alkyl), —S(C1-C2 alkyl)Ar², —(C1-C2 alkyl)SAr², or Ar². In a further aspect, R² is methyl, ethyl, —CH₂Ar², —CH₂CH₂Ar², —OCH₃, —OCH₂CH₃, —OCH₂Ar², —OCH₂CH₂Ar², —CH₂OAr², —CH₂CH₂OAr², —SCH₃, —SCH₂CH₃, —SCH₂Ar², —SCH₂CH₂Ar², —CH₂SAr², —CH₂CH₂SAr², or Ar². In a still further aspect, R² is methyl, —CH₂Ar², —OCH₃, —OCH₂Ar², —CH₂OAr², —SCH₃, —SCH₂Ar², —CH₂SAr², or Ar².

In various aspects, R² is a structure selected from:

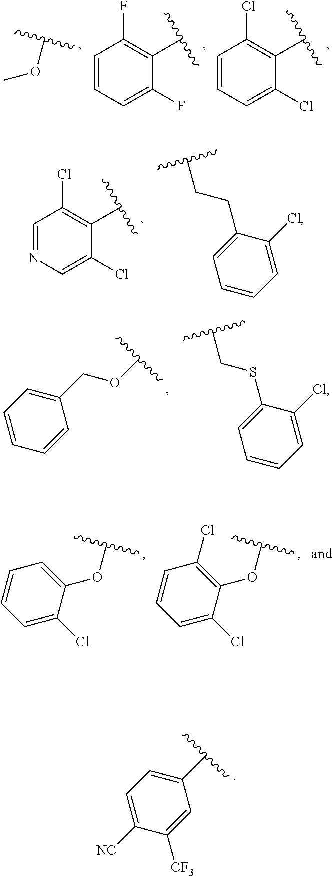

### k. R¹⁰ Groups

In one aspect, R¹⁰ is C1-C2 alkyl or Ar¹. In a further aspect, R¹⁰ is methyl, ethyl, or Ar¹. In a still further aspect, R¹⁰ is ethyl or Ar¹. In yet a further aspect, R¹⁰ is methyl or Ar¹.

In a further aspect, R¹⁰ is C1-C2 alkyl. In a still further aspect, R¹⁰ is ethyl. In a still further aspect, R¹⁰ is methyl.

In a further aspect, R¹⁰ is Ar¹.

In various aspects, R¹⁰ is a structure selected from:

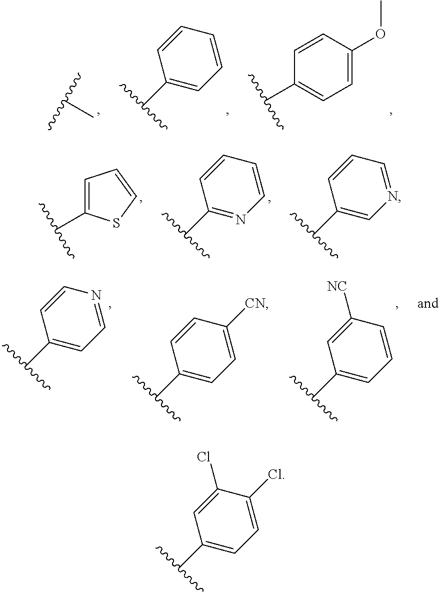

### 1. R^{11A}, R^{11B}, R^{11C}, R^{11D}, and R^{11E} GROUPS

In one aspect, each of R^{11a}, R^{11b}, R^{11c}, R^{11d}, and R^{11e} is independently selected from hydrogen, halogen, —CN, C1-C2 alkyl, C1-C2 haloalkyl, and —C1-C2 alkoxy, provided that at least two of R^{11a}, R^{11b}, R^{11c}, R^{11d}, and R^{11e} are hydrogen. In a further aspect, each of R^{11a}, R^{11b}, R^{11c}, R^{11d}, and R^{11e} is independently selected from hydrogen, —F, —Cl, —Br, —CN, methyl, ethyl, —CCl₃, —CHCl₂, —CH₂Cl, —CH₂CH₂Cl, —CF₃, —CHF₂, —CH₂F, —CH₂CH₂F, —OCH₃, and —OCH₂CH₃. In a still further aspect, each of R^{11a}, R^{11b}, R^{11c}, R^{11d}, and R^{11e} is independently selected from hydrogen, —F, —Cl, —Br, —CN, methyl, —CCl₃, —CHCl₂, —CH₂Cl, —CF₃, —CHF₂, —CH₂F, and —OCH₃.

In a further aspect, at least two of R^{11a}, R^{11b}, R^{11c}, R^{11d}, and R^{11e} are hydrogen. In a still further aspect, at least three of R^{11a}, R^{11b}, R^{11c}, R^{11d}, and R^{11e} are hydrogen. In yet a further aspect, at least four of R^{11a}, R^{11b}, R^{11c}, R^{11d}, and R^{11e} are hydrogen.

In a further aspect, one of R^{11a}, R^{11b}, R^{11c}, R^{11d}, and R^{11e} is hydrogen. In a still further aspect, two of R^{11a}, R^{11b}, R^{11c}, R^{11d}, and R^{11e} are hydrogen. In yet a further aspect, three of R^{11a}, R^{11b}, R^{11c}, R^{11d}, and R^{11e} are hydrogen.

### m. AR¹ Groups

In one aspect, Ar¹ is a 5- to 6-membered aryl or a 5- to 6-membered heteroaryl, and is substituted with 0, 1, 2, or 3 groups independently selected from halogen, —CN, C1-C2 alkyl, C1-C2 haloalkyl, and —C1-C2 alkoxy. In a further aspect, Ar¹ is a 5- to 6-membered aryl or a 5- to 6-membered heteroaryl, and is substituted with 0, 1, or 2 groups independently selected from halogen, —CN, C1-C2 alkyl, C1-C2 haloalkyl, and —C1-C2 alkoxy. In a still further aspect, Ar¹ is a 5- to 6-membered aryl or a 5- to 6-membered heteroaryl, and is substituted with 0 or 1 group selected from halogen, —CN, C1-C2 alkyl, C1-C2 haloalkyl, and —C1-C2 alkoxy. In yet a further aspect, Ar¹ is a 5- to 6-membered aryl or a 5- to 6-membered heteroaryl, and is monosubstituted with a group selected from halogen, —CN, C1-C2 alkyl, C1-C2 haloalkyl, and —C1-C2 alkoxy. In an even further aspect, Ar¹ is a 5- to 6-membered aryl or a 5- to 6-membered heteroaryl, and is unsubstituted.

In various aspects, Ar¹ is a 5- to 6-membered aryl substituted with 0, 1, 2, or 3 groups independently selected from halogen, —CN, C1-C2 alkyl, C1-C2 haloalkyl, and —C1-C2 alkoxy. In a further aspect, Ar¹ is a 5- to 6-membered aryl substituted with 0, 1, or 2 groups independently selected from halogen, —CN, C1-C2 alkyl, C1-C2 haloalkyl, and —C1-C2 alkoxy. In a still further aspect, Ar¹ is a 5- to 6-membered aryl substituted with 0 or 1 group selected from halogen, —CN, C1-C2 alkyl, C1-C2 haloalkyl, and —C1-C2 alkoxy. In yet a further aspect, Ar¹ is a 5- to 6-membered aryl monosubstituted with a group selected from halogen, —CN, C1-C2 alkyl, C1-C2 haloalkyl, and —C1-C2 alkoxy. In an even further aspect, Ar¹ is an unsubstituted 5- to 6-membered aryl.

In various aspects, Ar¹ is a 5-membered aryl substituted with 0, 1, 2, or 3 groups independently selected from halogen, —CN, C1-C2 alkyl, C1-C2 haloalkyl, and —C1-C2 alkoxy. In a further aspect, Ar¹ is a 5-membered aryl substituted with 0, 1, or 2 groups independently selected from halogen, —CN, C1-C2 alkyl, C1-C2 haloalkyl, and —C1-C2 alkoxy. In a still further aspect, Ar¹ is a 5-membered aryl substituted with 0 or 1 group selected from halogen, —CN, C1-C2 alkyl, C1-C2 haloalkyl, and —C1-C2 alkoxy. In yet a further aspect, Ar¹ is a 5-membered aryl monosubstituted with a group selected from halogen, —CN, C1-C2 alkyl, C1-C2 haloalkyl, and —C1-C2 alkoxy. In an even further aspect, Ar¹ is an unsubstituted 5-membered aryl.

In various aspects, Ar¹ is a 6-membered aryl substituted with 0, 1, 2, or 3 groups independently selected from halogen, —CN, C1-C2 alkyl, C1-C2 haloalkyl, and —C1-C2 alkoxy. In a further aspect, Ar¹ is a 6-membered aryl substituted with 0, 1, or 2 groups independently selected from halogen, —CN, C1-C2 alkyl, C1-C2 haloalkyl, and —C1-C2 alkoxy. In a still further aspect, Ar¹ is a 6-membered aryl substituted with 0 or 1 group selected from halogen, —CN, C1-C2 alkyl, C1-C2 haloalkyl, and —C1-C2 alkoxy. In yet a further aspect, Ar¹ is a 6-membered aryl monosubstituted with a group selected from halogen, —CN, C1-C2 alkyl, C1-C2 haloalkyl, and —C1-C2 alkoxy. In an even further aspect, Ar¹ is an unsubstituted 6-membered aryl.

In various aspects, Ar¹ is phenyl substituted with 0, 1, 2, or 3 groups independently selected from halogen, —CN, C1-C2 alkyl, C1-C2 haloalkyl, and —C1-C2 alkoxy. In a further aspect, Ar¹ is phenyl substituted with 0, 1, or 2 groups independently selected from halogen, —CN, C1-C2 alkyl, C1-C2 haloalkyl, and —C1-C2 alkoxy. In a still further aspect, Ar¹ is a 6-membered aryl substituted with 0 or 1 group selected from halogen, —CN, C1-C2 alkyl, C1-C2 haloalkyl, and —C1-C2 alkoxy. In yet a further aspect, Ar¹ is phenyl monosubstituted with a group selected from halogen, —CN, C1-C2 alkyl, C1-C2 haloalkyl, and —C1-C2 alkoxy. In an even further aspect, Ar¹ is an unsubstituted phenyl.

In various aspects, Ar¹ is a 5- to 6-membered heteroaryl substituted with 0, 1, 2, or 3 groups independently selected from halogen, —CN, C1-C2 alkyl, C1-C2 haloalkyl, and —C1-C2 alkoxy. Examples of 5- or 6-membered heteroaryls include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, and pyrazinyl. In a further aspect, Ar¹ is a 5- to 6-membered heteroaryl substituted with 0, 1, or 2 groups independently selected from halogen, —CN, C1-C2 alkyl, C1-C2 haloalkyl, and —C1-C2 alkoxy. In a still further aspect, Ar¹ is a 5- to 6-membered heteroaryl substituted with 0 or 1 group selected from halogen, —CN, C1-C2 alkyl, C1-C2 haloalkyl, and —C1-C2 alkoxy. In yet a further aspect, Ar¹ is a 5- to 6-membered heteroaryl monosubstituted with a group selected from halogen, —CN, C1-C2 alkyl, C1-C2 haloalkyl, and —C1-C2 alkoxy. In an even further aspect, Ar¹ is an unsubstituted 5- to 6-membered heteroaryl.

In various aspects, Ar¹ is a 5-membered heteroaryl substituted with 0, 1, 2, or 3 groups independently selected from halogen, —CN, C1-C2 alkyl, C1-C2 haloalkyl, and —C1-C2 alkoxy. In a further aspect, Ar¹ is a 5-membered heteroaryl substituted with 0, 1, or 2 groups independently selected from halogen, —CN, C1-C2 alkyl, C1-C2 haloalkyl, and —C1-C2 alkoxy. In a still further aspect, Ar¹ is a 5-membered heteroaryl substituted with 0 or 1 group selected from halogen, —CN, C1-C2 alkyl, C1-C2 haloalkyl, and —C1-C2 alkoxy. In yet a further aspect, Ar¹ is a 5-membered heteroaryl monosubstituted with a group selected from halogen, —CN, C1-C2 alkyl, C1-C2 haloalkyl, and —C1-C2 alkoxy. In an even further aspect, Ar¹ is an unsubstituted 5-membered heteroaryl.

In various aspects, Ar¹ is thiophenyl substituted with 0, 1, 2, or 3 groups independently selected from halogen, —CN, C1-C2 alkyl, C1-C2 haloalkyl, and —C1-C2 alkoxy. In a further aspect, Ar¹ is thiophenyl substituted with 0, 1, or 2 groups independently selected from halogen, —CN, C1-C2 alkyl, C1-C2 haloalkyl, and —C1-C2 alkoxy. In a still further aspect, Ar¹ is thiophenyl substituted with 0 or 1 group selected from halogen, —CN, C1-C2 alkyl, C1-C2 haloalkyl, and —C1-C2 alkoxy. In yet a further aspect, Ar¹ is thiophenyl monosubstituted with a group selected from halogen, —CN, C1-C2 alkyl, C1-C2 haloalkyl, and —C1-C2 alkoxy. In an even further aspect, Ar¹ is an unsubstituted thiophenyl.

In various aspects, Ar¹ is a 6-membered heteroaryl substituted with 0, 1, 2, or 3 groups independently selected from halogen, —CN, C1-C2 alkyl, C1-C2 haloalkyl, and —C1-C2 alkoxy. In a further aspect, Ar¹ is a 6-membered heteroaryl substituted with 0, 1, or 2 groups independently selected from halogen, —CN, C1-C2 alkyl, C1-C2 haloalkyl, and —C1-C2 alkoxy. In a still further aspect, Ar¹ is a 6-membered heteroaryl substituted with 0 or 1 group selected from halogen, —CN, C1-C2 alkyl, C1-C2 haloalkyl, and —C1-C2 alkoxy. In yet a further aspect, Ar¹ is a 6-membered heteroaryl monosubstituted with a group selected from halogen, —CN, C1-C2 alkyl, C1-C2 haloalkyl, and —C1-C2 alkoxy. In an even further aspect, Ar¹ is an unsubstituted 6-membered heteroaryl.

In various aspects, Ar¹ is pyridinyl substituted with 0, 1, 2, or 3 groups independently selected from halogen, —CN, C1-C2 alkyl, C1-C2 haloalkyl, and —C1-C2 alkoxy. In a further aspect, Ar¹ is pyridinyl substituted with 0, 1, or 2 groups independently selected from halogen, —CN, C1-C2 alkyl, C1-C2 haloalkyl, and —C1-C2 alkoxy. In a still further aspect, Ar¹ is pyridinyl substituted with 0 or 1 group selected from halogen, —CN, C1-C2 alkyl, C1-C2 haloalkyl, and —C1-C2 alkoxy. In yet a further aspect, Ar¹ is pyridinyl monosubstituted with a group selected from halogen, —CN, C1-C2 alkyl, C1-C2 haloalkyl, and —C1-C2 alkoxy. In an even further aspect, Ar¹ is an unsubstituted pyridinyl.

### n. Ar² Groups

In one aspect, Ar² is a 5- to 6-membered aryl or a 5- to 6-membered heteroaryl, and is substituted with 0, 1, 2, or 3 groups independently selected from halogen, —CN, C1-C2 alkyl, and C1-C2 haloalkyl. In a further aspect, Ar² is a 5- to 6-membered aryl or a 5- to 6-membered heteroaryl, and is substituted with 0, 1, or 2 groups independently selected from halogen, —CN, C1-C2 alkyl, and C1-C2 haloalkyl. In a still further aspect, Ar² is a 5- to 6-membered aryl or a 5- to 6-membered heteroaryl, and is substituted with 0 or 1 group selected from halogen, —CN, C1-C2 alkyl, and C1-C2 haloalkyl. In yet a further aspect, Ar² is a 5- to 6-membered aryl or a 5- to 6-membered heteroaryl, and is monosubstituted with a group selected from halogen, —CN, C1-C2 alkyl, and C1-C2 haloalkyl. In an even further aspect, Ar² is a 5- to 6-membered aryl or a 5- to 6-membered heteroaryl, and is unsubstituted.

In various aspects, Ar² is a 5- to 6-membered aryl substituted with 0, 1, 2, or 3 groups independently selected from halogen, —CN, C1-C2 alkyl, and C1-C2 haloalkyl. In a further aspect, Ar² is a 5- to 6-membered aryl substituted with 0, 1, or 2 groups independently selected from halogen, —CN, C1-C2 alkyl, and C1-C2 haloalkyl. In a still further aspect, Ar² is a 5- to 6-membered aryl substituted with 0 or 1 group selected from halogen, —CN, C1-C2 alkyl, and C1-C2 haloalkyl. In yet a further aspect, Ar² is a 5- to 6-membered aryl monosubstituted with a group selected from halogen, —CN, C1-C2 alkyl, and C1-C2 haloalkyl. In an even further aspect, Ar² is an unsubstituted 5- to 6-membered aryl.

In various aspects, Ar² is a 5-membered aryl substituted with 0, 1, 2, or 3 groups independently selected from halogen, —CN, C1-C2 alkyl, and C1-C2 haloalkyl. In a further aspect, Ar² is a 5- to 6-membered aryl substituted with 0, 1, or 2 groups independently selected from halogen, —CN, C1-C2 alkyl, and C1-C2 haloalkyl. In a still further aspect, Ar² is a 5-membered aryl substituted with 0 or 1 group selected from halogen, —CN, C1-C2 alkyl, and C1-C2 haloalkyl. In yet a further aspect, Ar² is a 5-membered aryl monosubstituted with a group selected from halogen, —CN, C1-C2 alkyl, and C1-C2 haloalkyl. In an even further aspect, Ar² is an unsubstituted 5-membered aryl.

In various aspects, Ar² is a 6-membered aryl substituted with 0, 1, 2, or 3 groups independently selected from halogen, —CN, C1-C2 alkyl, and C1-C2 haloalkyl. In a further aspect, Ar² is a 5- to 6-membered aryl substituted with 0, 1, or 2 groups independently selected from halogen, —CN, C1-C2 alkyl, and C1-C2 haloalkyl. In a still further aspect, Ar² is a 6-membered aryl substituted with 0 or 1 group selected from halogen, —CN, C1-C2 alkyl, and C1-C2 haloalkyl. In yet a further aspect, Ar² is a 6-membered aryl monosubstituted with a group selected from halogen, —CN, C1-C2 alkyl, and C1-C2 haloalkyl. In an even further aspect, Ar² is an unsubstituted 6-membered aryl.

In various aspects, Ar² is phenyl substituted with 0, 1, 2, or 3 groups independently selected from halogen, —CN, C1-C2 alkyl, and C1-C2 haloalkyl. In a further aspect, Ar² is phenyl substituted with 0, 1, or 2 groups independently selected from halogen, —CN, C1-C2 alkyl, and C1-C2 haloalkyl. In a still further aspect, Ar² is phenyl substituted with 0 or 1 group selected from halogen, —CN, C1-C2 alkyl, and C1-C2 haloalkyl. In yet a further aspect, Ar² is phenyl monosubstituted with a group selected from halogen, —CN, C1-C2 alkyl, and C1-C2 haloalkyl. In an even further aspect, Ar² is an unsubstituted phenyl.

In various aspects, Ar² is a 5- to 6-membered heteroaryl substituted with 0, 1, 2, or 3 groups independently selected from halogen, —CN, C1-C2 alkyl, and C1-C2 haloalkyl. Examples of 5- or 6-membered heteroaryls include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, and pyrazinyl. In a further aspect, Ar² is a 5- to 6-membered heteroaryl substituted with 0, 1, or 2 groups independently selected from halogen, —CN, C1-C2 alkyl, and C1-C2 haloalkyl. In a still further aspect, Ar² is a 5- to 6-membered heteroaryl substituted with 0 or 1 group selected from halogen, —CN, C1-C2 alkyl, and C1-C2 haloalkyl. In yet a further aspect, Ar² is a 5- to 6-membered heteroaryl monosubstituted with a group selected from halogen, —CN, C1-C2 alkyl, and C1-C2 haloalkyl. In an even further aspect, Ar² is an unsubstituted 5- to 6-membered heteroaryl.

In various aspects, Ar² is a 5-membered heteroaryl substituted with 0, 1, 2, or 3 groups independently selected from halogen, —CN, C1-C2 alkyl, and C1-C2 haloalkyl. In a further aspect, Ar² is a 5-membered heteroaryl substituted with 0, 1, or 2 groups independently selected from halogen, —CN, C1-C2 alkyl, and C1-C2 haloalkyl. In a still further aspect, Ar² is a 5-membered heteroaryl substituted with 0 or 1 group selected from halogen, —CN, C1-C2 alkyl, and C1-C2 haloalkyl. In yet a further aspect, Ar² is a 5-membered heteroaryl monosubstituted with a group selected from halogen, —CN, C1-C2 alkyl, and C1-C2 haloalkyl. In an even further aspect, Ar² is an unsubstituted 5-membered heteroaryl.

In various aspects, Ar² is a 6-membered heteroaryl substituted with 0, 1, 2, or 3 groups independently selected from halogen, —CN, C1-C2 alkyl, and C1-C2 haloalkyl. In a further aspect, Ar² is a 6-membered heteroaryl substituted with 0, 1, or 2 groups independently selected from halogen, —CN, C1-C2 alkyl, and C1-C2 haloalkyl. In a still further aspect, Ar² is a 6-membered heteroaryl substituted with 0 or 1 group selected from halogen, —CN, C1-C2 alkyl, and C1-C2 haloalkyl. In yet a further aspect, Ar² is a 6-membered heteroaryl monosubstituted with a group selected from halogen, —CN, C1-C2 alkyl, and C1-C2 haloalkyl. In an even further aspect, Ar² is an unsubstituted 6-membered heteroaryl.

In various aspects, Ar² is pyridinyl substituted with 0, 1, 2, or 3 groups independently selected from halogen, —CN, C1-C2 alkyl, and C1-C2 haloalkyl. In a further aspect, Ar² is pyridinyl substituted with 0, 1, or 2 groups independently selected from halogen, —CN, C1-C2 alkyl, and C1-C2 haloalkyl. In a still further aspect, Ar² is a 5-membered heteroaryl substituted with 0 or 1 group selected from halogen, —CN, C1-C2 alkyl, and C1-C2 haloalkyl. In yet a further aspect, Ar² is pyridinyl monosubstituted with a group selected from halogen, —CN, C1-C2 alkyl, and C1-C2 haloalkyl. In an even further aspect, Ar² is an unsubstituted pyridinyl.

### o. Cy¹ Groups

In one aspect, Cy¹ is an unsubstituted 9- to 10-membered cycloalkyl group. In a further aspect, Cy¹ is an unsubstituted 9-membered cycloalkyl group such as, for example, 2,3-dihydro-1H-indene and octahydro-1H-indene. In a still further aspect, Cy¹ is an unsubstituted 10-membered cycloalkyl group such as, for example, 1,2,3,4-tetrahydronaphthalene and decahydronaphthalene.

In a further aspect, Cy¹ is a structure:

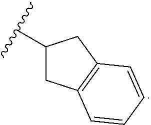 [SMILES: *C1Cc2ccccc2C1]

### 2. Example Compounds

In one aspect, a compound can be present as one or more of the following structures:

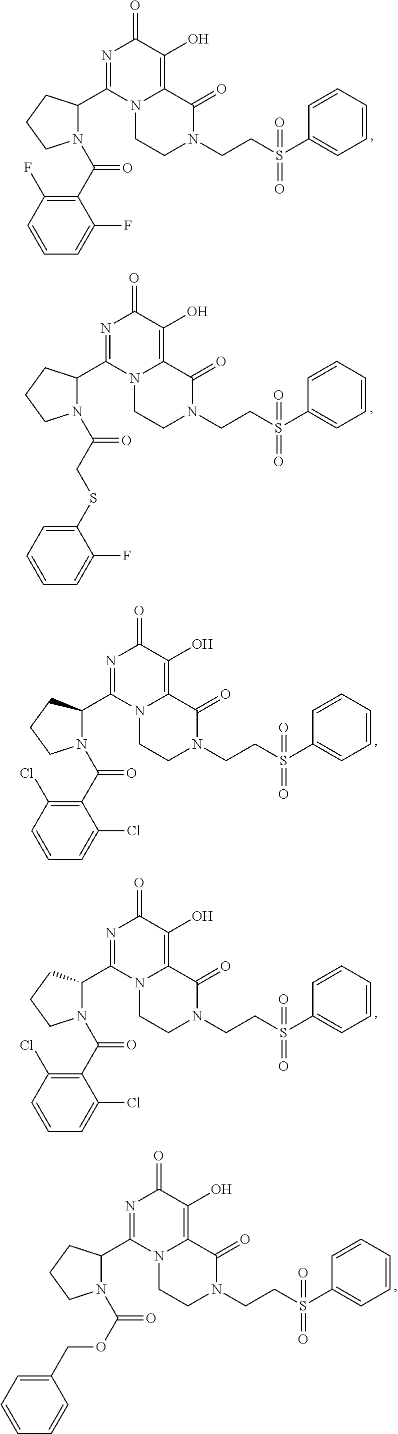 [SMILES: O=C1c2c(O)c(=O)nc(C3CCCN3C(=O)c3c(F)cccc3F)n2CCN1CCS(=O)(=O)c1ccccc1 ; O=C1c2c(O)c(=O)nc(C3CCCN3C(=O)CSc3ccccc3F)n2CCN1CCS(=O)(=O)c1ccccc1 ; O=C1c2c(O)c(=O)nc([C@@H]3CCCN3C(=O)c3c(Cl)cccc3Cl)n2CCN1CCS(=O)(=O)c1ccccc1 ; O=C1c2c(O)c(=O)nc([C@H]3CCCN3C(=O)c3c(Cl)cccc3Cl)n2CCN1CCS(=O)(=O)c1ccccc1 ; O=C1c2c(O)c(=O)nc(C3CCCN3C(=O)OCc3ccccc3)n2CCN1CCS(=O)(=O)c1ccccc1]

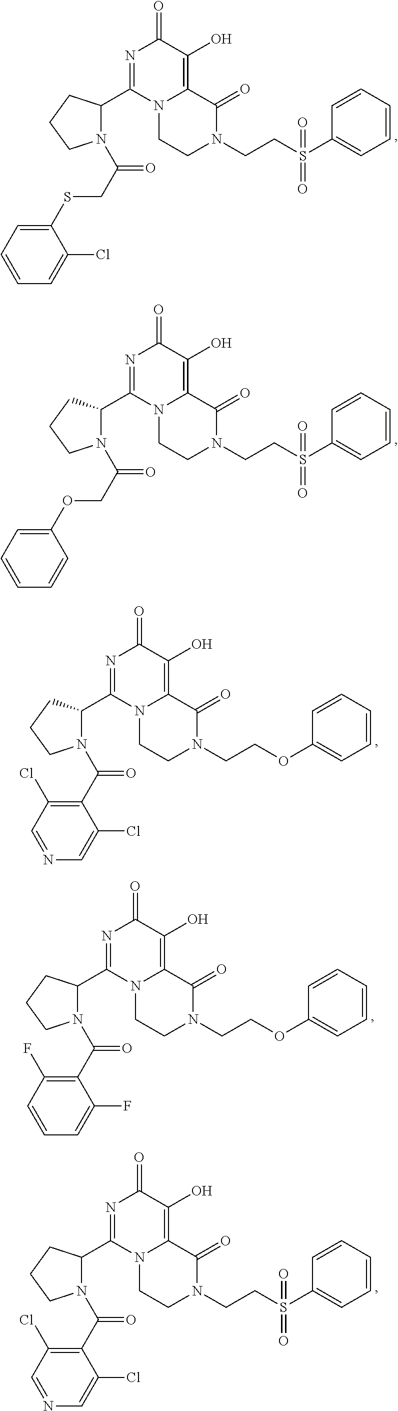 [SMILES: O=C1c2c(O)c(=O)nc(C3CCCN3C(=O)CSc3ccccc3Cl)n2CCN1CCS(=O)(=O)c1ccccc1 ; O=C1c2c(O)c(=O)nc([C@H]3CCCN3C(=O)COc3ccccc3)n2CCN1CCS(=O)(=O)c1ccccc1 ; O=C1c2c(O)c(=O)nc([C@H]3CCCN3C(=O)c3c(Cl)cncc3Cl)n2CCN1CCOc1ccccc1 ; O=C1c2c(O)c(=O)nc(C3CCCN3C(=O)c3c(F)cccc3F)n2CCN1CCOc1ccccc1 ; O=C1c2c(O)c(=O)nc(C3CCCN3C(=O)c3c(Cl)cncc3Cl)n2CCN1CCS(=O)(=O)c1ccccc1]

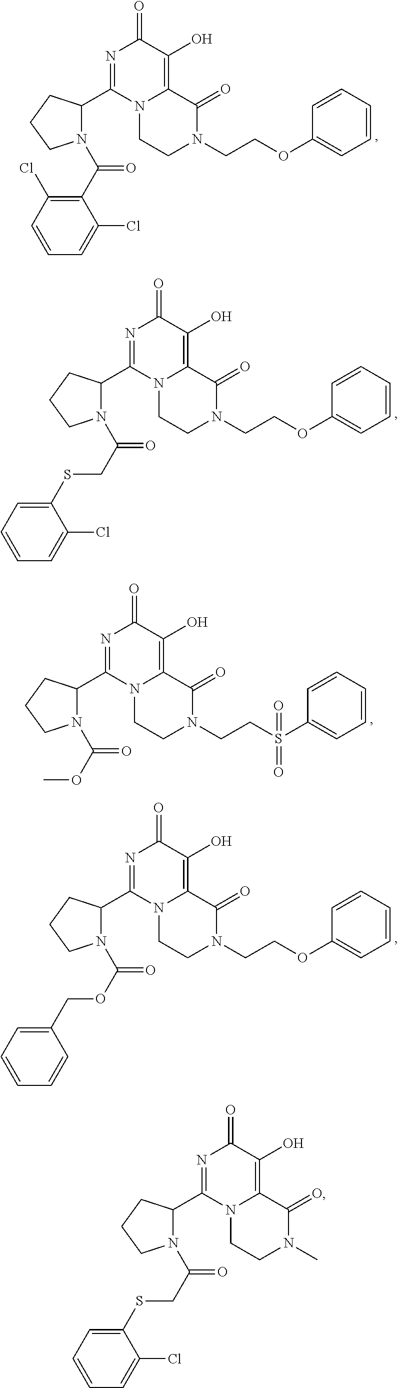 [SMILES: O=C1c2c(O)c(=O)nc(C3CCCN3C(=O)c3c(Cl)cccc3Cl)n2CCN1CCOc1ccccc1 ; O=C1c2c(O)c(=O)nc(C3CCCN3C(=O)CSc3ccccc3Cl)n2CCN1CCOc1ccccc1 ; COC(=O)N1CCCC1c1nc(=O)c(O)c2n1CCN(CCS(=O)(=O)c1ccccc1)C2=O ; O=C1c2c(O)c(=O)nc(C3CCCN3C(=O)OCc3ccccc3)n2CCN1CCOc1ccccc1 ; CN1CCn2c(C3CCCN3C(=O)CSc3ccccc3Cl)nc(=O)c(O)c2C1=O]

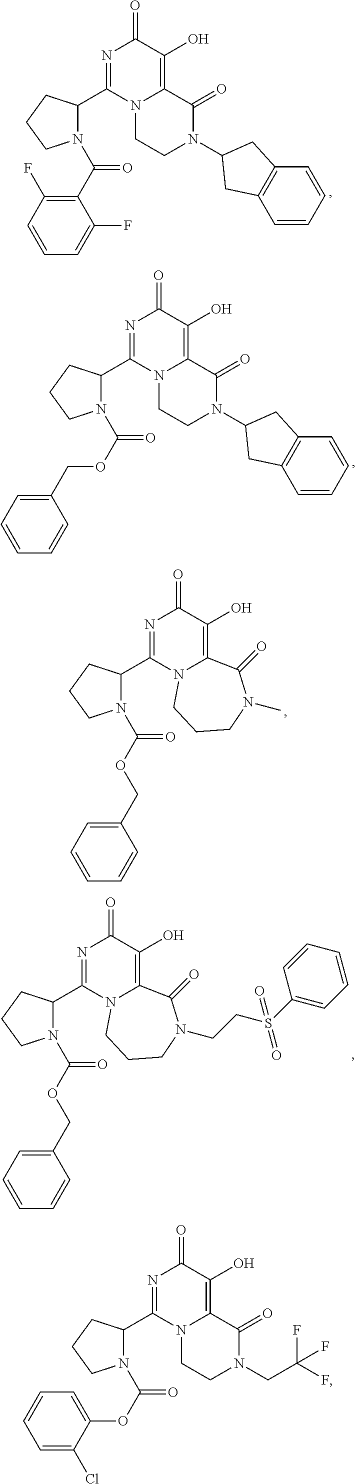

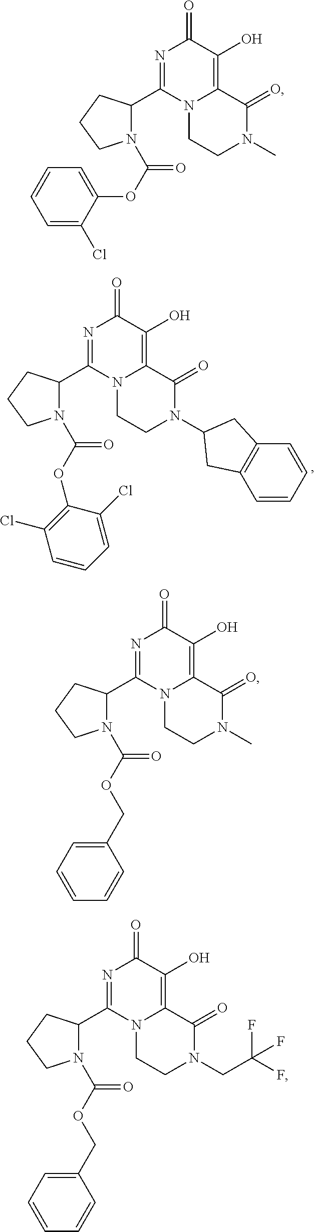

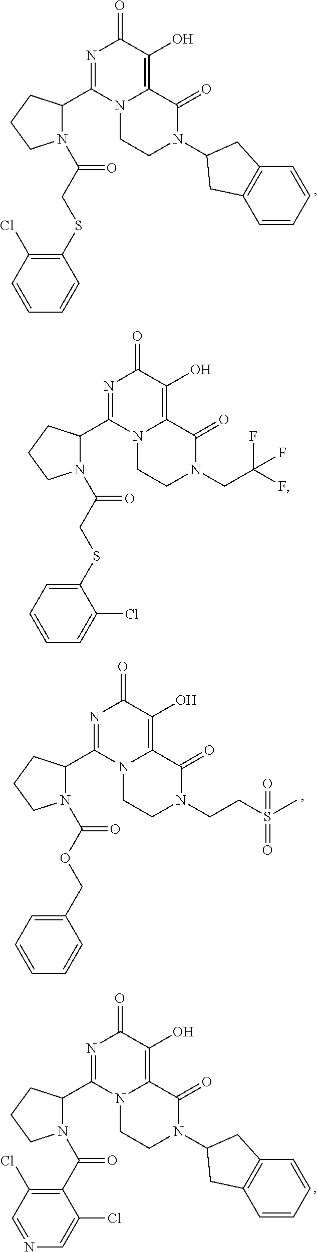 [SMILES: O=C1c2c(O)c(=O)nc(C3CCCN3C(=O)CSc3ccccc3Cl)n2CCN1C1Cc2ccccc2C1 ; O=C1c2c(O)c(=O)nc(C3CCCN3C(=O)CSc3ccccc3Cl)n2CCN1CC(F)(F)F ; CS(=O)(=O)CCN1CCn2c(C3CCCN3C(=O)OCc3ccccc3)nc(=O)c(O)c2C1=O ; O=C1c2c(O)c(=O)nc(C3CCCN3C(=O)c3c(Cl)cncc3Cl)n2CCN1C1Cc2ccccc2C1]

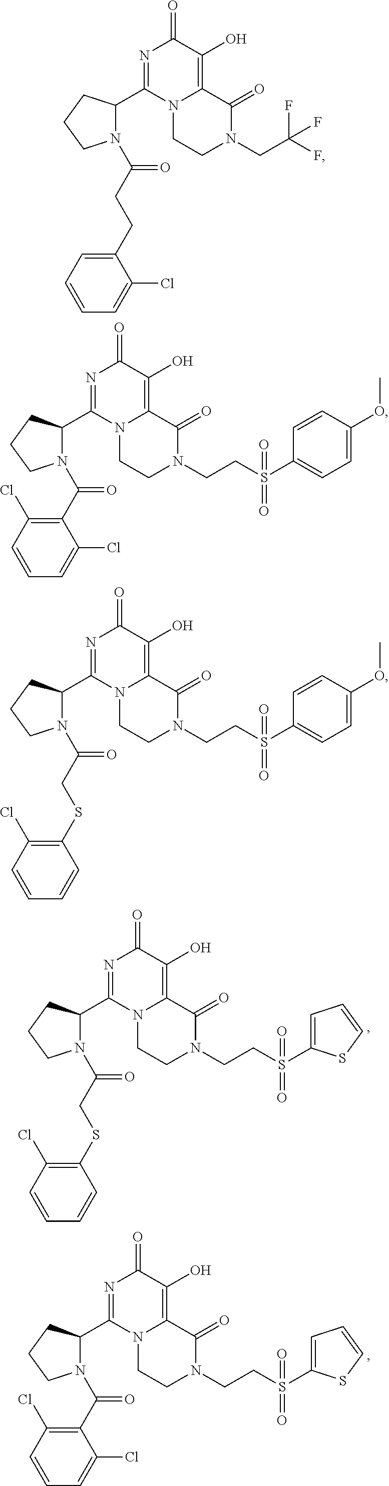

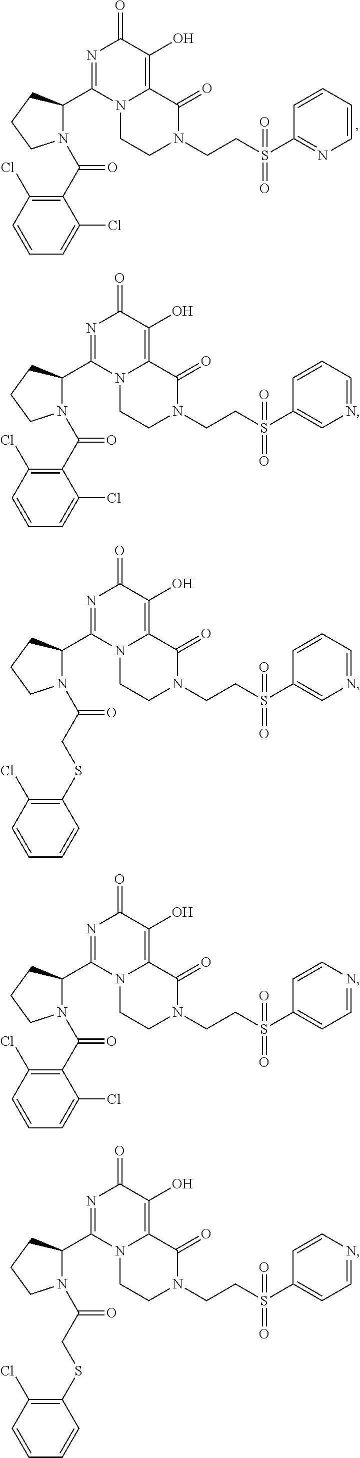 [SMILES: O=C1c2c(O)c(=O)nc([C@@H]3CCCN3C(=O)c3c(Cl)cccc3Cl)n2CCN1CCS(=O)(=O)c1ccccn1 ; O=C1c2c(O)c(=O)nc([C@@H]3CCCN3C(=O)c3c(Cl)cccc3Cl)n2CCN1CCS(=O)(=O)c1cccnc1 ; O=C1c2c(O)c(=O)nc([C@@H]3CCCN3C(=O)CSc3ccccc3Cl)n2CCN1CCS(=O)(=O)c1cccnc1 ; O=C1c2c(O)c(=O)nc([C@@H]3CCCN3C(=O)c3c(Cl)cccc3Cl)n2CCN1CCS(=O)(=O)c1ccncc1 ; O=C1c2c(O)c(=O)nc([C@@H]3CCCN3C(=O)CSc3ccccc3Cl)n2CCN1CCS(=O)(=O)c1ccncc1]

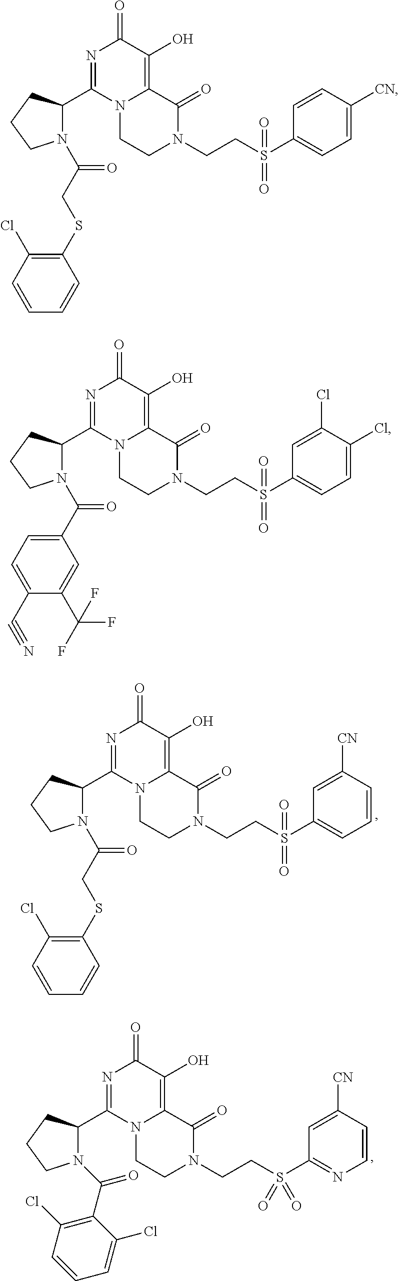 [SMILES: N#Cc1ccc(S(=O)(=O)CCN2CCn3c([C@@H]4CCCN4C(=O)CSc4ccccc4Cl)nc(=O)c(O)c3C2=O)cc1 ; N#Cc1ccc(C(=O)N2CCC[C@H]2c2nc(=O)c(O)c3n2CCN(CCS(=O)(=O)c2ccc(Cl)c(Cl)c2)C3=O)cc1C(F)(F)F ; N#Cc1cccc(S(=O)(=O)CCN2CCn3c([C@@H]4CCCN4C(=O)CSc4ccccc4Cl)nc(=O)c(O)c3C2=O)c1 ; N#Cc1ccnc(S(=O)(=O)CCN2CCn3c([C@@H]4CCCN4C(=O)c4c(Cl)cccc4Cl)nc(=O)c(O)c3C2=O)c1]

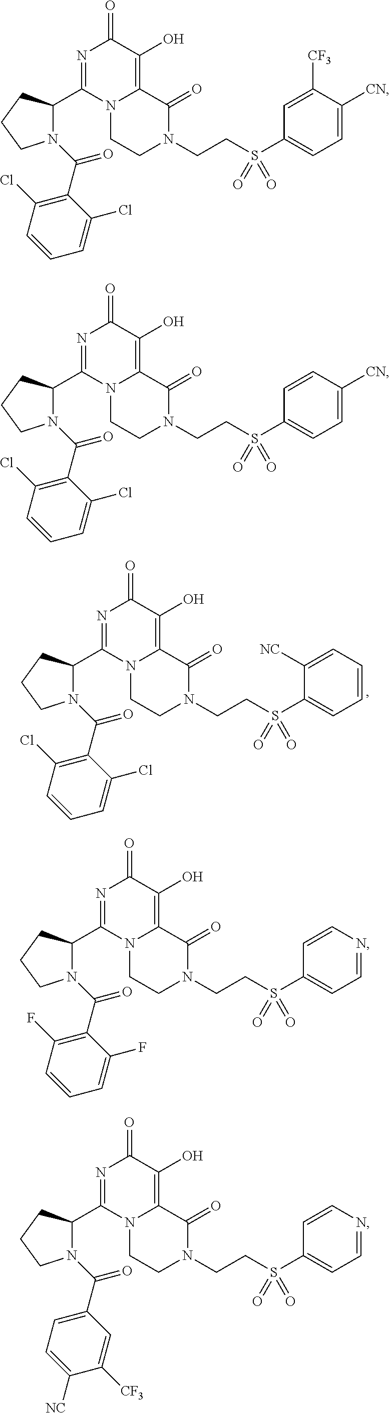

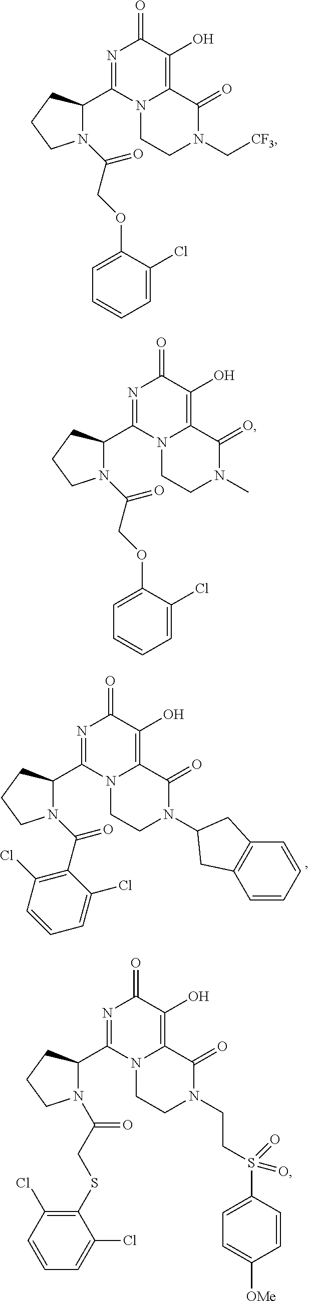

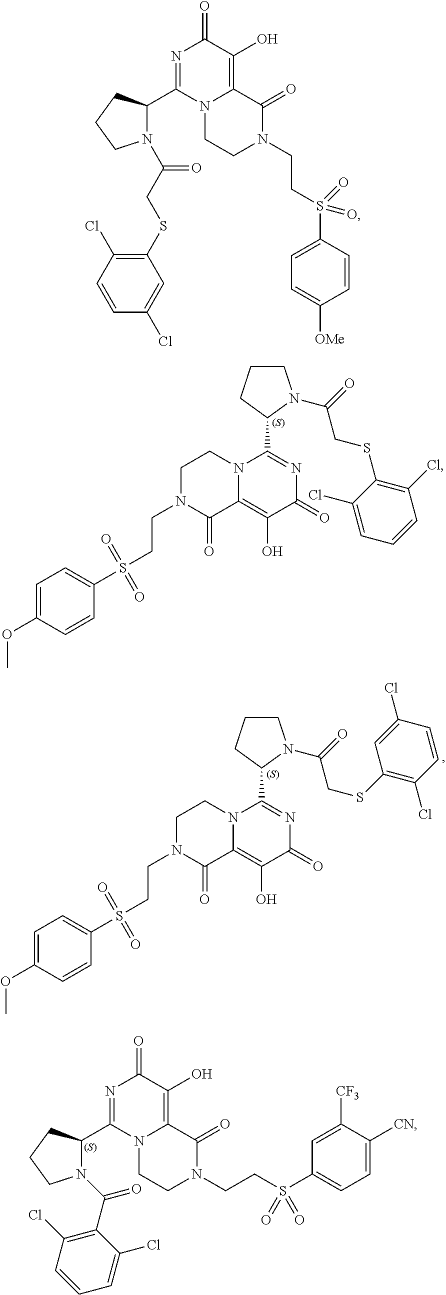

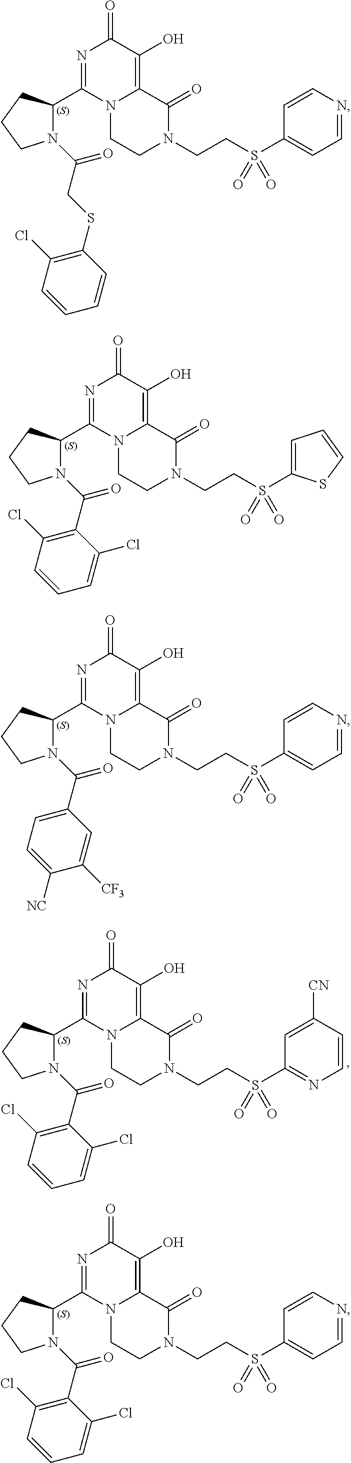

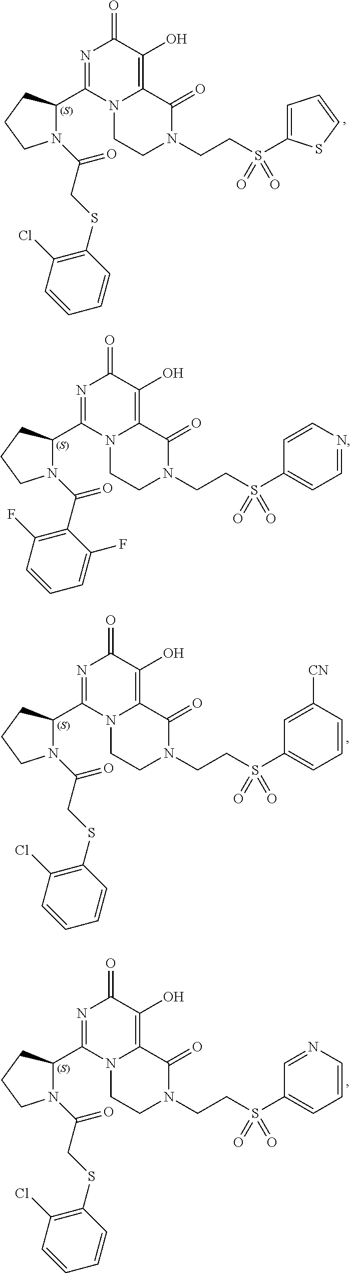

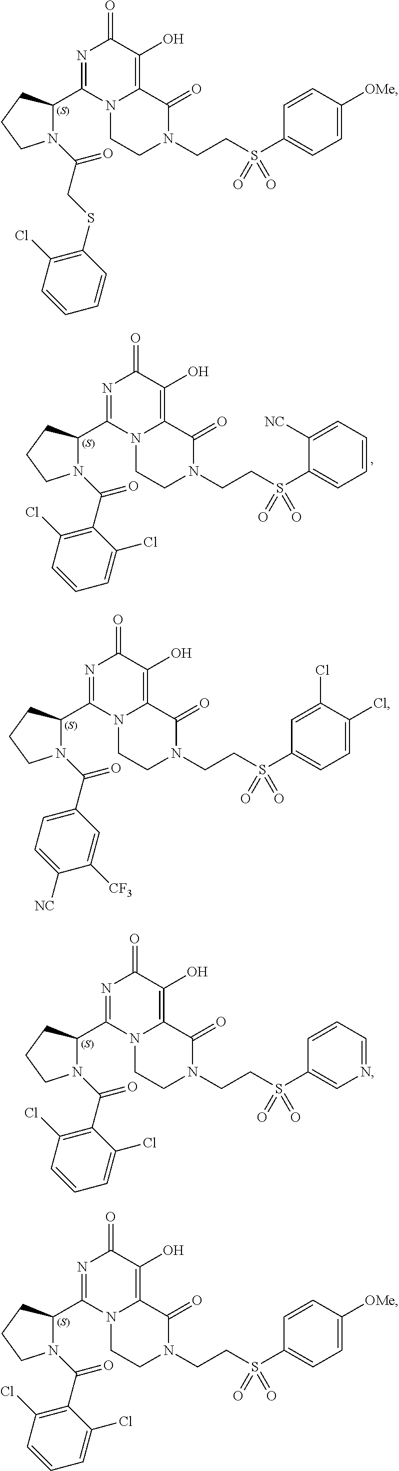

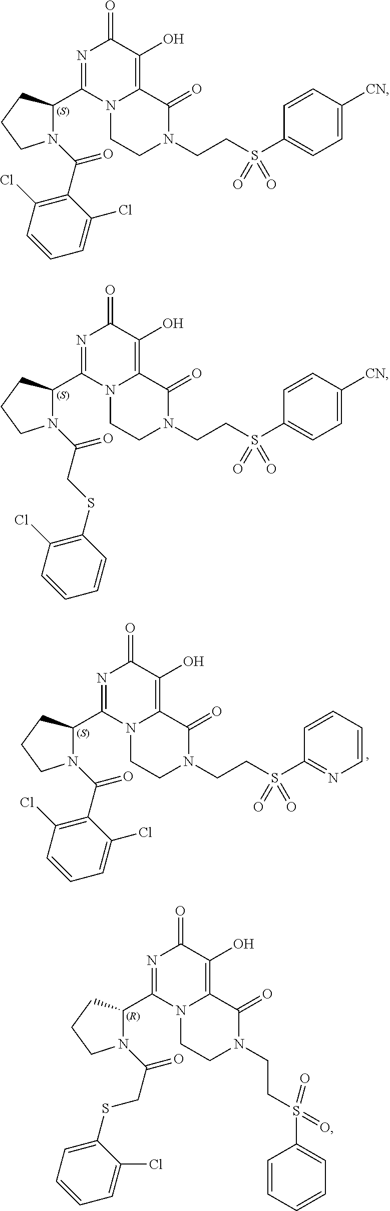

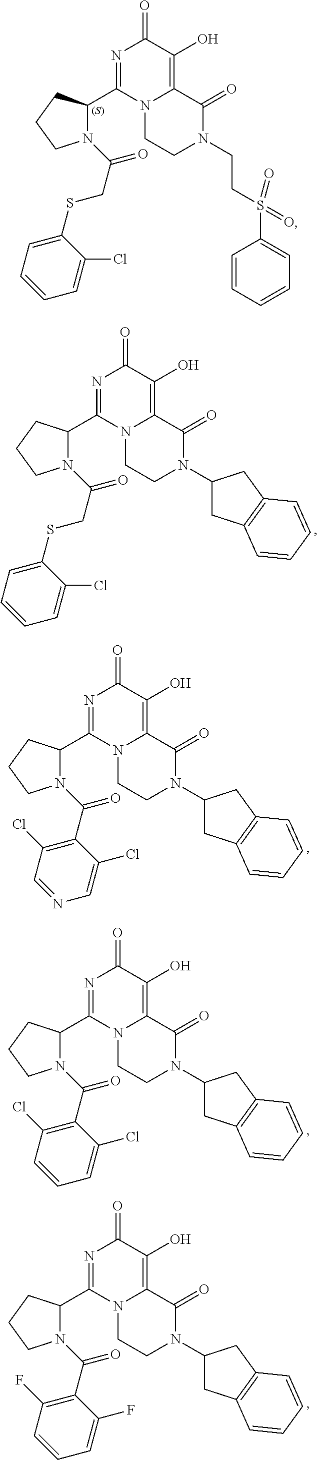

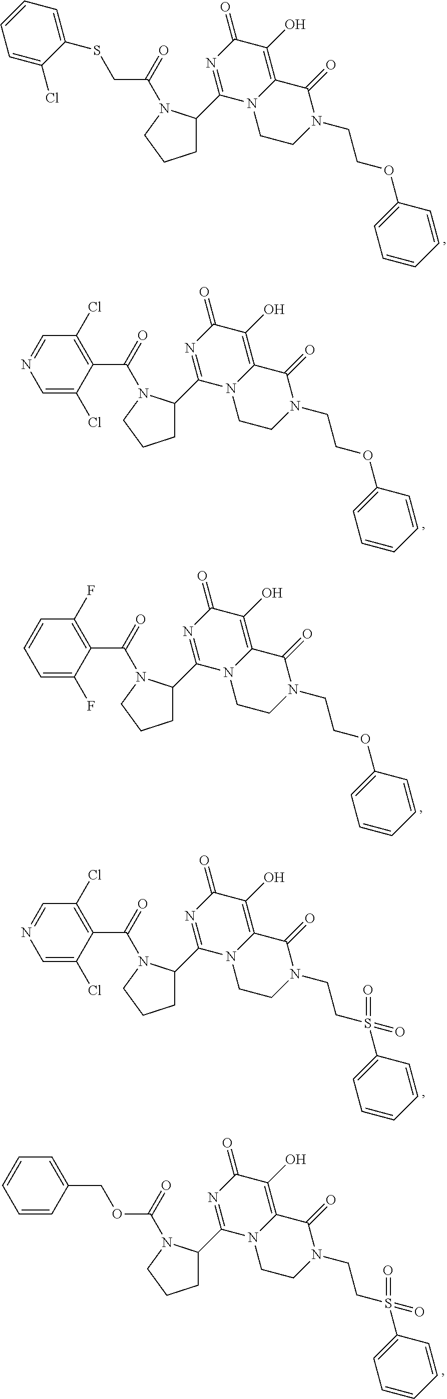 [SMILES: O=C1c2c(O)c(=O)nc(C3CCCN3C(=O)CSc3ccccc3Cl)n2CCN1CCOc1ccccc1 ; O=C1c2c(O)c(=O)nc(C3CCCN3C(=O)c3c(Cl)cncc3Cl)n2CCN1CCOc1ccccc1 ; O=C1c2c(O)c(=O)nc(C3CCCN3C(=O)c3c(F)cccc3F)n2CCN1CCOc1ccccc1 ; O=C1c2c(O)c(=O)nc(C3CCCN3C(=O)c3c(Cl)cncc3Cl)n2CCN1CCS(=O)(=O)c1ccccc1 ; O=C1c2c(O)c(=O)nc(C3CCCN3C(=O)OCc3ccccc3)n2CCN1CCS(=O)(=O)c1ccccc1]

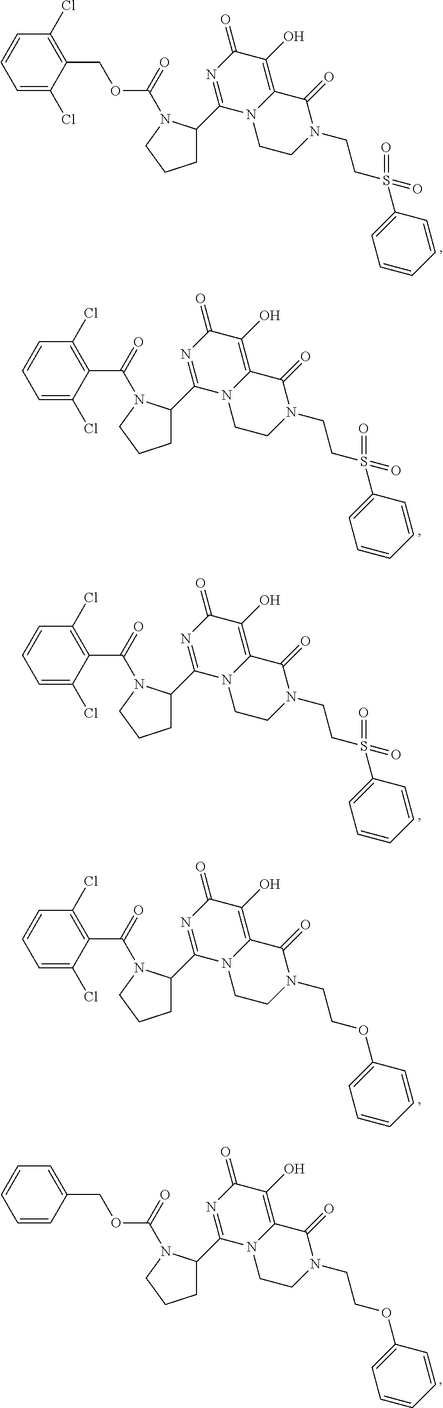 [SMILES: O=C1c2c(O)c(=O)nc(C3CCCN3C(=O)OCc3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCOc1ccccc1 ; O=C1c2c(O)c(=O)nc(C3CCCN3C(=O)OCc3ccccc3)n2CCN1CCOc1ccccc1]

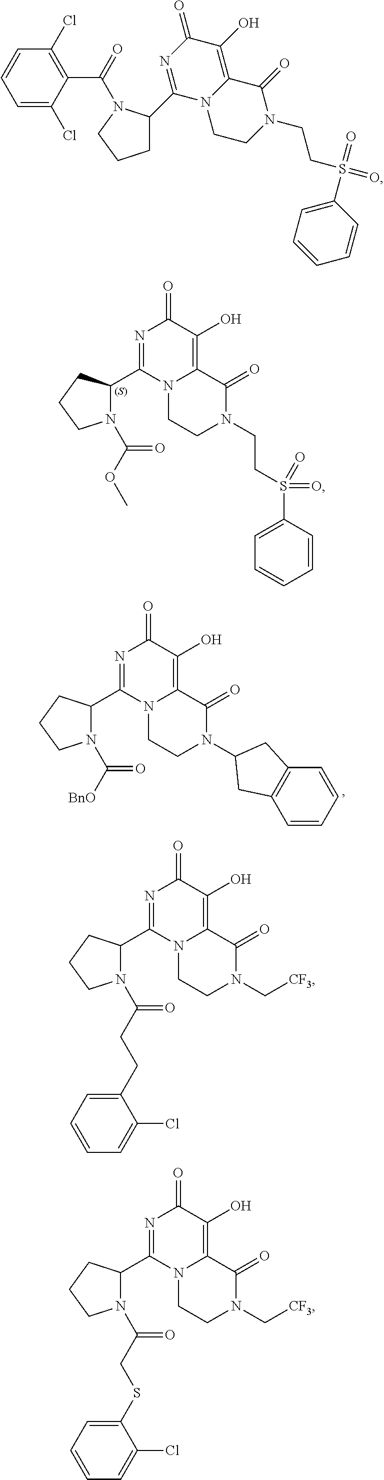

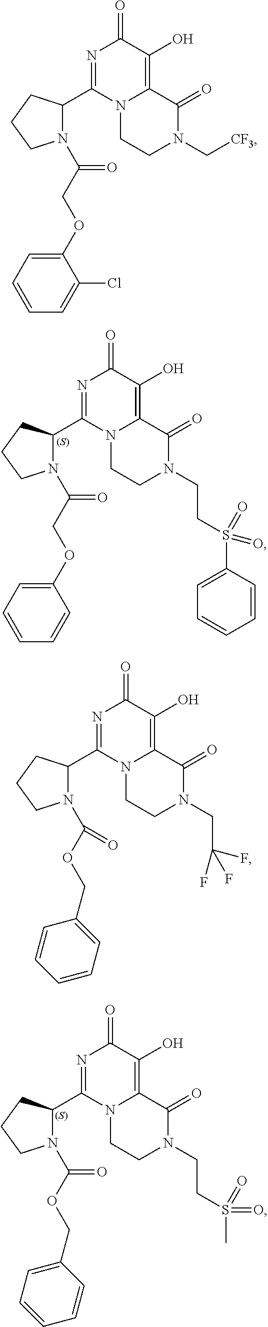

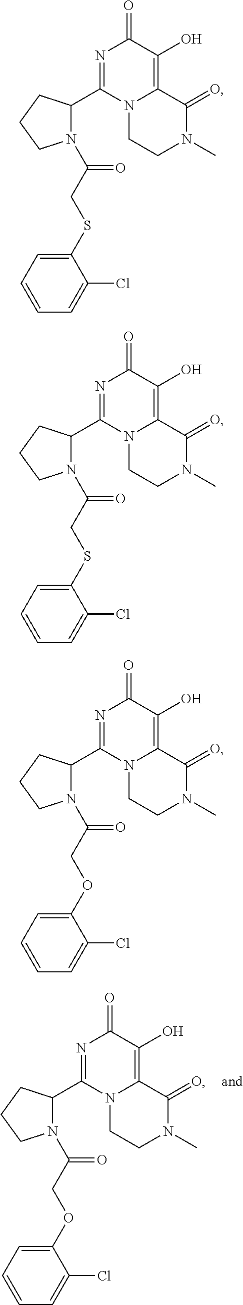 [SMILES: CN1CCn2c(C3CCCN3C(=O)CSc3ccccc3Cl)nc(=O)c(O)c2C1=O ; CN1CCn2c(C3CCCN3C(=O)CSc3ccccc3Cl)nc(=O)c(O)c2C1=O ; CN1CCn2c(C3CCCN3C(=O)COc3ccccc3Cl)nc(=O)c(O)c2C1=O ; CN1CCn2c(C3CCCN3C(=O)COc3ccccc3Cl)nc(=O)c(O)c2C1=O ; "and"]

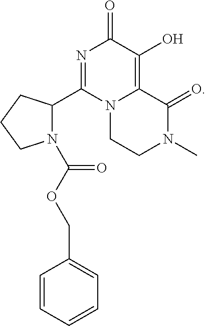 [SMILES: CN1CCn2c(C3CCCN3C(=O)OCc3ccccc3)nc(=O)c(O)c2C1=O]

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. Pharmaceutical Compositions

In one aspect, disclosed are pharmaceutical compositions comprising a disclosed compound, or a pharmaceutically acceptable salt thereof, and a pharmaceutically acceptable carrier.

Thus, in one aspect, disclosed are pharmaceutical compositions comprising a therapeutically effective amount of a compound having a structure represented by a formula:

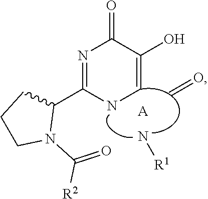

wherein: A is a 6-7 membered heterocycle; R¹ is C1-C3 alkyl, C1-C3 alkoxy, C1-C3 haloalkyl, C1-C3 alkylsulfonyl, or a 9- to 10-membered cycloaryl, wherein R¹ can further be independently substituted with one or more R^{x} groups; R^{x} is sulfonyl, oxo, C1-C2 haloalkyl, 5- to 6-membered aryl, or 5- to 6-membered heteroaryl, wherein R^{x} can independently further be substituted with one or more R^{a} groups; R^{a} is C1-C2 alkyl, 5- to 6-membered aryl, 5- to 6-membered heteroaryl, or C1-C2 haloalkyl, wherein R^{a} can independently be substituted with one or more R^{a1} groups; R^{a1} is halo, C1-C2 alkoxy, cyano, or C1-C2 haloalkyl; R² is C1-C2 alkyl, 5- to 6-membered aryl, oxo, 5- to 6-membered heteroaryl, or C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further be substituted with one or more R^{b} groups; R^{b} is halo or 5- to 6-membered aryl; R^{b1} is halo; and wherein the wavy line indicates either R or S enantiomer at that bond, or a pharmaceutically acceptable salt or hydrate thereof, and a pharmaceutically acceptable carrier.

In one aspect, disclosed are pharmaceutical compositions comprising a therapeutically effective amount of a compound having a structure represented by a formula:

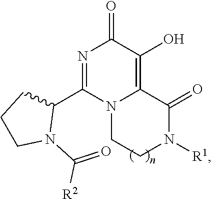

wherein: n is 1 or 2; R¹ is C1-C3 alkyl, C1-C3 haloalkyl, —(C1-C3 alkyl)OR¹⁰, —(C1-C3 alkyl)SO₂R¹⁰, or Cy¹; R¹⁰ is C1-C2 alkyl or Ar¹; Ar¹ is a 5- to 6-membered aryl or a 5- to 6-membered heteroaryl, and is substituted with 0, 1, 2, or 3 groups independently selected from halogen, —CN, C1-C2 alkyl, C1-C2 haloalkyl, and —C1-C2 alkoxy; Cy¹ is an unsubstituted 9- to 10-membered cycloalkyl group; R² is C1-C2 alkyl, —(C1-C2 alkyl)Ar², —O(C1-C2 alkyl), —O(C1-C2 alkyl)Ar², —(C1-C2 alkyl)OAr², —S(C1-C2 alkyl), —S(C1-C2 alkyl)Ar², —(C1-C2 alkyl)SAr², or Ar²; and Ar² is a 5- to 6-membered aryl or a 5- to 6-membered heteroaryl, and is substituted with 0, 1, 2, or 3 groups independently selected from halogen, —CN, C1-C2 alkyl, and C1-C2 haloalkyl, or a pharmaceutically acceptable salt thereof, and a pharmaceutically acceptable carrier.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, P A 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, the pharmaceutical composition is administered to a mammal. In a still further aspect, the mammal is a human. In an even further aspect, the human is a patient.

In a further aspect, the pharmaceutical composition is used to treat a viral infection such as, for example, influenza.

It is understood that the disclosed compositions can be prepared from the disclosed compounds. It is also understood that the disclosed compositions can be employed in the disclosed methods of using.

### D. Methods of Making Compounds

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-II, as described and exemplified below. The following examples are provided so that the invention might be more fully understood, are illustrative only, and should not be construed as limiting.

### 1. Route I

In one aspect, the disclosed compounds can be prepared as shown below.

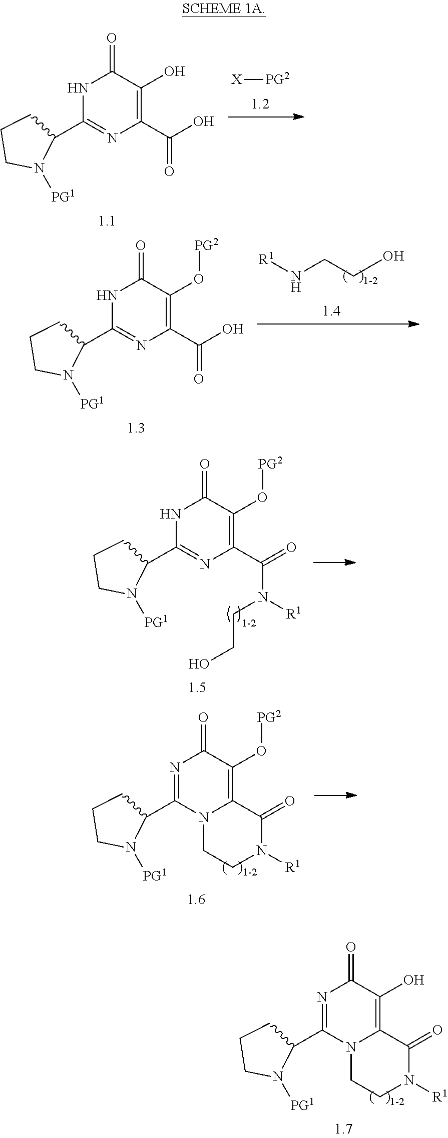

Compounds are represented in generic form, wherein PG¹ is an amine protecting group, PG² is a hydroxyl protecting group, and X is a halogen, and with other substituents as noted in compound descriptions elsewhere herein. A more specific example is set forth below.

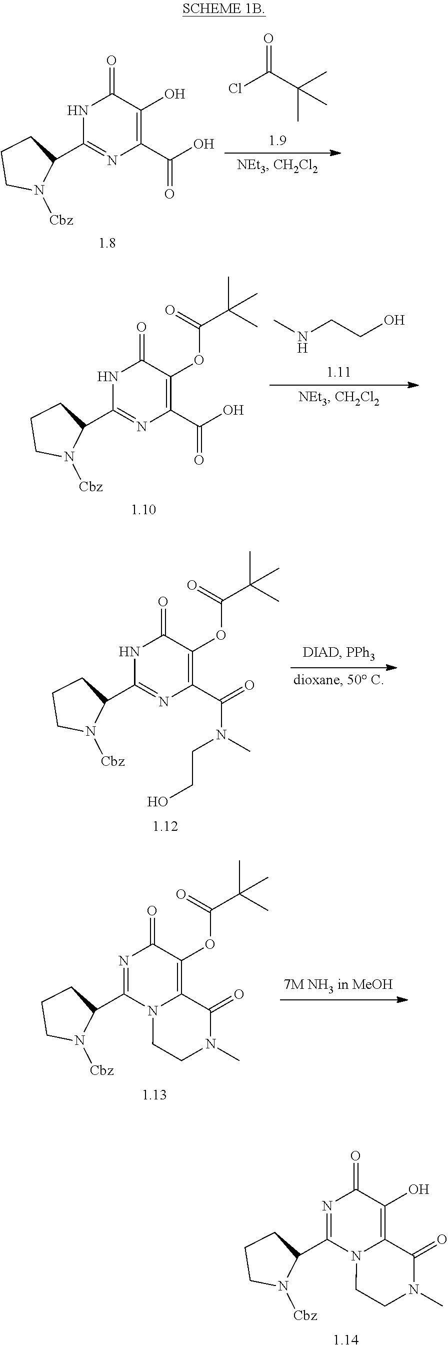

In one aspect, compounds of type 1.14, and similar compounds, can be prepared according to reaction Scheme 1B above. Thus, compounds of type 1.10 can be prepared by protecting an appropriate alcohol, e.g., 1.8 as shown above, using an appropriate halide. Appropriate halides are commercially available or prepared by methods known to one skilled in the art. The protection is carried out in the presence of an appropriate base, e.g., triethyl amine, in an appropriate solvent, e.g., dichloromethane. Compounds of type 1.12 can be prepared by a coupling reaction between an appropriate carboxylic acid, e.g., 1.10 as shown above, and an appropriate amine, e.g., 1.11 as shown above. Appropriate amines are commercially available or prepared by one of skill in the art. The coupling reaction is carried out in the presence of an appropriate base, e.g., triethyl amine, in an appropriate solvent, e.g., dichloromethane. Compounds of type 1.13 can be prepared by an intramolecular cyclization (e.g., a Mitsonobu reaction) of an appropriate hydroxyl amine, e.g., 1.12 as shown above. Intramolecular cyclizations can be carried out in the presence of an appropriate activating agent, e.g., an azodicarboxylate such as diisopropyl azodicarboxylate (DIAD), and an appropriate nucleophile, e.g., triphenylphosphine, in an appropriate solvent, e.g., dioxane, at an appropriate temperature, e.g., 50° C. Compounds of type 1.14 can be prepared by deprotection of an appropriate tricycle analog, e.g., 1.13 as shown above. The deprotection can be carried out in the presence of an appropriate base, e.g., 7M ammonia, in an appropriate solvent, e.g., methanol. As can be appreciated by one skilled in the art, the above reaction provides an example of a generalized approach wherein compounds similar in structure to the specific reactants above (compounds similar to compounds of type 1.1, 1.2, 1.3, 1.4, 1.5, and 1.6), can be substituted in the reaction to provide substituted tricycle analogs similar to Formula 1.7.

### 2. Route II

In one aspect, the disclosed compounds can be prepared as shown below.

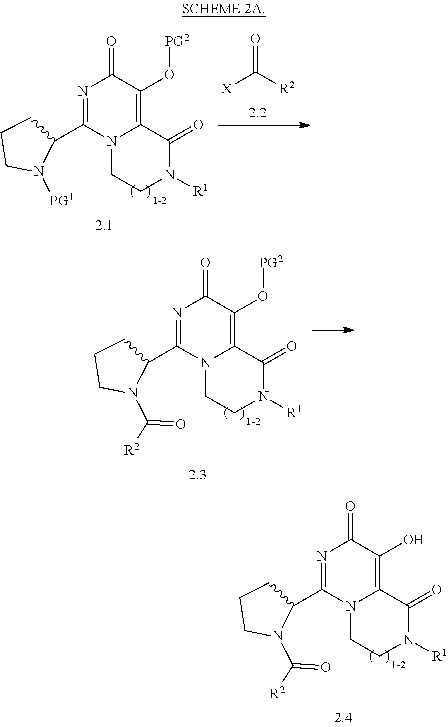

Compounds are represented in generic form, wherein PG¹ is an amine protecting group, PG² is a hydroxyl protecting group, and X is a halogen, and with other substituents as noted in compound descriptions elsewhere herein. A more specific example is set forth below.

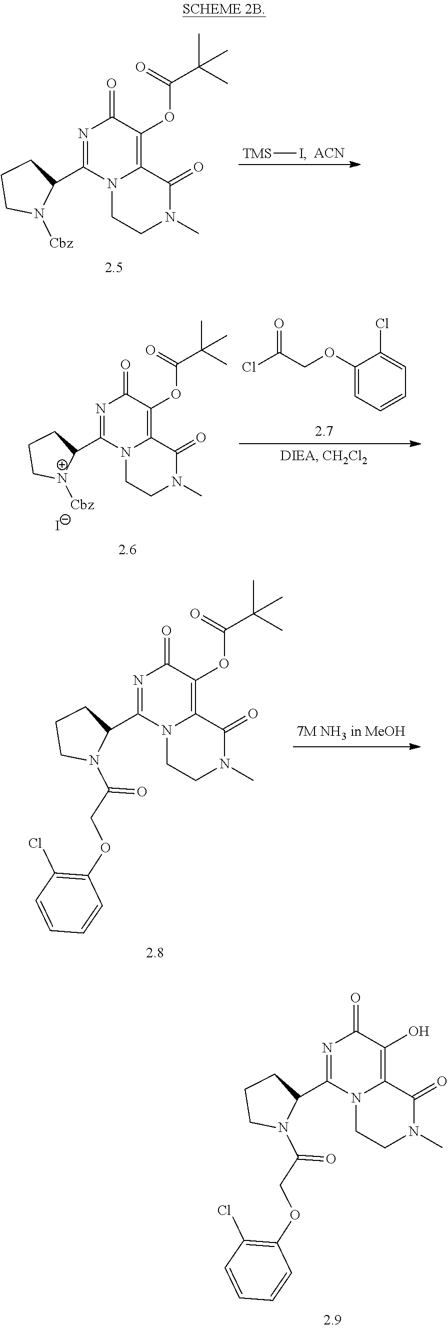

In one aspect, compounds of type 2.9, and similar compounds, can be prepared according to reaction Scheme 2B above. Thus, compounds of type 2.6 can be prepared by deprotecting an appropriate amine, e.g., 2.5 as shown above. The deprotection is carried out in the presence of an appropriate halide, e.g., trimethylsilyl iodide, in an appropriate solvent, e.g., acetonitrile. Compounds of type 2.8 can be prepared by a coupling reaction between an appropriate amine, e.g., 2.6 as shown above, and an appropriate acid halide, e.g., 2.7 as shown above. Appropriate acide halids are commercially available or prepared by one of skill in the art. The coupling reaction is carried out in the presence of an appropriate base, e.g., N,N-diisopropylethylamine (DIPEA), in an appropriate solvent, e.g., dichloromethane. Compounds of type 2.9 can be prepared by deprotection of an appropriate tricycle analog, e.g., 2.8 as shown above. The deprotection can be carried out in the presence of an appropriate base, e.g., 7M ammonia, in an appropriate solvent, e.g., methanol. As can be appreciated by one skilled in the art, the above reaction provides an example of a generalized approach wherein compounds similar in structure to the specific reactants above (compounds similar to compounds of type 2.1, 2.2, 2.3, and 2.4), can be substituted in the reaction to provide substituted tricycle analogs similar to Formula 2.5.

### E. Treating a Viral Infection

In one aspect, disclosed are methods of treating a viral infection in a subject, the method comprising the step of administering to the subject an effective amount of at least one disclosed compound, or a pharmaceutically acceptable salt thereof.

Thus, in one aspect, disclosed are methods for treating a viral infection in a subject, the method comprising administering to the subject an effective amount of a compound having a structure represented by a formula:

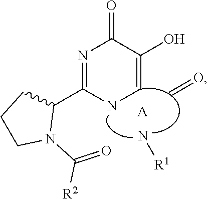

wherein: A is a 6-7 membered heterocycle; R¹ is C1-C3 alkyl, C1-C3 alkoxy, C1-C3 haloalkyl, C1-C3 alkylsulfonyl, or a 9- to 10-membered cycloaryl, wherein R¹ can further be independently substituted with one or more R^{x} groups; R^{x} is sulfonyl, oxo, C1-C2 haloalkyl, 5- to 6-membered aryl, or 5- to 6-membered heteroaryl, wherein R^{x} can independently further be substituted with one or more R^{a} groups; R^{a} is C1-C2 alkyl, 5- to 6-membered aryl, 5- to 6-membered heteroaryl, or C1-C2 haloalkyl, wherein R^{a} can independently be substituted with one or more R^{a1} groups; R^{a1} is halo, C1-C2 alkoxy, cyano, or C1-C2 haloalkyl; R² is C1-C2 alkyl, 5- to 6-membered aryl, oxo, 5- to 6-membered heteroaryl, or C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further be substituted with one or more R^{b} groups; R^{b} is halo or 5- to 6-membered aryl; R^{b1} is halo; and wherein the wavy line indicates either R or S enantiomer at that bond, or a pharmaceutically acceptable salt or hydrate thereof, thereby treating the viral infection.

In one aspect, disclosed are methods for treating a viral infection in a subject, the method comprising administering to the subject an effective amount of a compound having a structure represented by a formula:

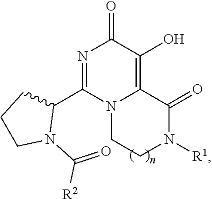

wherein: n is 1 or 2; R¹ is C1-C3 alkyl, C1-C3 haloalkyl, —(C1-C3 alkyl)OR¹⁰, —(C1-C3 alkyl)SO₂R¹⁰, or Cy¹; R¹⁰ is C1-C2 alkyl or Ar¹; Ar¹ is a 5- to 6-membered aryl or a 5- to 6-membered heteroaryl, and is substituted with 0, 1, 2, or 3 groups independently selected from halogen, —CN, C1-C2 alkyl, C1-C2 haloalkyl, and —C1-C2 alkoxy; Cy¹ is an unsubstituted 9- to 10-membered cycloalkyl group; R² is C1-C2 alkyl, —(C1-C2 alkyl)Ar², —O(C1-C2 alkyl), —O(C1-C2 alkyl)Ar², —(C1-C2 alkyl)OAr², —S(C1-C2 alkyl), —S(C1-C2 alkyl)Ar², —(C1-C2 alkyl)SAr², or Ar²; and Ar² is a 5- to 6-membered aryl or a 5- to 6-membered heteroaryl, and is substituted with 0, 1, 2, or 3 groups independently selected from halogen, —CN, C1-C2 alkyl, and C1-C2 haloalkyl, or a pharmaceutically acceptable salt thereof, thereby treating the viral infection.

In a further aspect, the subject has been diagnosed with a need for treatment of the viral infection prior to the administering step.

In a further aspect, the subject is a mammal. In a still further aspect, the mammal is a human.

In a further aspect, the method further comprises the step of identifying a subject in need of treatment of the viral infection.

In a further aspect, the viral infection is selected from human immunodeficiency virus (HIV), human papillomavirus (HPV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, yellow fever virus, tick-bome encephalitis virus, Chikungunya virus (CHIKV), Venezuelan equine encephalitis (VEEV), Eastern equine encephalitis (EEEV), Western equine encephalitis (WEEV), dengue (DENV), influenza, West Nile virus (WNV), zika (ZIKV), Middle East Respiratory Syndromes (MERS), Severe Acute Respiratory Syndrome (SARS), coronavirus disease 2019 (COVID-19), and influenza virus. In a still further aspect, the viral infection is influenza virus.

In a further aspect, the effective amount is a therapeutically effective amount. In a still further aspect, the effective amount is a prophylactically effective amount.

In a further aspect, the method further comprises the step of administering a therapeutically effective amount of at least one agent associated with the treatment of a viral infection such as, for example, an antiviral agent. Examples of antiviral agents include, but are not limited to, acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, MK-4482 (EIDD-2801), penciclovir, raltegravir, ribavirin, rimantadine hydrochloride, rilpivirine (TMC-278), remdesivir, saquinavir mesylate, SCH—C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

### F. Additional Methods of Using the Compounds

The compounds and pharmaceutical compositions of the invention are useful in treating or controlling viral infections due to, for example, an Alphavirus (e.g., Chikungunya virus (CHIKV), Ross River virus, Venezuelan equine encephalitis (VEEV), Eastern equine encephalitis (EEEV), and Western equine encephalitis (WEEV)), a Flavivirus (e.g., dengue virus (DENV), West Nile virus (WNV), zika virus (ZIKV), tick-borne encephalitis virus, and yellow fever virus), a Coronavirus (e.g., Middle East Respiratory Syndromes coronavirus (MERS-CoV), Severe Acute Respiratory Syndrome coronavirus (SARS-CoV), and SARS-CoV-2), and a influenza virus (influenza A and influenza B). Additional examples of viral infections for which the compounds and compositions can be useful in treating, include, but are not limited to, human immunodeficiency virus (HIV), human papillomavirus (HPV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, yellow fever virus, tick-borne encephalitis virus, Chikungunya virus (CHIKV), Venezuelan equine encephalitis (VEEV), Eastern equine encephalitis (EEEV), Western equine encephalitis (WEEV), dengue (DENV), influenza, West Nile virus (WNV), zika (ZIKV), Middle East Respiratory Syndromes (MERS), Severe Acute Respiratory Syndrome (SARS), coronavirus disease 2019 (COVID-19), and influenza virus.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a viral infection, such as, for example, influenza.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a viral infection, such as, for example, influenza.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Use of Compounds

In one aspect, the invention relates to the use of a disclosed compound or a product of a disclosed method. In a further aspect, a use relates to the manufacture of a medicament for the treatment of a viral infection in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a viral infection in a subject. In one aspect, the use is characterized in that the subject is a human. In one aspect, the use is characterized in that the viral infection is influenza.

In a further aspect, the use relates to the manufacture of a medicament for the treatment of a viral infection in a subject.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a viral infection in a mammal. In a further aspect, the viral infection is influenza.

### 2. Manufacture of a Medicament

In one aspect, the invention relates to a method for the manufacture of a medicament for treating a viral infection in a subject having the disorder, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a viral infection. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 3. Kits

In one aspect, disclosed are kits comprising an effective amount of a disclosed compound, and one or more of: (a) an antiviral agent; (b) an immunity booster; (c) instructions for administering the compound in connection with treating a viral infection; (d) instructions for administering the compound in connection with reducing the risk of viral infection; and (e) instructions for treating a viral infection.

Thus, in one aspect, disclosed are kits comprising an effective amount of a compound having a structure represented by a formula:

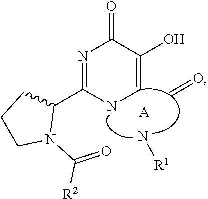

wherein: A is a 6-7 membered heterocycle; R¹ is C1-C3 alkyl, C1-C3 alkoxy, C1-C3 haloalkyl, C1-C3 alkylsulfonyl, or a 9- to 10-membered cycloaryl, wherein R¹ can further be independently substituted with one or more R^{x} groups; R^{x} is sulfonyl, oxo, C1-C2 haloalkyl, 5- to 6-membered aryl, or 5- to 6-membered heteroaryl, wherein R^{x} can independently further be substituted with one or more R^{a} groups; R^{a} is C1-C2 alkyl, 5- to 6-membered aryl, 5- to 6-membered heteroaryl, or C1-C2 haloalkyl, wherein R^{a} can independently be substituted with one or more R^{a1} groups; R^{a1} is halo, C1-C2 alkoxy, cyano, or C1-C2 haloalkyl; R² is C1-C2 alkyl, 5- to 6-membered aryl, oxo, 5- to 6-membered heteroaryl, or C1-C2 alkoxy, wherein R² can further be independently substituted with one or more R^{y} groups; R^{y} is halo, oxo, C1-C2 alkyl, sulfidyl, cyano, C1-C2 haloalkyl, or 5- to 6-membered aryl, wherein R^{y} can independently further be substituted with one or more R^{b} groups; R^{b} is halo or 5- to 6-membered aryl; R^{b1} is halo; and wherein the wavy line indicates either R or S enantiomer at that bond, or a pharmaceutically acceptable salt or hydrate thereof, and one or more of (a) an antiviral agent; (b) an immunity booster; (c) instructions for administering the compound in connection with treating a viral infection; (d) instructions for administering the compound in connection with reducing the risk of viral infection; and (e) instructions for treating a viral infection.

In one aspect, disclosed are kits comprising an effective amount of a compound having a structure represented by a formula:

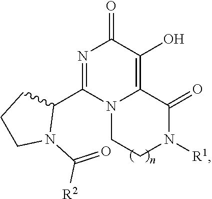

wherein: n is 1 or 2; R¹ is C1-C3 alkyl, C1-C3 haloalkyl, —(C1-C3 alkyl)OR¹⁰, —(C1-C3 alkyl)SO₂R¹⁰, or Cy¹; R¹⁰ is C1-C2 alkyl or Ar¹; Ar¹ is a 5- to 6-membered aryl or a 5- to 6-membered heteroaryl, and is substituted with 0, 1, 2, or 3 groups independently selected from halogen, —CN, C1-C2 alkyl, C1-C2 haloalkyl, and —C1-C2 alkoxy; Cy¹ is an unsubstituted 9- to 10-membered cycloalkyl group; R² is C1-C2 alkyl, —(C1-C2 alkyl)Ar², —O(C1-C2 alkyl), —O(C1-C2 alkyl)Ar², —(C1-C2 alkyl)OAr², —S(C1-C2 alkyl), —S(C1-C2 alkyl)Ar², —(C1-C2 alkyl)SAr², or Ar²; and Ar² is a 5- to 6-membered aryl or a 5- to 6-membered heteroaryl, and is substituted with 0, 1, 2, or 3 groups independently selected from halogen, —CN, C1-C2 alkyl, and C1-C2 haloalkyl, or a pharmaceutically acceptable salt thereof, and one or more of (a) an antiviral agent; (b) an immunity booster; (c) instructions for administering the compound in connection with treating a viral infection; (d) instructions for administering the compound in connection with reducing the risk of viral infection; and (e) instructions for treating a viral infection.

In a further aspect, the viral infection is human immunodeficiency virus (HIV), human papillomavirus (HPV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, yellow fever virus, tick-bome encephalitis virus, Chikungunya virus (CHIKV), Venezuelan equine encephalitis (VEEV), Eastern equine encephalitis (EEEV), Western equine encephalitis (WEEV), dengue (DENV), influenza, West Nile virus (WNV), zika (ZIKV), Middle East Respiratory Syndromes (MERS), Severe Acute Respiratory Syndrome (SARS), coronavirus disease 2019 (COVID-19), or influenza virus. In a still further aspect, the viral infection is influenza virus.

In a further aspect, the antiviral agent is selected from selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscamet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, MK-4482 (EIDD-2801), penciclovir, raltegravir, ribavirin, rimantadine hydrochloride, rilpivirine (TMC-278), remdesivir, saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, In a further aspect, the immunity booster is selected from vitamin D, elderberry, Echinacea, a probiotic, vitamin C, vitamin B, green tea, turmeric, zinc, ashwagandha, a prebiotic, and a synbiotic.

In a further aspect, the compound and the antiviral agent are co-formulated. In a further aspect, the compound and the antiviral agent are co-packaged.

In a further aspect, the compound and the immunity booster are co-formulated.

In a further aspect, the compound and the immunity booster are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred aspects but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The aspects described herein above are further intended to explain best modes known by applicant and to enable others skilled in the art to utilize the disclosure in such, or other, aspects and with the various modifications required by the particular applications or uses thereof. Accordingly, the description is not intended to limit the invention to the form disclosed herein. Also, it is intended to the appended claims be construed to include alternative aspects.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### G. Examples

Recently, progress was reported in the development of new small molecules that specifically target the active site of the PA influenza endonuclease, and details disclosed regarding the molecular interactions of these new inhibitors with an influenza endonuclease construct. The overarching approach, as detailed herein, has shifted to proactively develop lead inhibitors that are less likely to rapidly develop clinical resistance by optimizing inhibitors that retain activity against induced resistant mutants. Here, the details behind the discovery of new potent 9-hydroxy-6-substituted-3,4-dihydro-2H-pyrazino[1,2-c]pyrimidine-1,8-dione inhibitors of influenza virus endonuclease having improved physiochemical properties are disclosed. Without wishing to be bound by theory, these results further an understanding of nuclease protein targets, and potentially serve as starting points for a new therapeutic approach to the treatment of influenza.

The essential endonuclease domain within the PA subunit is a particularly attractive drug target. It has no eukaryotic homolog, so the potential for toxicity due to off-target effects is reduced for small molecules that target its active site. The influenza virus RdRp is a heterotrimer that includes the polymerase catalytic subunit (PB1), the “cap-binding” subunit (PB2), and the endonuclease-containing (PA) subunit, which is conserved across genotypes, and is essential for viral replication. The “cap-binding” and endonuclease functionalities of RdRp work in concert to perform the essential “cap snatching” of host mRNAs to generate primers for viral transcription. Over the last ten years, the understanding of influenza viral RdRp has dramatically expanded through the elucidation of the high-resolution architecture of influenza endonuclease and the publication of the RdRp heterotrimer structure.

Using the structure of the domain determined in isolation and in the context of the trimeric complex a number of groups have successfully used structure-assisted approaches to develop potent inhibitors. More recently, inventors have reported the development of potent inhibitors of wild type and mutant influenza virus endonuclease with the general structure 1. High-resolution co-crystal structures of analogs of 1 with N-terminal constructs of PA (PAN) have been previously reported, which led to an improved understanding of the molecular determinants of inhibition for both the wild type and E119D mutant inhibitor resistant influenza endonucleases. This, in turn, led to the discovery of several lead chemotypes and pharmacophores that strongly interact with conserved residues in the active site of PA.

In the course of this work, unique binding conformations of 1 analogs bound to endonuclease constructs was observed, and several new types of tight-binding interactions discovered. The primary factor that limits the advancement of leads based on the 1 substructure are the physiochemical properties of this class of compounds. In order to improve the physiochemical properties of the two-metal binding core, conformationally constrained core structure 2 that reduces rotatable bonds and hydrogen bond donors in the core structure, both of which are important factors in determining cell wall permeability, was designed. In the following the synthesis, PAN binding affinity and influenza plaque inhibition of this novel class of anti-influenza agents are described. Structure 1 below is representative of a recently published influenza virus endonuclease inhibitor class. Structure 2 is one example of a new conformationally constrained inhibitor as further disclosed herein.

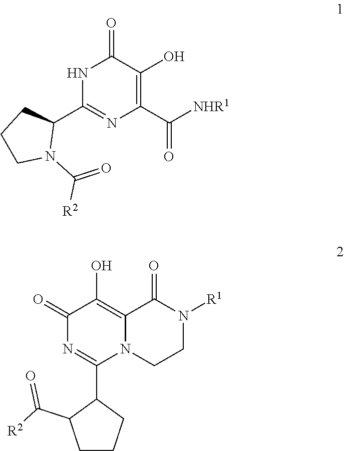

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimentals

Compounds disclosed herein demonstrate antiviral activity as shown in the tables below. Shown below are IC₅₀ and Ki values of the disclosed compounds.

### a. General Synthesis of Aryl Sulfones

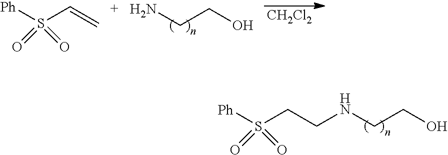

Combined phenyl vinyl sulfone (1 eq) and the amino alcohol (1 eq) in dichloromethane (0.15 M) and stirred at room temperature overnight. Concentrated and used directly in the next step.

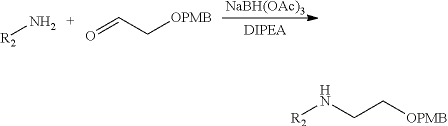

### i. DJB-16644-50

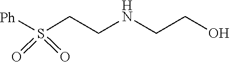 [SMILES: O=S(=O)(CCNCCO)c1ccccc1]

¹H NMR (399 MHz, Chloroform-d) δ 8.00-7.86 (m, 2H), 7.72-7.63 (m, 1H), 7.63-7.51 (m, 2H), 3.64-3.57 (m, 2H), 3.30 (t, J=6.4 Hz, 2H), 3.06 (t, J=6.4 Hz, 2H), 2.79-2.70 (m, 2H). LCMS (ESI): m/z=230 [M+H]⁺.

### ii. DJB-16644-43

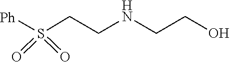 [SMILES: O=S(=O)(CCNCCO)c1ccccc1]

¹H NMR (399 MHz, Chloroform-d) δ 7.94-7.87 (m, 2H), 7.71-7.65 (m, 1H), 7.61-7.54 (m, 2H), 3.79-3.74 (m, 2H), 3.27 (t, J=6.4 Hz, 2H), 3.05 (t, J=6.4 Hz, 2H), 2.87-2.79 (m, 2H), 1.68 (dt, J=10.9, 5.6 Hz, 2H). LCMS (ESI): m/z=244 [M+H]⁺.

### iii. JP-16547-11-1

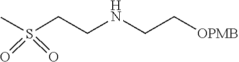

¹H NMR (399 MHz, Chloroform-d) δ 7.25 (d, J=8.6 Hz, 2H), 6.88 (d, J=8.7 Hz, 2H), 4.45 (s, 2H), 3.81 (s, 3H), 3.56 (t, J=5.1 Hz, 2H), 3.22-3.12 (m, 4H), 2.99 (s, 3H), 2.84 (t, J=5.1 Hz, 2H). LCMS (ESI): m/z=288 [M+H]⁺.

### iv. CL-16543-12

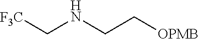

¹H NMR (399 MHz, Chloroform-d) δ 7.33-7.21 (m, 2H), 6.93-6.78 (m, 2H), 4.44 (s, 2H), 3.79 (d, J=2.8 Hz, 4H), 3.55 (t, J=5.0 Hz, 2H), 3.18 (q, J=9.5 Hz, 2H), 2.90 (t, J=5.0 Hz, 2H), 2.05 (br s, 1H). LCMS (ESI): m/z=264 [M+H]⁺.

### v. CL-16617-72

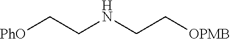

¹H NMR (399 MHz, Chloroform-d) δ 7.28-7.22 (m, 4H), 7.00-6.77 (m, 5H), 4.45 (s, 2H), 4.05 (t, J=4.7 Hz, 2H), 3.78 (s, 3H), 3.58 (t, J=4.8 Hz, 2H), 3.01 (t, J=4. Hz, 2H), 2.87 (t, J=4.7 Hz, 2H). LCMS (ESI): m/z=302 [M+H]⁺.

### vi. CL-16617-64

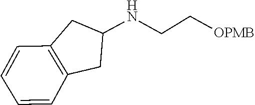

¹H NMR (399 MHz, Chloroform-d) δ 7.25 (d, J=8.5 Hz, 2H), 7.20-7.07 (m, 4H), 6.86 (d, J=8.5 Hz, 2H), 4.44 (s, 2H), 3.78 (s, 3H), 3.62-3.55 (m, 3H), 3.13 (dd, J=15.5, 7.2 Hz, 2H), 2.85 (dd, J=5.6, 4.8 Hz, 2H), 2.74 (dd, J=15.5, 6.7 Hz, 2H). LCMS (ESI): m/z=298 [M+H]⁺.

### b. General Synthesis of 9-hydroxy-6-(pyrrolidin-2-yl)-3,4-dihydro-2H-pyrazino[1,2-c]pyrimidine-1,8-dione Derivatives

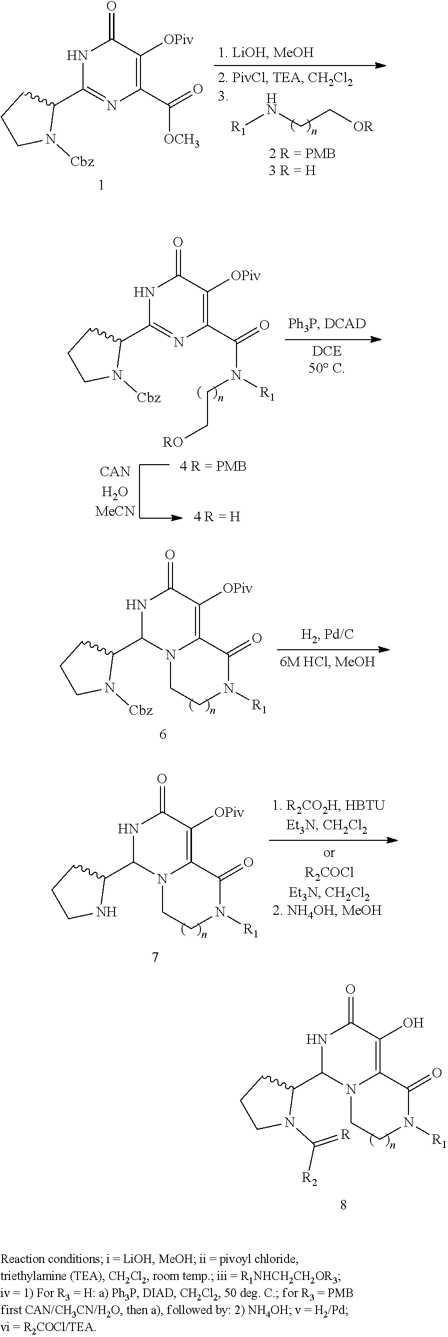

Briefly, combined 1 (1 eq) and pivaloyl chloride (2 eq) in CH₂Cl₂ (0.07 M), then added TEA (2.5 eq) and stirred at room temperature overnight. Then, added substituted amine (R₁NHCH₂CH₂OH) directly in minimal CH₂Cl₂ and stirred for 5 hours at room temperature. Concentrated and put directly on a reverse phase column to purify (MeOH/H₂O/0.1% formic acid).

Added PPh₃ (1.5 eq) and DIAD (1.4 eq) to a solution of 5 in 1,2-dichloroethane (0.06 M). Then, stirred and heated to 50° C. overnight. When the reaction was complete, concentrated and ran a column directly (10-50% acetone in CH₂Cl₂).

To a solution of compound 4 in acetonitrile (0.013 M) and water (0.038 M) at 0° C. was added CAN (4 eq). The reaction was warmed to room temperature over 1 hour. Then the reaction mixture was concentrated and put directly on a reverse phase column (MeOH/H₂O/0.1% formic acid).

To a solution of compound 6 (1 eq.) in methanol (0.33 M) was added 10% wetted Pd/C (1 eq.) and 6M HCl (1 eq.). The reaction mixture was placed under a balloon of H₂ and stirred at room temperature for 2 h. The reaction mixture was then diluted with methanol and ethyl acetate and filtered through a pad of celite. The filtrate was concentrated in vacuo to obtain the desired product 7.

To a mixture of carboxylic acid (1 eq.) in dry DMF at room temperature was added triethylamine (2 eq.), followed by HBTU (1.1 eq.) and amine (1.1 eq), OR to amine 7 (1 eq.) in dry DCM (1 mL) was added acid chloride (1 eq.) followed by triethylamine (3 eq.).

Once the reaction was complete, quenched with aqueous NH₄Cl and diluted with ethyl acetate. The organic layer was washed with water and brine, dried over MgSO₄, filtered, and concentrated. The crude product was used directly in the next step.

To the crude product in MeOH (0.5 M) was added NH₄OH dropwise. The reaction stirred at room temperature overnight. When the reaction was complete, the solution was concentrated. The crude product was purified by reverse phase chromatography (MeOH/water+25 mM NH₄OAc pH 7.4).

### i. (S)-2-(1-((benzyloxy)carbonyl)pyrrolidin-2-yl)-5-hydroxy-6-oxo-1,6-dihydropyrimidine-4-carboxylic acid

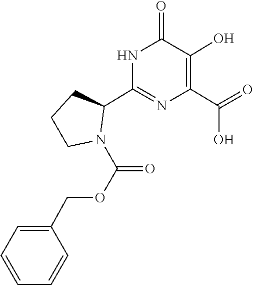 [SMILES: O=C(O)c1nc([C@@H]2CCCN2C(=O)OCc2ccccc2)[nH]c(=O)c1O]

¹H NMR (399 MHz, DMSO-d₆) δ 7.38-7.23 (m, 3H), 7.22-7.12 (m, 1H), 7.07-6.94 (m, 1H), 5.12-4.94 (m, 2H), 4.80 (d, J=12.9 Hz, 1H), 4.67-4.55 (m, 1H), 3.66-3.52 (m, 1H), 3.47-3.32 (m, 1H), 2.28-2.13 (m, 1H), 1.98-1.70 (m, 3H). LCMS (ESI): m/z=360 [M+H]⁺.

### ii. JP-16547-42-1

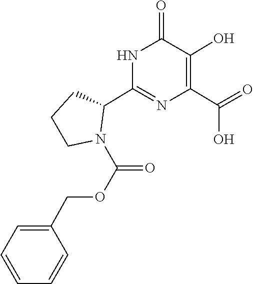 [SMILES: O=C(O)c1nc([C@H]2CCCN2C(=O)OCc2ccccc2)[nH]c(=O)c1O]

¹H NMR (399 MHz, DMSO-d₆) δ 7.36-7.20 (m, 3H), 7.20-7.08 (m, 1H), 7.01-6.94 (m, 1H), 5.09-4.96 (m, 2H), 4.78 (d, J=12.9 Hz, 1H), 4.64-4.52 (m, 1H), 3.63-3.46 (m, 1H), 3.43-3.26 (m, 1H), 2.25-2.10 (m, 1H), 1.96-1.68 (m, 3H). LCMS (ESI): m/z=360 [M+H]⁺.

### iii. DJB-16644-36A

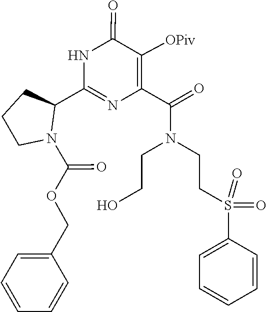

¹H NMR (400 MHz, Chloroform-d) δ 7.97-7.79 (m, 2H), 7.74-7.63 (m, 1H), 7.61-7.48 (m, 2H), 7.45-7.28 (m, 5H), 5.25-5.02 (m, 2H), 4.79-4.68 (m, 1H), 3.87-3.35 (m, 10H), 2.33-2.14 (m, 1H), 2.06-1.82 (m, 3H), 1.28 (d, J=10.4 Hz, 9H). LCMS (ESI): m/z=655 [M+H]⁺.

### iv. DJB-16644-73B

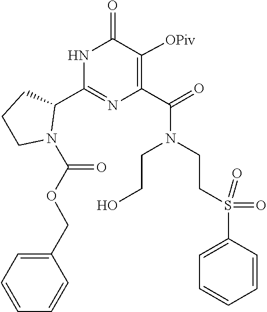

¹H NMR (400 MHz, Chloroform-d) δ 7.96-7.83 (m, 2H), 7.73-7.63 (m, 1H), 7.63-7.51 (m, 2H), 7.42-7.28 (m, 5H), 5.26-5.04 (m, 2H), 4.83-4.69 (m, 1H), 3.88-3.35 (m, 10H), 2.29-2.13 (m, 1H), 2.07-1.84 (m, 3H), 1.28 (d, J=10.9 Hz, 9H). LCMS (ESI): m/z=655 [M+H]⁺.

### v. DJB-16644-37

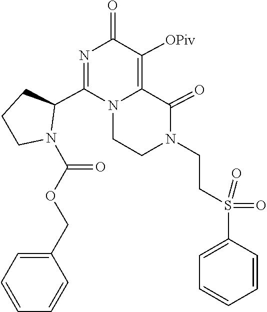

¹H NMR (400 MHz, Chloroform-d) δ 7.97-7.83 (m, 2H), 7.73-7.64 (m, 1H), 7.59 (t, J=7.8 Hz, 2H), 7.39-7.27 (m, 5H), 5.11 (d, J=12.4 Hz, 1H), 4.99 (d, J=12.4 Hz, 1H), 4.94-4.81 (m, 1H), 4.81-4.70 (m, 1H), 4.23-4.13 (m, 1H), 4.13-3.17 (m, 8H), 2.39-2.12 (m, 3H), 2.00-1.83 (m, 1H), 1.36 (d, J=10.7 Hz, 9H). LCMS (ESI): m/z=637 [M+H]⁺.

### vi. DJB-16644-75B

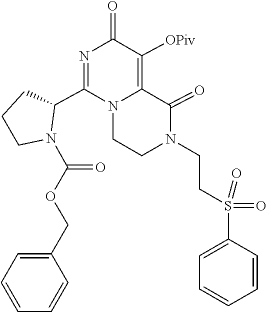

¹H NMR (400 MHz, Chloroform-d) δ 7.96-7.80 (m, 2H), 7.72-7.61 (m, 1H), 7.61-7.50 (m, 2H), 7.42-7.26 (m, 5H), 5.10 (d, J=12.3 Hz, 1H), 4.98 (d, J=12.4 Hz, 1H), 4.95-4.82 (m, 1H), 4.80-4.70 (m, 1H), 4.26-4.16 (m, 1H), 4.11-3.15 (m, 10H), 2.39-2.19 (m, 3H), 1.98-1.85 (m, 1H), 1.35 (d, J=10.4 Hz, 9H). LCMS (ESI): m/z=637 [M+H]⁺.

### vii. SRI-030287 (DJB-16644-39A)

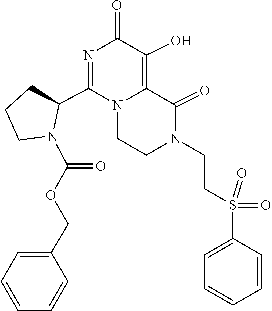 [SMILES: O=C1c2c(O)c(=O)nc([C@@H]3CCCN3C(=O)OCc3ccccc3)n2CCN1CCS(=O)(=O)c1ccccc1]

¹H NMR (400 MHz, Chloroform-d) δ 7.91 (d, J=7.0 Hz, 2H), 7.68 (t, J=7.4 Hz, 1H), 7.64-7.55 (m, 2H), 7.39-7.27 (m, 5H), 5.11 (d, J=12.3 Hz, 1H), 5.00 (d, J=12.5 Hz, 1H), 4.94-4.88 (m, 1H), 4.80-4.69 (m, 1H), 4.28-4.15 (m, 1H), 4.15-3.26 (m, 8H), 2.38-2.06 (m, 3H), 1.98-1.81 (m, 1H). LCMS (ESI): m/z=553 [M+H]⁺.

### viii. SRI-030406 (DJB-16644-79A)

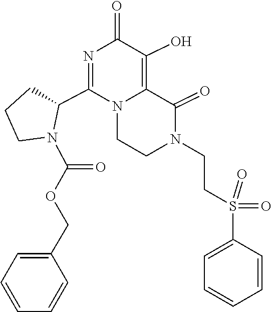 [SMILES: O=C1c2c(O)c(=O)nc([C@H]3CCCN3C(=O)OCc3ccccc3)n2CCN1CCS(=O)(=O)c1ccccc1]

¹H NMR (400 MHz, Methanol-d₄) δ 8.02-7.88 (m, 2H), 7.77-7.55 (m, 3H), 7.37-7.26 (m, 2H), 7.26-7.17 (m, 2H), 7.14-7.04 (m, 1H), 5.14 (d, J=11.6 Hz, 1H), 5.09-4.98 (m, 1H), 4.74 (d, J=11.7 Hz, 1H), 4.65-4.42 (m, 2H), 4.18 (dd, J=36.7, 18.7 Hz, 2H), 3.98-3.35 (m, 6H), 2.45-2.25 (m, 1H), 2.22-2.07 (m, 1H), 2.05-1.81 (m, 2H). LCMS (ESI): m/z=553 [M+H]⁺.

### ix. SRI-030407 (DJB-16644-76A)

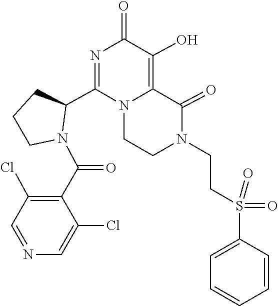 [SMILES: O=C1c2c(O)c(=O)nc([C@@H]3CCCN3C(=O)c3c(Cl)cncc3Cl)n2CCN1CCS(=O)(=O)c1ccccc1]

¹H NMR (400 MHz, Methanol-d₄) δ 8.62 (d, J=2.8 Hz, 2H), 7.96 (dt, J=7.1, 1.4 Hz, 2H), 7.76-7.68 (m, 1H), 7.67-7.59 (m, 2H), 5.38 (dd, J=8.1, 5.2 Hz, 1H), 4.72-4.60 (m, 1H), 4.44-4.33 (m, 1H), 4.02-3.91 (m, 2H), 3.91-3.81 (m, 2H), 3.71-3.57 (m, 4H), 2.53-2.41 (m, 1H), 2.39-2.15 (m, 2H), 2.08-1.97 (m, 1H). LCMS (ESI): m/z=592 [M+H]⁺.

### x. SRI-030330 (DJB-16644-71A-F9)

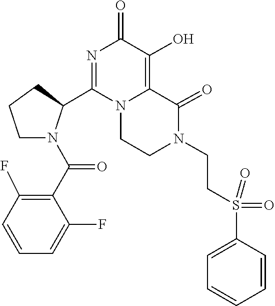 [SMILES: O=C1c2c(O)c(=O)nc([C@@H]3CCCN3C(=O)c3c(F)cccc3F)n2CCN1CCS(=O)(=O)c1ccccc1]

¹H NMR (400 MHz, Chloroform-d) δ 7.95-7.88 (m, 2H), 7.72-7.65 (m, 1H), 7.59 (dd, J=8.3, 7.0 Hz, 2H), 7.36 (tt, J=8.3, 6.4 Hz, 1H), 6.94 (t, J=8.2 Hz, 2H), 5.26-5.17 (m, 1H), 5.08-4.96 (m, 1H), 4.33-4.21 (m, 1H), 4.16-4.00 (m, 2H), 3.99-3.84 (m, 2H), 3.80-3.69 (m, 1H), 3.63-3.52 (m, 1H), 3.49-3.32 (m, 2H), 2.47-2.29 (m, 3H), 1.99-1.89 (m, 1H). LCMS (ESI): m/z=559 [M+H]⁺.

### xi. SRI-030405 (DJB-16644-78A)

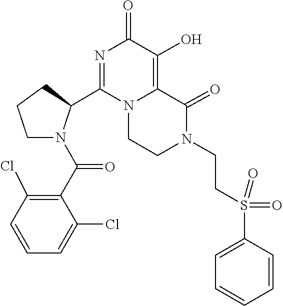 [SMILES: O=C1c2c(O)c(=O)nc([C@@H]3CCCN3C(=O)c3c(Cl)cccc3Cl)n2CCN1CCS(=O)(=O)c1ccccc1]

¹H NMR (400 MHz, Methanol-d₄) δ 7.96 (d, J=7.5 Hz, 2H), 7.75-7.66 (m, 1H), 7.62 (t, J=7.4 Hz, 2H), 7.49-7.38 (m, 3H), 5.34 (dd, J=8.2, 5.3 Hz, 1H), 4.74-4.63 (m, 1H), 4.45-4.33 (m, 1H), 4.04-3.80 (m, 4H), 3.67 (t, J=6.4 Hz, 2H), 3.58 (dt, J=10.3, 7.3 Hz, 1H), 3.40-3.32 (m, 1H), 2.53-2.41 (m, 1H), 2.37-2.29 (m, 1H), 2.27-2.18 (m, 1H), 2.06-1.91 (m, 1H). LCMS (ESI): m/z=591 [M+H]⁺.

### xii. SRI-030526 (DJB-16644-85A)

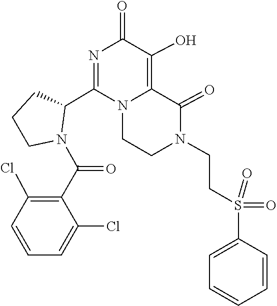 [SMILES: O=C1c2c(O)c(=O)nc([C@H]3CCCN3C(=O)c3c(Cl)cccc3Cl)n2CCN1CCS(=O)(=O)c1ccccc1]

¹H NMR (400 MHz, Methanol-d₄) δ 8.03-7.88 (m, 2H), 7.73-7.68 (m, 1H), 7.66-7.61 (m, 2H), 7.49-7.38 (m, 3H), 5.35 (dd, J=8.1, 5.3 Hz, 1H), 4.75-4.66 (m, 1H), 4.44-4.33 (m, 1H), 4.02-3.92 (m, 2H), 3.87 (t, J=5.6 Hz, 2H), 3.68 (t, J=6.4 Hz, 2H), 3.64-3.54 (m, 1H), 3.41-3.33 (m, 1H), 2.51-2.40 (m, 1H), 2.40-2.28 (m, 1H), 2.28-2.20 (m, 1H), 2.03-1.95 (m, 1H). LCMS (ESI): m/z=591 [M+H]⁺.

### xiii. SRI-030662 (CL-16617-93)

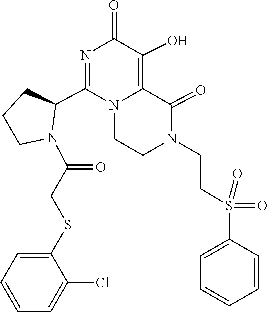 [SMILES: O=C1c2c(O)c(=O)nc([C@@H]3CCCN3C(=O)CSc3ccccc3Cl)n2CCN1CCS(=O)(=O)c1ccccc1]

¹H NMR (399 MHz, Methanol-d₄) δ 7.99-7.87 (m, 2H), 7.70-7.55 (m, 3H), 7.37-7.17 (m, 3H), 7.12 (ddd, J=7.9, 7.3, 1.7 Hz, 1H), 5.18-5.08 (m, 1H), 4.52-4.40 (m, 1H), 4.26-4.14 (m, 1H), 4.00-3.86 (m, 4H), 3.82-3.73 (m, 4H), 3.64 (t, J=6.4 Hz, 2H), 2.37-2.21 (m, 2H), 2.11-1.96 (m, 2H). LCMS (ESI): m/z=603 [M+H]⁺.

### xiv. SRI-030663 (CL-16617-94)

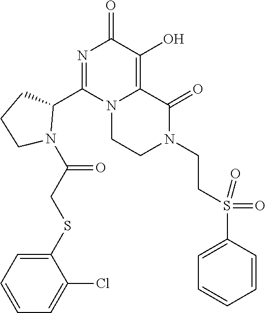 [SMILES: O=C1c2c(O)c(=O)nc([C@H]3CCCN3C(=O)CSc3ccccc3Cl)n2CCN1CCS(=O)(=O)c1ccccc1]

¹H NMR (399 MHz, Methanol-d₄) δ 8.00-7.89 (m, 2H), 7.68-7.52 (m, 3H), 7.38-7.17 (m, 3H), 7.16-7.03 (m, 1H), 5.18-5.06 (m, 1H), 4.52-4.40 (m, 1H), 4.28-4.12 (m, 1H), 4.00-3.84 (m, 4H), 3.83-3.74 (m, 4H), 3.64 (t, J=6.4 Hz, 2H), 2.40-2.19 (m, 2H), 2.11-1.96 (m, 2H). LCMS (ESI): m/z=603 [M+H]⁺.

### xv. DJB-16644-42A

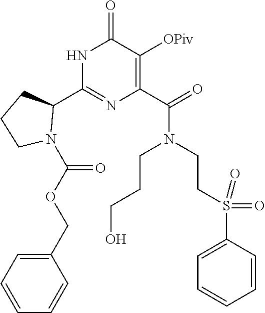

¹H NMR (400 MHz, Chloroform-d) δ 7.89 (dd, J=34.6, 7.1 Hz, 2H), 7.74-7.50 (m, 3H), 7.45-7.28 (m, 5H), 5.27-5.01 (m, 2H), 4.82-4.68 (m, 1H), 3.83-3.31 (m, 10H), 2.06-1.88 (m, 2H), 1.83-1.47 (m, 4H), 1.29 (d, J=14.2 Hz, 9H). LCMS (ESI): m/z=669 [M+H]⁺.

### xvi. DJB-16644-44A

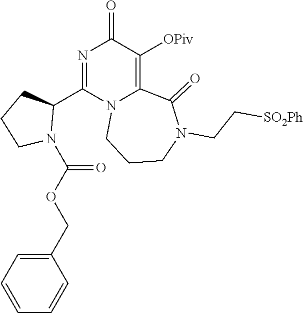

¹H NMR (400 MHz, Chloroform-d) δ 7.99-7.86 (m, 2H), 7.75-7.46 (m, 3H), 7.40-7.29 (m, 5H), 5.12 (d, J=12.4 Hz, 1H), 4.99 (d, J=12.4 Hz, 1H), 4.96-4.83 (m, 1H), 4.05-3.09 (m, 10H), 2.43-1.81 (m, 6H), 1.29 (d, J=8.3 Hz, 9H). LCMS (ESI): m/z=651 [M+H]⁺.

### xvii. SRI-030291 (DJB-16644-54)

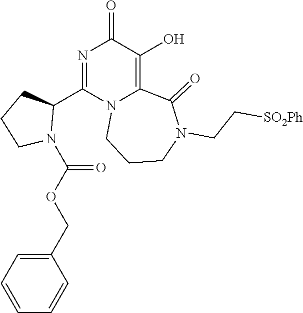

¹H NMR (400 MHz, Chloroform-d) δ 7.96-7.87 (m, 2H), 7.72-7.63 (m, 1H), 7.62-7.51 (m, 2H), 7.37-7.27 (m, 5H), 5.09 (d, J=12.2 Hz, 1H), 4.99 (d, J=12.4 Hz, 1H), 4.86 (dd, J=7.5, 4.6 Hz, 1H), 4.07-3.28 (m, 10H), 2.42-1.78 (m, 6H). LCMS (ESI): m/z=567 [M+H]⁺.

### xviii. DJB-16644-13A

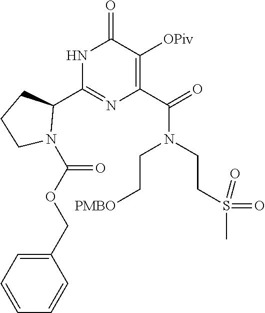

¹H NMR (399 MHz, DMSO-d₆) δ 7.37-7.13 (m, 6H), 7.10-7.01 (m, 1H), 6.90-6.79 (m, 2H), 5.12-4.82 (m, 4H), 4.70-4.53 (m, 1H), 3.79-3.72 (m, 2H), 3.72-3.66 (m, 3H), 3.64-3.24 (m, 8H), 2.94 (s, 3H), 2.33-2.16 (m, 1H), 1.97-1.73 (m, 3H), 1.17 (t, J=6.9 Hz, 9H). LCMS (ESI): m/z=713 [M+H]⁺.

### xix. DJB-16586-93A

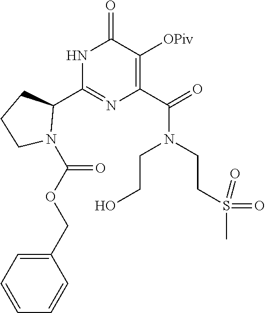

¹H NMR (400 MHz, DMSO-d₆) δ 7.37-7.31 (m, 2H), 7.31-7.15 (m, 2H), 7.11-7.03 (m, 1H), 5.12-4.87 (m, 2H), 4.66-4.56 (m, 1H), 3.74 (dd, J=9.5, 5.8 Hz, 1H), 3.63-3.15 (m, 9H), 2.99 (d, J=1.2 Hz, 3H), 2.25 (ddd, J=17.6, 13.1, 8.4 Hz, 1H), 1.98-1.73 (m, 3H), 1.19 (d, J=5.7 Hz, 9H). LCMS (ESI): m/z=593 [M+H]⁺.

### xx. DJB-16586-91A

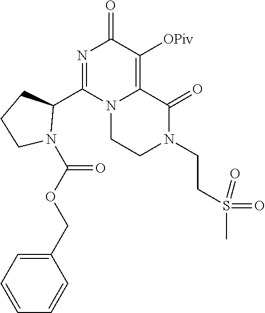

¹H NMR (400 MHz, Chloroform-d) δ 7.40-7.28 (m, 5H), 5.11 (d, J=12.3 Hz, 1H), 4.99 (d, J=12.4 Hz, 1H), 4.91-4.79 (m, 2H), 4.21-3.19 (m, 9H), 2.99 (s, 3H), 2.42-2.07 (m, 3H), 2.00-1.81 (m, 1H), 1.39 (d, J=10.6 Hz, 9H). LCMS (ESI): m/z=575 [M+H]⁺.

### xxi. CL-16617-41-1

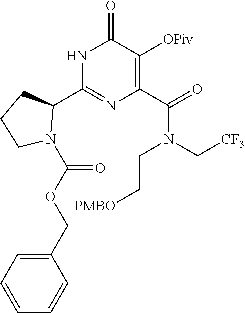

¹H NMR (399 MHz, Chloroform-d) δ 7.43-7.29 (m, 5H), 7.19 (d, J=8.6 Hz, 2H), 6.87 (d, J=8.7 Hz, 2H), 5.25-5.07 (m, 4H), 4.87-4.73 (m, 1H), 4.49-4.27 (m, 4H), 3.80 (s, 3H), 3.71-3.31 (m, 4H), 2.06-1.85 (m, 3H), 1.71-1.50 (m, 1H), 1.30 (d, J=2.0 Hz, 9H). LCMS (ESI): m/z=689 [M+H]⁺.

### xxii. CL-16617-42-1

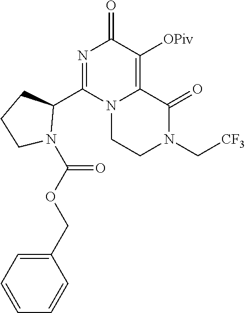

¹H NMR (399 MHz, Chloroform-d) δ 7.42-7.29 (m, 5H), 5.14 (q, J=12.2 Hz, 2H), 4.77 (t, J=6.3 Hz, 1H), 4.32-3.96 (m, 2H), 3.78-3.43 (m, 6H), 2.30-2.17 (m, 1H), 2.12-1.85 (m, 3H), 1.31 (d, J=7.3 Hz, 9H). LCMS (ESI): m/z=569 [M+H]⁺.

### xxiii. CL-16617-42-1

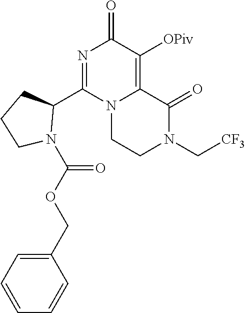

¹H NMR (399 MHz, Chloroform-d) δ 7.41-7.29 (m, 5H), 5.11 (d, J=12.4 Hz, 1H), 4.99 (d, J=12.4 Hz, 1H), 4.89-4.80 (m, 1H), 4.12-3.63 (m, 5H), 3.63-3.49 (m, 1H), 2.42-2.18 (m, 3H), 2.00-1.82 (m, 1H), 1.38 (d, J=11.1 Hz, 9H). LCMS (ESI): m/z=551 [M+H]⁺.

### xxiv. CL-16617-73

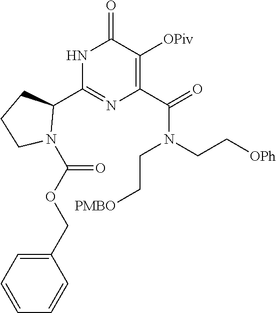

¹H NMR (399 MHz, Chloroform-d) δ 7.33 (s, 3H), 7.24-7.11 (m, 6H), 6.96-6.86 (m, 1H), 6.87-6.79 (m, 3H), 6.75 (d, J=8.1 Hz, 1H), 5.24-4.99 (m, 2H), 4.77 (s, 1H), 4.39 (d, J=16.0 Hz, 2H), 4.16 (t, J=5.4 Hz, 1H), 4.09-4.02 (m, 1H), 3.94-3.88 (m, 1H), 3.83-3.73 (m, 3H), 3.68 (q, J=6.4, 5.5 Hz, 2H), 3.65-3.36 (m, 4H), 2.58-2.47 (m, 1H), 2.05-1.78 (m, 4H), 1.25 (s, 5H), 1.16 (s, 4H). LCMS (ESI): m/z=727 [M+H]⁺.

### xxv. CL-16617-74

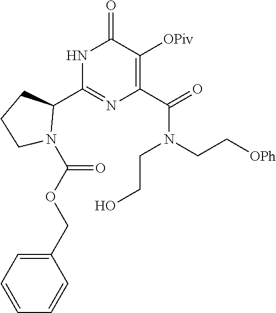

¹H NMR (399 MHz, Chloroform-d) δ 7.38-7.19 (m, 6H), 7.15 (s, 1H), 6.94 (td, J=7.4, 3.5 Hz, 1H), 6.90-6.79 (m, 2H), 5.23-5.03 (m, 2H), 4.76 (s, 1H), 4.22 (t, J=5.3 Hz, 1H), 3.84 (d, J=5.4 Hz, 2H), 3.82-3.72 (m, 2H), 3.59 (d, J=41.9 Hz, 4H), 3.32-3.24 (m, 1H), 2.25 (s, 2H), 2.12-1.79 (m, 3H), 1.28 (s, 3H), 1.17 (s, 6H). LCMS (ESI): m/z=607 [M+H]⁺.

### xxvi. CL-16617-75

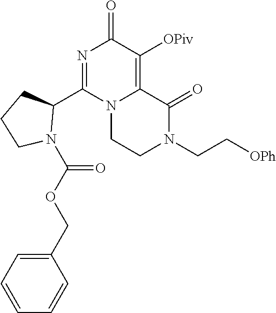

¹H NMR (399 MHz, Chloroform-d) δ 7.36-2.27 (m, 5H), 7.22-7.05 (m, 2H), 6.97 (dd, J=8.0, 6.7 Hz, 1H), 6.88-6.77 (m, 2H), 5.14-4.95 (m, 2H), 4.95-4.70 (m, 2H), 4.29-4.07 (m, 2H), 4.05-3.92 (m, 2H), 3.88-3.61 (m, 4H), 3.54 (dt, J=10.3, 7.2 Hz, 1H), 2.34 (dq, J=13.8, 7.0 Hz, 1H), 2.29-2.11 (m, 2H), 1.88 (dt, J=13.0, 6.6 Hz, 1H), 1.38 (d, J=11.9 Hz, 9H). LCMS (ESI): m/z=589 [M+H]⁺.

### xxvii. SRI-030395 (CL-16617-76)

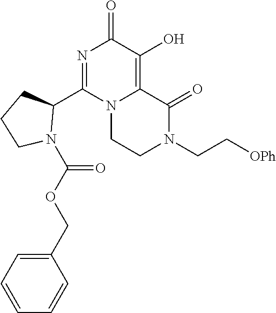 [SMILES: O=C1c2c(O)c(=O)nc([C@@H]3CCCN3C(=O)OCc3ccccc3)n2CCN1CCOc1ccccc1]

¹H NMR (399 MHz, Methanol-d₄) δ 7.39-7.21 (m, 4H), 7.14-7.03 (m, 2H), 7.01-6.90 (m, 4H), 5.15-5.04 (m, 2H), 5.00 (dd, J=8.0, 5.3 Hz, 1H), 4.66 (d, J=11.5 Hz, 1H), 4.62-4.49 (m, 1H), 4.33-4.10 (m, 3H), 4.06-3.89 (m, 2H), 3.85-3.65 (m, 2H), 3.59 (ddt, J=10.2, 7.3, 3.5 Hz, 1H), 3.48 (ddd, J=13.6, 7.4, 3.7 Hz, 1H), 2.35 (dt, J=12.4, 7.4 Hz, 1H), 2.24-2.09 (m, 1H), 2.10-1.86 (m, 2H). LCMS (ESI): m/z=505 [M+H]⁺.

### xxviii. CL-16617-66

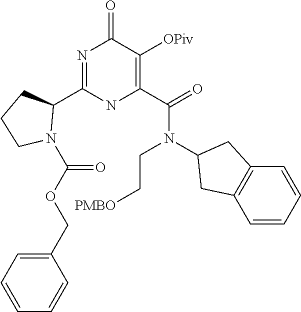

¹H NMR (399 MHz, Chloroform-d) δ 7.35-7.27 (m, 4H), 7.20 (d, J=8.5 Hz, 2H), 7.16-7.08 (m, 5H), 6.84 (d, J=8.5 Hz, 2H), 5.23-5.03 (m, 2H), 4.85-4.80 (m, 1H), 4.58-5.51 (m, 1H), 4.39 (s, 2H), 3.77 (s, 3H), 3.66 (t, J=6.2 Hz, 2H), 3.52 (t, J=6.2 Hz, 2H), 3.46-3.41 (m, 2H), 3.14-2.96 (m, 4H), 2.71-2.62 (m, 1H), 2.12-1.86 (m, 4H), 1.31 (d, J=9.1 Hz, 9H). LCMS (ESI): m/z=723 [M+H]⁺.

### xxix. CL-16617-67

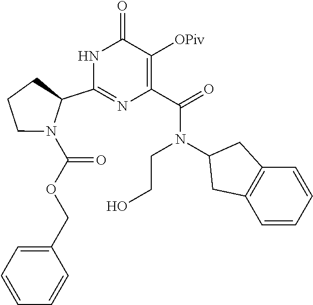

¹H NMR (399 MHz, Chloroform-d) δ 7.35-7.27 (m, 4H), 7.15-7.10 (m, 5H), 5.22-5.01 (m, 2H), 4.85-4.78 (m, 1H), 4.64-4.55 (m, 1H), 3.77 (t, J=4.9 Hz, 2H), 3.54 (t, J=5.1 Hz, 2H), 3.29-3.06 (m, 4H), 2.97 (dd, J=16.4, 7.1 Hz, 2H), 2.68-2.58 (m, 1H), 2.32-2.14 (m, 1H), 2.11-1.85 (m, 4H), 1.33 (d, J=9.1 Hz, 9H). LCMS (ESI): m/z=603 [M+H]⁺.

### xxx. CL-16617-68

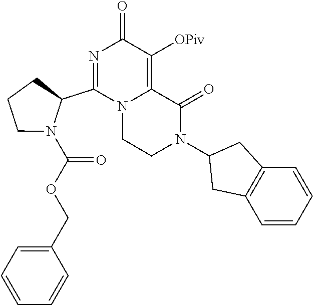

¹H NMR (399 MHz, Chloroform-d) δ 7.37-7.25 (m, 4H), 7.22-7.14 (m, 5H), 5.55-5.36 (m, 1H), 5.15-4.91 (m, 2H), 4.85-4.65 (m, 2H), 3.93-3.70 (m, 2H), 3.68-3.49 (m, 2H), 3.38-3.24 (m, 3H), 3.04-2.78 (m, 2H), 2.40-2.31 (m, 1H), 2.28-2.08 (m, 2H), 1.91-1.82 (m, 1H), 1.39 (d, J=10.3 Hz, 9H). LCMS (ESI): m/z=585 [M+H]⁺.

### xxxi. CL-16617-10-1

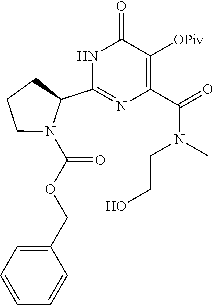

¹H NMR (399 MHz, Chloroform-d) δ 7.40-7.25 (m, 4H), 7.20-7.07 (m, 1H), 5.30-5.03 (m, 2H), 4.86-4.63 (m, 1H), 3.89-3.28 (m, 6H), 3.04 (s, 3H), 2.22 (d, J=7.1 Hz, 1H), 2.12-1.82 (m, 3H), 1.35-1.25 (m, 9H). LCMS (ESI): m/z=501 [M+H]⁺.

### xxxii. CL-16617-07-1

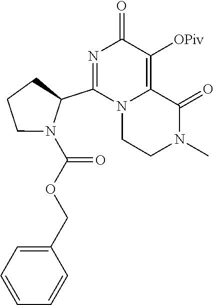

¹H NMR (399 MHz, Chloroform-d) δ 7.40-7.29 (m, 5H), 5.11 (d, J=12.3 Hz, 1H), 4.99 (d, J=12.6 Hz, 1H), 4.96-4.79 (m, 1H), 4.10-3.68 (m, 4H), 3.63-3.43 (m, 2H), 3.12 (s, 3H), 2.47-1.80 (m, 4H), 1.46-1.34 (m, 9H). LCMS (ESI): m/z=483 [M+H]⁺.

### xxxiii. CL-16617-19-1

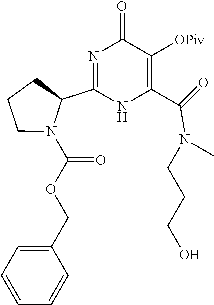

¹H NMR (399 MHz, Chloroform-d) δ 7.44-7.28 (m, 5H), 5.13 (dd, J=30.4, 15.6 Hz, 2H), 4.87-4.68 (m, 1H), 3.52 (d, J=51.4 Hz, 6H), 2.90 (s, 3H), 2.45-1.50 (m, 6H), 1.31 (d, J=12.9 Hz, 9H). LCMS (ESI): m/z=515 [M+H]⁺.

### xxxiv. CL-16617-20-2

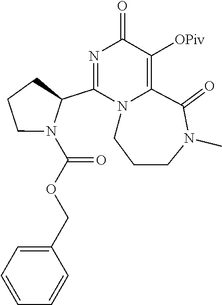

¹H NMR (399 MHz, Methanol-d₄) δ 7.40-7.23 (m, 4H), 7.18-7.00 (m, 1H), 5.26-4.92 (m, 3H), 3.93-3.36 (m, 6H), 3.04-2.91 (m, 3H), 2.54-1.85 (m, 6H), 1.29 (d, J=8.0 Hz, 9H). LCMS (ESI): m/z=497 [M+H]⁺.

### xxxv. SRI-030271 (CL-16617-23-2)

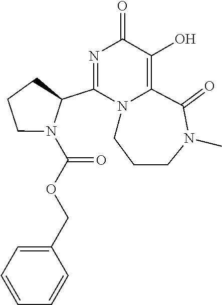 [SMILES: CN1CCCn2c([C@@H]3CCCN3C(=O)OCc3ccccc3)nc(=O)c(O)c2C1=O]

¹H NMR (399 MHz, Methanol-d₄) δ 7.32 (d, J=4.4 Hz, 2H), 7.29-7.22 (m, 2H), 7.13-7.06 (m, 1H), 5.17 (d, J=11.8 Hz, 1H), 5.05-4.93 (m, 1H), 4.75 (d, J=11.8 Hz, 1H), 3.81-3.34 (m, 6H), 3.02 (s, 3H), 2.51-2.38 (m, 2H), 2.34-1.86 (m, 4H). LCMS (ESI): m/z=413 [M+H]⁺.

### xxxvi. SRI-030651 (CL-16617-91B)

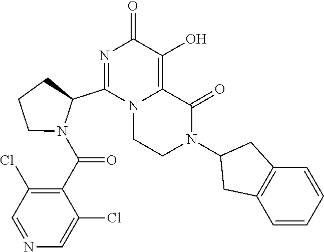 [SMILES: O=C1c2c(O)c(=O)nc([C@@H]3CCCN3C(=O)c3c(Cl)cncc3Cl)n2CCN1C1Cc2ccccc2C1]

¹H NMR (400 MHz, CD₃OD) δ 8.60 (d, J=0.9 Hz, 2H), 7.27-7.16 (m, 4H), 5.55-5.47 (m, 1H), 5.35 (dd, J=8.2, 5.3 Hz, 1H), 4.67-4.61 (m, 1H), 4.46-4.31 (m, 1H), 3.71-3.56 (m, 3H), 3.43-3.31 (m, 3H), 3.14 (dt, J=16.6, 5.9 Hz, 2H), 2.45 (dq, J=13.9, 7.6, 7.1 Hz, 1H), 2.37-2.17 (m, 2H), 2.06-1.94 (m, 1H). LCMS (ESI): m/z=540 [M+H]⁺.

### xxxvii. SRI-030274

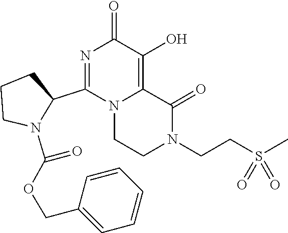 [SMILES: CS(=O)(=O)CCN1CCn2c([C@@H]3CCCN3C(=O)OCc3ccccc3)nc(=O)c(O)c2C1=O]

¹H NMR (400 MHz, CD₃OD): δ 1.85-2.08 (m, 2H), 2.09-2.22 (m, 1H), 2.36 (dt, 1H, J=12.2, 7.4 Hz), 3.06 (d, 3H, J=2.7 Hz), 3.37-4.24 (m, 8H), 4.25-4.35 (m, 1H), 4.5-4.64 (m, 1H), 4.73 (d, 1H, J=11.6 Hz), 5.00-5.09 (m, 1H), 5.14 (d, 1H, J=11.6 Hz), 7.02-7.12 (m, 1H), 7.22-7.28 (m, 2H), 7.31-7.35 (m, 2H). MS (ESI): m/z 491 [M+H]⁺.

### xxxviii. SRI-030275

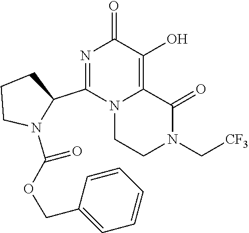 [SMILES: O=C1c2c(O)c(=O)nc([C@@H]3CCCN3C(=O)OCc3ccccc3)n2CCN1CC(F)(F)F]

¹H NMR (400 MHz, CD₃OD): δ 1.85-2.23 (m, 3H), 2.30-2.45 (m, 1H), 3.48-3.63 (m, 2H), 3.64-3.82 (m, 2H), 4.16-4.42 (m, 4H), 4.72 (d, 1H, J=11.5 Hz), 4.95-5.10 (m, 1H), 5.13 (d, 1H, J=11.5 Hz), 7.03-7.11 (m, 1H), 7.18-7.25 (m, 2H), 7.29-7.38 (m, 2H). MS (ESI): m/z 467 [M+H]⁺.

### xxxix. SRI-03095

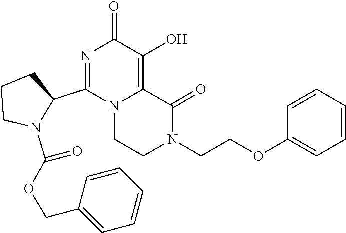 [SMILES: O=C1c2c(O)c(=O)nc([C@@H]3CCCN3C(=O)OCc3ccccc3)n2CCN1CCOc1ccccc1]

¹H NMR (400 MHz, CD₃OD): δ 1.86-2.10 (m, 2H), 2.09-2.24 (m, 1H), 2.35 (dt, 1H, J=12.4, 7.4 Hz), 3.48 (ddd, 1H, J=13.6, 7.4, 3.7 Hz), 3.59 (ddt, 1H, J=10.2, 7.3, 3.5 Hz), 3.65-3.85 (m, 2H), 3.89-4.06 (m, 2H), 4.10-4.33 (m, 3H), 4.49-4.62 (m, 1H), 4.66 (d, 1H, J=11.5 Hz), 5.00 (dd, 1H, J=8.0, 5.3 Hz), 5.04-5.15 (m, 2H), 6.90-7.01 (m, 4H), 7.03-7.14 (m, 2H), 7.21-7.39 (m, 4H). MS (ESI): m/z 505 [M+H]⁺.

### xl. SRI-03096

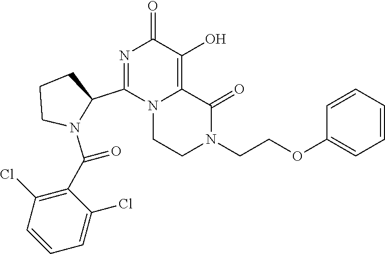 [SMILES: O=C1c2c(O)c(=O)nc([C@@H]3CCCN3C(=O)c3c(Cl)cccc3Cl)n2CCN1CCOc1ccccc1]

¹H NMR (400 MHz, CD₃OD): δ 2.10 (dq, 1H, J=11.9, 7.2, Hz), 2.21-2.41 (m, 2H), 2.45 (ddd, 1H, J=12.3, 9.2, 6.3 Hz), 3.32-3.43 (m, 1H), 3.59 (dt, 1H, J=10.2, 7.3 Hz), 3.92-4.08 (m, 4H), 4.26 (t, 2H, J=5.2 Hz), 4.44 (dt, 1H, J=13.2, 5.6 Hz), 4.75 (dt, 1H, J=13.1, 5.3 Hz), 5.37 (dd, 1H, J=8.1, 5.2 Hz), 6.93 (ddt, 3H, J=13.5, 7.3, 1.1 Hz), 7.22-7.29 (m, 2H), 7.36-7.48 (m, 3H). MS (ESI): m/z 543 [M+H]⁺.

### c. Synthesis of SRI-032118 and SRI-032119

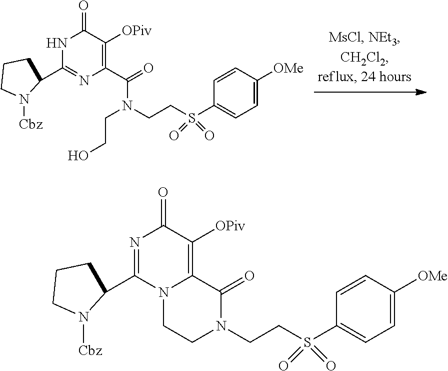

Step 1: To a stirred solution of advanced intermediate (200 mg, 0.292 mmol) dissolved in anhydrous methylene chloride (10 mL) was added mesyl chloride (1.5 eq., 34 μL, 0.438 mmol), followed by triethylamine (3 eq., 122 μL, 0.876 mmol). The mixture was heated under reflux for 24 hours, cooled to room temperature, and concentrated in vacuo. The residue was purified via reverse-phase chromatography, eluting with a water+0.10% formic acid/acetonitrile gradient (5-50% acetonitrile over 30 minutes, and 50-95% over 7 minutes) to give the desired cyclized product (80 mg, 41%) as a white solid after lyophilization. ¹H NMR (300 MHz, CDCl₃): δ 1.35 (s, 6H; Piv), 1.37 (s, 3H; Piv), 1.75-2.04 (m, 2H), 2.19-2.39 (m, 2H), 3.08-3.31 (m, 1H), 3.41-3.66 (m, 3H), 3.73-3.84 (m, 2H), 3.87 (s, 3H; OMe), 3.92-4.27 (m, 3H), 4.73-4.92 (m, 2H), 4.94-5.16 (AB_{q}, 2H; J_{AB}=12.9 Hz, Δν_{AB}=33.1 Hz), 7.02 (d, 2H, J=8.8 Hz), 7.27-7.38 (m, 5H; Cbz), 7.81 (d, 2H, J=8.8 Hz). MS (ESI): m/z 667.17 [M+H]⁺; calculated 666.24 for M. HPLC (water/CH₃CN, 0.1% formic acid; 5-95% acetonitrile over 10 minutes): RT=4.48 minutes.

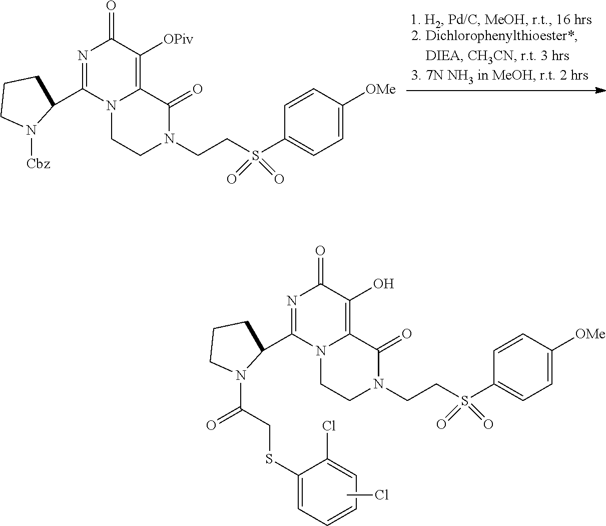

Step 2: To a solution of bicyclic advanced intermediate (80 mg, 0.120 mmol) dissolved in anhydrous methanol (10 mL) was added palladium over carbon (10% loading, 1 eq., 13 mg). The mixture was degassed with stirring under vacuum, and the atmosphere replaced with hydrogen via balloon. The mixture was stirred at room temperature for 16 hours and filtered through celite. The filter cake was washed with methanol (20 mL), and the filtrate was concentrated in vacuo to give deprotected intermediate as an off-white foam (54 mg, 100%), which was used in the next step without further purification.

To a stirred solution of deprotected intermediate (27 mg, 0.0602 mmol) dissolved in anhydrous methylene chloride (5 mL) was added dichlorophenylthioester (1.1 eq, 26 mg), and DIEA (3 eq, 32 μL). The mixture was stirred at room temperature for 3 hours, concentrated, and the residue dissolved in anhydrous methanol (5 mL). A 7N solution of ammonia in methanol (200 μL, 1.4 mmol) was added, and the mixture stirred at room temperature for 2 hours. The reaction was concentrated, and purified via reverse-phase chromatography, eluting with a water+0.1% formic acid/acetonitrile gradient (5-50% acetonitrile over 30 minutes, and 50-95% over 7 minutes) to give the desired product after lyophilization.

### i. SRI-032118

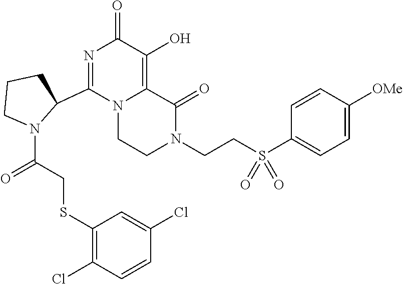 [SMILES: COc1ccc(S(=O)(=O)CCN2CCn3c([C@@H]4CCCN4C(=O)CSc4cc(Cl)ccc4Cl)nc(=O)c(O)c3C2=O)cc1]

11 mg (27%) as a white solid. ¹H NMR (300 MHz, DMSO-d₆): δ 1.86-2.04 (m, 2H), 2.05-2.32 (m, 2H), 3.65-3.74 (m, 4H; exo-pyrimidione —CH₂CH₂—), 3.74-3.82 (m, 4H; —CH₂CH₂— of ethylsulfone), 3.83 (s, 3H), 4.08-4.27 (m, 2H+1H; two signals), 4.36-4.47 (m, 1H), 5.06-5.17 (m, 1H), 7.14 (d, 2H, J=8.8 Hz), 7.21 (dd, 1H, J=8.8, 2.3 Hz), 7.40-7.49 (m, 1H+1H; two signals), 7.84 (d, 2H, J=8.8 Hz). MS (ESI) m/z 666.97 [M+H]⁺, 668.95 [M+2+H]⁺; calculated 666.08. HPLC (water/CH₃CN, 0.1% formic acid; 5-95% acetonitrile over 10 minutes): RT=4.42 minutes.

### ii. SRI-032119

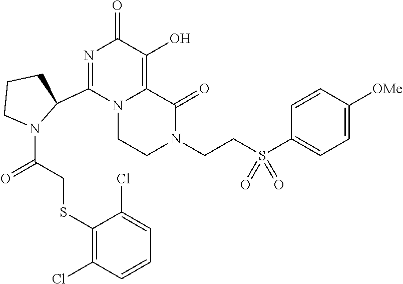 [SMILES: COc1ccc(S(=O)(=O)CCN2CCn3c([C@@H]4CCCN4C(=O)CSc4c(Cl)cccc4Cl)nc(=O)c(O)c3C2=O)cc1]

10 mg (25%) as an off-white solid. ¹H NMR (300 MHz, DMSO-d₆): δ 1.77-2.27 (m, 4H; pyrrolidine —CH₂CH₂—), 3.27-3.55 (m, 4H; exo-pyrimidone —CH₂CH₂—), 3.57-3.97 (m, 4H; —CH₂CH₂— of ethylsulfone; s, 3H; m, 2H; three signals), 4.06-4.18 (m, 1H), 4.32-4.44 (m, 1H), 4.91-5.06 (m, 1H), 7.14 (d, 2H, J=7.6 Hz), 7.40 (t, 1H; J=8.2 Hz), 7.55 (d, 2H, J=8.2 Hz), 7.85 (d, 2H, J=7.6 Hz) MS (ESI) m/z 666.96 [M+H]⁺, 668.94 [M+2+H]⁺; calculated 666.08. HPLC (water/CH₃CN, 0.1% formic acid; 5-95% acetonitrile over 10 minutes): RT=4.33 minutes.

### d. Synthesis of SRI-030269 (4A), SRI-030274 (4B), SRI-030275 (4c), SRI-030292 (4D), and SRI-030395 (4E)

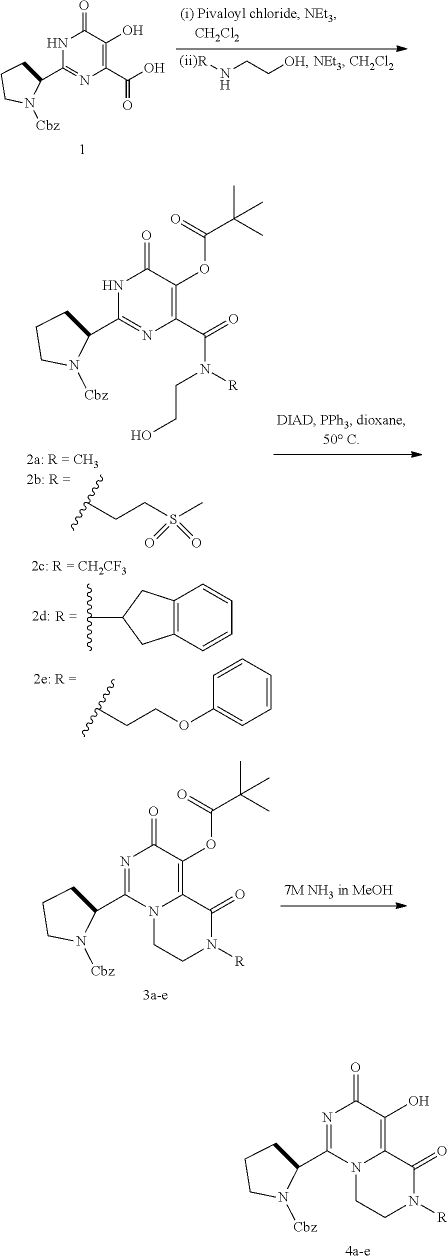

To a stirred mixture of 1 (1 eq.) dissolved in methylene chloride and triethylamine (3 eq.) cooled to 0° C. was added pivaloyl chloride (2 eq.), dropwise. The mixture was stirred overnight, warming up to room temperature. Aminoethanol (1 eq.) was dissolved in methylene chloride and added to the mixture, which was stirred for a further 12 hours. The mixture was concentrated in vacuo, and purified via silica gel chromatography (CH₂Cl₂/0-5% MeOH) to give 2a-e.

To 2a-e (1 eq.) dissolved in 1,4-dioxane was added triphenylphosphine (1.5 eq.) and diisopropylazodicarboxylate (DIAD; 1.4 eq). The mixture was stirred overnight at 50° C. under argon, and reaction progress checked by TLC. The mixture was concentrated in vacuo, and the residue purified by silica gel chromatography (CH₂Cl₂/5-40% acetone) to give 3a-e.

To 3a-e (1 eq.) dissolved in anhydrous methanol was added a 7M ammonia solution in methanol (10 eq.). The mixture was stirred for three hours, and the reaction monitored by LCMS. The mixture was concentrated in vacuo, and purified via reverse-phase chromatography, eluting with a water+0.1% formic acid/acetonitrile gradient (5-50% acetonitrile over 30 minutes, and 50-95% over 7 minutes) to give 4a-e after lyophilization.

### i. SRI-030269 (CL-16617-12-1)

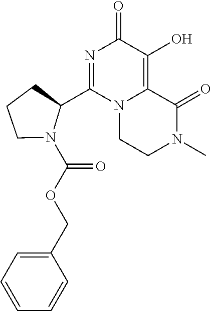 [SMILES: CN1CCn2c([C@@H]3CCCN3C(=O)OCc3ccccc3)nc(=O)c(O)c2C1=O]

¹H NMR (399 MHz, Methanol-d₄) δ 7.39-7.20 (m, 4H), 7.17-7.05 (m, 1H), 5.19-5.03 (m, 3H), 4.69-4.04 (m, 2H), 3.92-3.52 (m, 4H), 3.07 (s, 3H), 2.52-2.36 (m, 1H), 2.21-1.88 (m, 3H). LCMS (ESI): m/z=399 [M+H]⁺.

### ii. SRI-030274 (DJB-16586-95A)

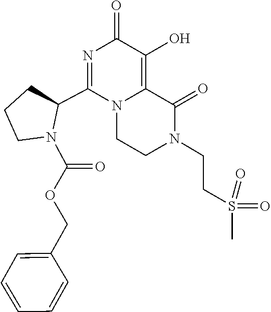 [SMILES: CS(=O)(=O)CCN1CCn2c([C@@H]3CCCN3C(=O)OCc3ccccc3)nc(=O)c(O)c2C1=O]

¹H NMR (400 MHz, Methanol-d₄) δ 7.35-7.31 (m, 2H), 7.28-7.22 (m, 2H), 7.12-7.02 (m, 1H), 5.14 (d, J=11.6 Hz, 1H), 5.09-5.00 (m, 1H), 4.73 (d, J=11.6 Hz, 1H), 4.64-4.52 (m, 1H), 4.35-4.25 (m, 1H), 4.24-3.37 (m, 8H), 3.06 (d, J=2.7 Hz, 3H), 2.36 (dt, J=12.2, 7.4 Hz, 1H), 2.22-2.09 (m, 1H), 2.08-1.85 (m, 2H). LCMS (ESI): m/z=491 [M+H]⁺.

### iii. SRI-030275 (CL-16617-47)

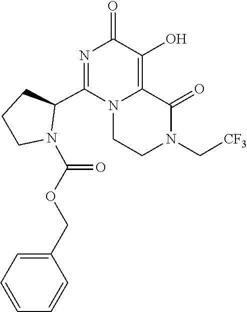 [SMILES: O=C1c2c(O)c(=O)nc([C@@H]3CCCN3C(=O)OCc3ccccc3)n2CCN1CC(F)(F)F]

¹H NMR (399 MHz, Methanol-d₄) δ 7.38-7.29 (m, 2H), 7.25-7.18 (m, 2H), 7.11-7.03 (m, 1H), 5.13 (d, J=11.5 Hz, 1H), 5.10-4.95 (m, 1H), 4.72 (d, J=11.5 Hz, 1H), 4.42-4.16 (m, 4H), 3.82-3.64 (m, 2H), 3.63-3.48 (m, 2H), 2.45-2.30 (m, 1H), 2.23-1.85 (m, 3H). LCMS (ESI): m/z=467 [M+H]⁺.

### iv. SRI-030292 (CL-16617-69)

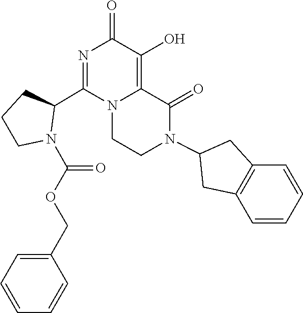 [SMILES: O=C1c2c(O)c(=O)nc([C@@H]3CCCN3C(=O)OCc3ccccc3)n2CCN1C1Cc2ccccc2C1]

¹H NMR (399 MHz, Methanol-d₄) δ 7.40-7.12 (m, 6H), 7.11-7.07 (m, 2H), 7.05-7.03 (m, 1H), 5.53-5.35 (m, 1H), 5.09 (d, J=11.5 Hz, 1H), 5.07-4.91 (m, 1H), 4.72 (d, J=11.5 Hz, 1H), 4.52-4.21 (m, 1H), 4.11-3.87 (m, 1H), 3.83-3.65 (m, 1H), 3.62-3.55 (m, 2H), 3.45-3.22 (m, 3H), 3.16-3.07 (m, 1H), 3.00 (td, J=17.1, 5.2 Hz, 1H), 2.37-2.28 (m, 1H), 2.25-2.10 (m, 1H), 2.06-1.86 (m, 2H). LCMS (ESI): m/z=501 [M+H]⁺.

### v. SRI-030295 (DJB-16644-69-F10)

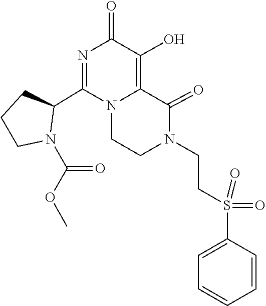 [SMILES: COC(=O)N1CCC[C@H]1c1nc(=O)c(O)c2n1CCN(CCS(=O)(=O)c1ccccc1)C2=O]

¹H NMR (400 MHz, Methanol-d₄) δ 7.99-7.92 (m, 2H), 7.75-7.68 (m, 1H), 7.67-7.58 (m, 2H), 5.14-4.98 (m, 1H), 4.57-4.39 (m, 1H), 4.33-4.16 (m, 1H), 3.93 (t, J=6.4 Hz, 2H), 3.87-3.78 (m, 2H), 3.71-3.61 (m, 5H), 3.59-3.49 (m, 2H), 2.44-2.30 (m, 1H), 2.19-1.86 (m, 3H). LCMS (ESI): m/z=477 [M+H]⁺.

### e. Synthesis of SRI-030272 (6A) and SRI-030273 (6B)

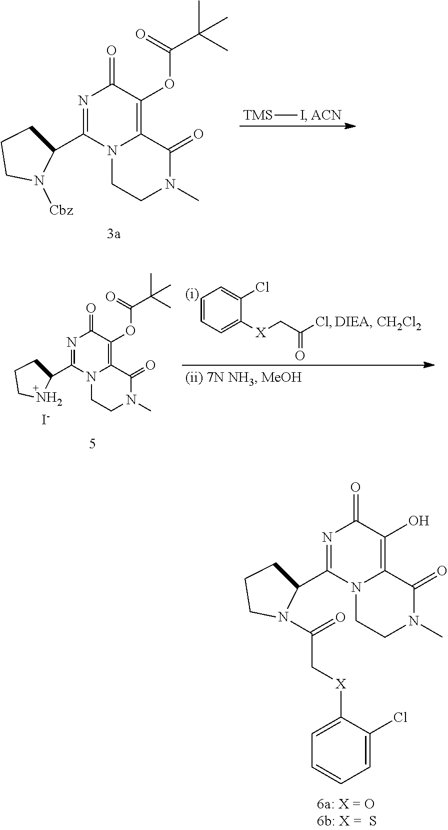

To a solution of 3a (1 eq.) dissolved in anhydrous acetonitrile cooled to 0° C. was added iodotrimethylsilane (3 eq.). The mixture was stirred at 0° C. for 5 min, followed by room temperature for 90 min, and the reaction monitored by LCMS. The mixture was quenched with methanol and concentrated in vacuo to give 5 as a HI salt, which was used in the next step without further purification.

To a solution of 5 (1 eq.) dissolved in anhydrous methylene chloride cooled to 0° C. was added acid chloride (1.3 eq.), followed by DIEA (3 eq.). The mixture was warmed to room temperature and stirred for 2 hours. The mixture was concentrated in vacuo and redissolved in anhydrous methanol. A 7M ammonia solution in methanol (10 eq.) was added, and the mixture was stirred for three hours at room temperature, monitored by LCMS. The mixture was concentrated in vacuo, and purified via reverse-phase chromatography, eluting with a water+0.1% formic acid/acetonitrile gradient (5-50% acetonitrile over 30 minutes, and 50-95% over 7 minutes) to give 6a and 6b after lyophilization.

### i. SRI-030272 (16617-24)

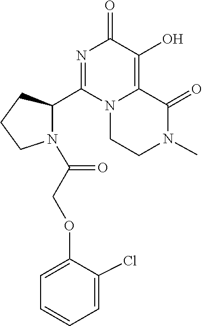 [SMILES: CN1CCn2c([C@@H]3CCCN3C(=O)COc3ccccc3Cl)nc(=O)c(O)c2C1=O]

¹H NMR (399 MHz, Methanol-d₄) δ 7.33 (dd, J=7.9, 1.6 Hz, 1H), 7.21 (ddd, J=8.4, 7.4, 1.6 Hz, 1H), 6.96 (dd, J=8.3, 1.3 Hz, 1H), 6.90 (td, J=7.7, 1.3 Hz, 1H), 5.22 (d, J=6.3 Hz, 1H), 4.91 (dd, 2H); 4.51 (s, 1H), 4.30 (s, 1H), 3.90 (q, J=8.0, 7.0 Hz, 1H), 3.75 (d, J=9.3 Hz, 3H), 3.12 (d, J=1.3 Hz, 3H), 2.47-2.32 (m, 1H), 2.26 (hept, J=6.2, 5.8 Hz, 1H), 2.15-2.00 (m, 2H); MS (ESI): m/z=433 [M+H]⁺.

### ii. SRI-030273 (16617-25)

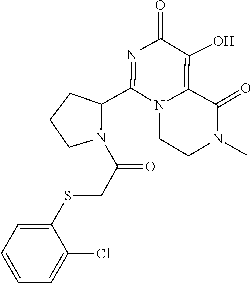 [SMILES: CN1CCn2c(C3CCCN3C(=O)CSc3ccccc3Cl)nc(=O)c(O)c2C1=O]

¹H NMR (399 MHz, Methanol-d₄) δ 7.36-7.28 (m, 2H), 7.26-7.20 (m, 1H), 7.17-7.07 (m, 1H), 5.15 (dd, J=7.8, 4.3 Hz, 1H), 4.59-4.47 (m, 1H), 4.28 (t, J=6.9 Hz, 1H), 3.93 (d, J=5.0 Hz, 3H), 3.83-3.77 (m, 1H), 3.73 (s, 2H), 3.11 (s, 3H), 2.48-2.20 (m, 2H), 2.06 (ddt, J=19.4, 12.1, 6.1 Hz, 2H); MS (ESI): m/z=449 [M+H]⁺.

### f. Synthesis of SRI-030288 (8A), SRI-030289 (8B), and SRI-030390 (8C)

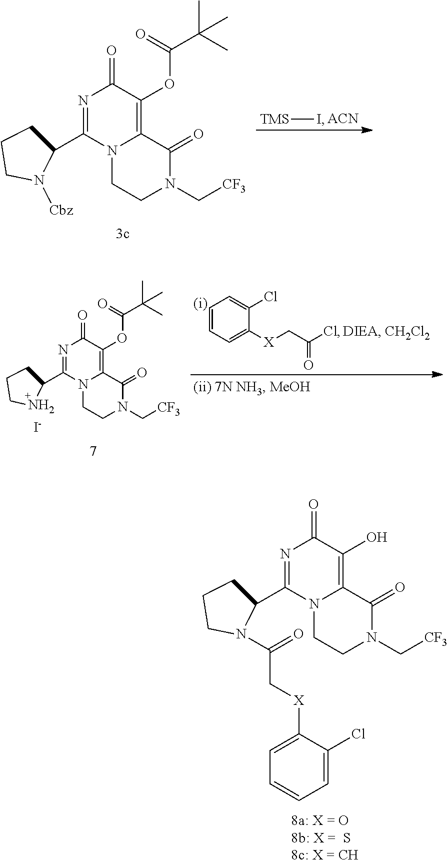

To a solution of 3c (1 eq.) dissolved in anhydrous acetonitrile cooled to 0° C. was added iodotrimethylsilane (3 eq.). The mixture was stirred at 0° C. for 5 min, followed by room temperature for 90 min, and the reaction monitored by LCMS. The mixture was quenched with methanol and concentrated in vacuo to give 7 as a HI salt, which was used in the next step without further purification.

To a solution of 7 (1 eq.) dissolved in anhydrous methylene chloride cooled to 0° C. was added acid chloride (1.3 eq.), followed by DIEA (3 eq.). The mixture was warmed to room temperature and stirred for 2 hours. The mixture was concentrated in vacuo and redissolved in anhydrous methanol. A 7M ammonia solution in methanol (10 eq.) was added, and the mixture was stirred for three hours at room temperature, monitored by LCMS. The mixture was concentrated in vacuo, and purified via reverse-phase chromatography, eluting with a water+0.1% formic acid/acetonitrile gradient (5-50% acetonitrile over 30 minutes, and 50-95% over 7 minutes) to give 8a-c after lyophilization.

### i. SRI-030288 (16617-59)

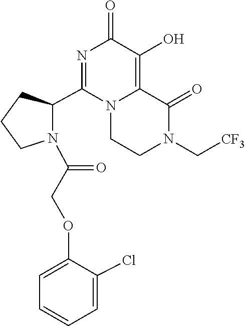 [SMILES: O=C1c2c(O)c(=O)nc([C@@H]3CCCN3C(=O)COc3ccccc3Cl)n2CCN1CC(F)(F)F]

¹H NMR (399 MHz, Methanol-d₄) δ 8.53 (d, J=0.7 Hz, 1H), 7.42-7.22 (m, 2H), 6.96 (d, J=35.4 Hz, 2H), 5.28-5.23 (m, 1H), 4.74 (s, 2H), 4.29 (d, J=33.1 Hz, 3H), 3.89-3.75 (s, 5H), 2.30 (d, J=52.7 Hz, 2H), 2.03 (s, 2H); MS (ESI): m/z=501 [M+H]⁺.

### ii. SRI-030289 (16617-60)

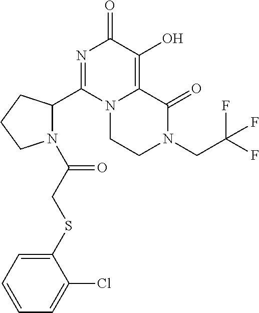 [SMILES: O=C1c2c(O)c(=O)nc(C3CCCN3C(=O)CSc3ccccc3Cl)n2CCN1CC(F)(F)F]

¹H NMR (399 MHz, Methanol-d₄) δ 7.41-7.20 (m, 2H), 7.13 (q, J=8.1, 7.6 Hz, 2H), 5.19 (d, J=32.9 Hz, 1H), 4.57 (s, 1H), 4.43-4.23 (m, 2H), 4.01-3.74 (m, 5H), 3.71-3.65 (m, 2H), 2.48-2.22 (m, 2H), 2.04 (d, J=13.6 Hz, 2H); LCMS (ESI): m/z=517 [M+H]⁺.

### iii. SRI-030290 (16617-62)

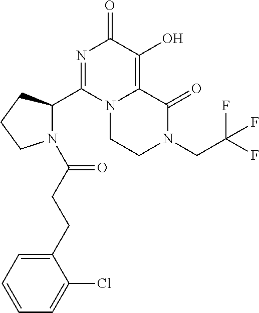 [SMILES: O=C1c2c(O)c(=O)nc([C@@H]3CCCN3C(=O)CCc3ccccc3Cl)n2CCN1CC(F)(F)F]

¹H NMR (399 MHz, Methanol-d₄) δ 7.41-7.25 (m, 2H), 7.25-7.08 (m, 2H), 5.12 (dd, J=8.1, 4.4 Hz, 1H), 4.71-4.53 (m, 1H), 4.45-4.28 (m, 2H), 3.91 (d, J=20.0 Hz, 2H), 3.87-3.72 (m, 1H), 3.71-3.53 (m, 2H), 3.11-2.94 (m, 2H), 2.70 (t, J=7.7 Hz, 2H), 2.27 (ddq, J=41.0, 12.1, 6.4, 5.2 Hz, 2H), 2.00 (ddt, J=32.9, 12.9, 6.4 Hz, 2H). LCMS (ESI): m/z=499 [M+H]⁺.

### g. Synthesis of SRI-030396 (10A), SRI-030423 (10B), SRI-030511 (10C), and SRI-030512 (10D)

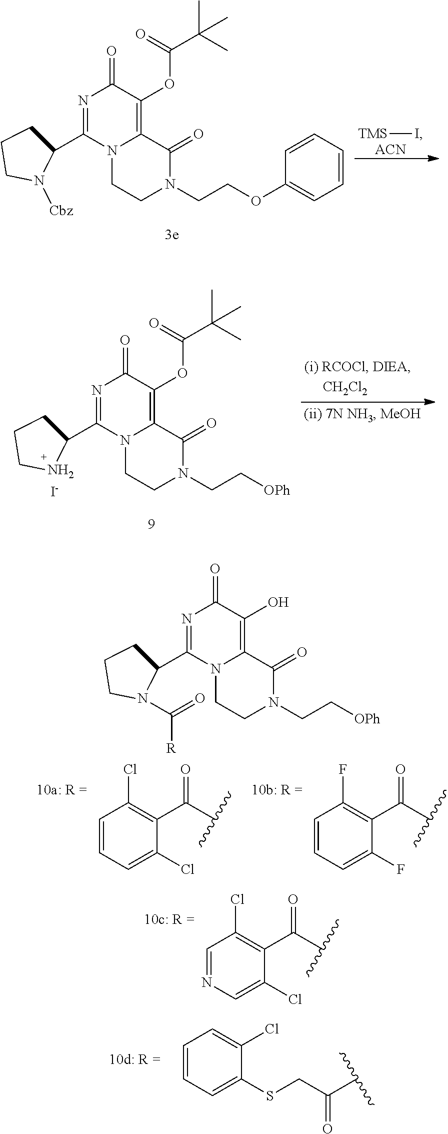

To a solution of 3e (1 eq.) dissolved in anhydrous acetonitrile cooled to 0° C. was added iodotrimethylsilane (3 eq.). The mixture was stirred at 0° C. for 5 min, followed by room temperature for 90 min, and the reaction monitored by LCMS. The mixture was quenched with methanol and concentrated in vacuo to give 9 as a HI salt, which was used in the next step without further purification.

To a solution of 9 (1 eq.) dissolved in anhydrous methylene chloride cooled to 0° C. was added acid chloride (1.3 eq.), followed by DIEA (3 eq.). The mixture was warmed to room temperature and stirred for 2 hours. The mixture was concentrated in vacuo and redissolved in anhydrous methanol. A 7M ammonia solution in methanol (10 eq.) was added, and the mixture was stirred for three hours at room temperature, monitored by LCMS. The mixture was concentrated in vacuo, and purified via reverse-phase chromatography, eluting with a water+0.1% formic acid/acetonitrile gradient (5-50% acetonitrile over 30 minutes, and 50-95% over 7 minutes) to give 10a-d after lyophilization.

### i. SRI-030396 (CL-16617-80)

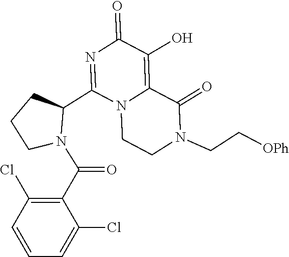 [SMILES: O=C1c2c(O)c(=O)nc([C@@H]3CCCN3C(=O)c3c(Cl)cccc3Cl)n2CCN1CCOc1ccccc1]

¹H NMR (399 MHz, Methanol-d₄) δ 7.48-7.36 (m, 3H), 7.29-7.22 (m, 2H), 6.93 (ddt, J=15.3, 7.3, 1.1 Hz, 3H), 5.37 (dd, J=8.1, 5.2 Hz, 1H), 4.75 (dt, J=13.1, 5.3 Hz, 1H), 4.44 (dt, J=13.2, 5.6 Hz, 1H), 4.26 (t, J=5.2 Hz, 2H), 4.08-3.92 (m, 4H), 3.59 (dt, J=10.2, 7.3 Hz, 1H), 3.43-3.32 (m, 1H), 2.45 (ddd, J=12.3, 9.2, 6.3 Hz, 1H), 2.41-2.21 (m, 2H), 2.00 (dq, J=11.9, 7.2 Hz, 1H). LCMS (ESI): m/z=543 [M+H]⁺.

### ii. SRI-030423 (CL-16617-81)

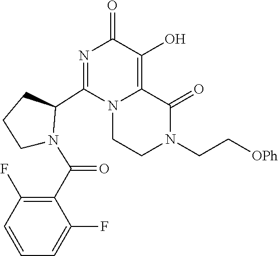 [SMILES: O=C1c2c(O)c(=O)nc([C@@H]3CCCN3C(=O)c3c(F)cccc3F)n2CCN1CCOc1ccccc1]

¹H NMR (399 MHz, Methanol-d₄) δ 7.51 (tt, J=8.5, 6.5 Hz, 1H), 7.35-7.22 (m, 2H), 7.12-7.02 (m, 2H), 6.93 (ddt, J=14.9, 7.4, 1.1 Hz, 3H), 5.36 (dd, J=8.2, 5.0 Hz, 1H), 4.70 (dt, J=11.4, 5.3 Hz, 1H), 4.40 (dt, J=12.5, 5.5 Hz, 1H), 4.27 (t, J=5.1 Hz, 2H), 4.09-3.93 (m, 4H), 3.69 (dt, J=10.2, 7.1 Hz, 1H), 3.53-3.47 (m, 1H), 2.54-2.42 (m, 1H), 2.35-2.14 (m, 2H), 2.08-1.95 (m, 1H). LCMS (ESI): m/z=511 [M+H]⁺.

### iii. SRI-030511 (CL-16617-86B)

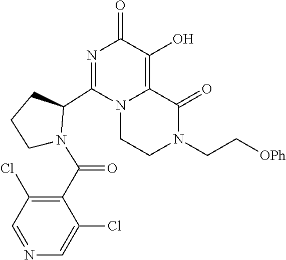 [SMILES: O=C1c2c(O)c(=O)nc([C@@H]3CCCN3C(=O)c3c(Cl)cncc3Cl)n2CCN1CCOc1ccccc1]

¹H NMR (399 MHz, Methanol-d₄) δ 8.61 (d, J=3.9 Hz, 2H), 7.42-7.10 (m, 2H), 7.10-6.87 (m, 3H), 5.40 (dd, J=8.2, 5.2 Hz, 1H), 4.71 (dt, J=13.2, 5.6 Hz, 1H), 4.43 (dt, J=13.1, 5.5 Hz, 1H), 4.27 (t, J=5.2 Hz, 2H), 4.10-3.91 (m, 4H), 3.62 (dt, J=10.1, 7.2 Hz, 1H), 3.46-3.34 (m, 1H), 2.52-2.41 (m, 1H), 2.44-2.18 (m, 2H), 2.11-1.96 (m, 1H). LCMS (ESI): m/z=544 [M+H]⁺.

### iv. SRI-030512 (CL-16617-87B)

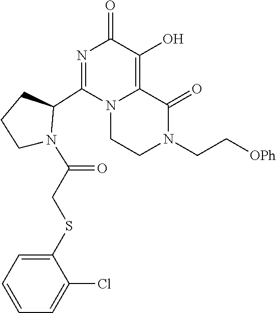 [SMILES: O=C1c2c(O)c(=O)nc([C@@H]3CCCN3C(=O)CSc3ccccc3Cl)n2CCN1CCOc1ccccc1]

¹H NMR (399 MHz, Methanol-d₄) δ 7.35-7.19 (m, 5H), 7.10 (td, J=7.6, 1.6 Hz, 1H), 6.92 (dd, J=8.2, 7.2 Hz, 3H), 5.14 (dd, J=7.9, 4.3 Hz, 1H), 4.60-4.49 (m, 2H), 4.31-4.21 (m, 3H), 3.99-3.89 (m, 6H), 3.78 (dt, J=9.7, 6.7 Hz, 1H), 2.4-2.22 (m, 2H), 2.11-1.96 (m, 2H). LCMS (ESI): m/z=555 [M+H]⁺.

### h. Synthesis of SRI-030649 (12A), SRI-030650 (12B), SRI-030651 (12c), and SRI-030652 (12D)

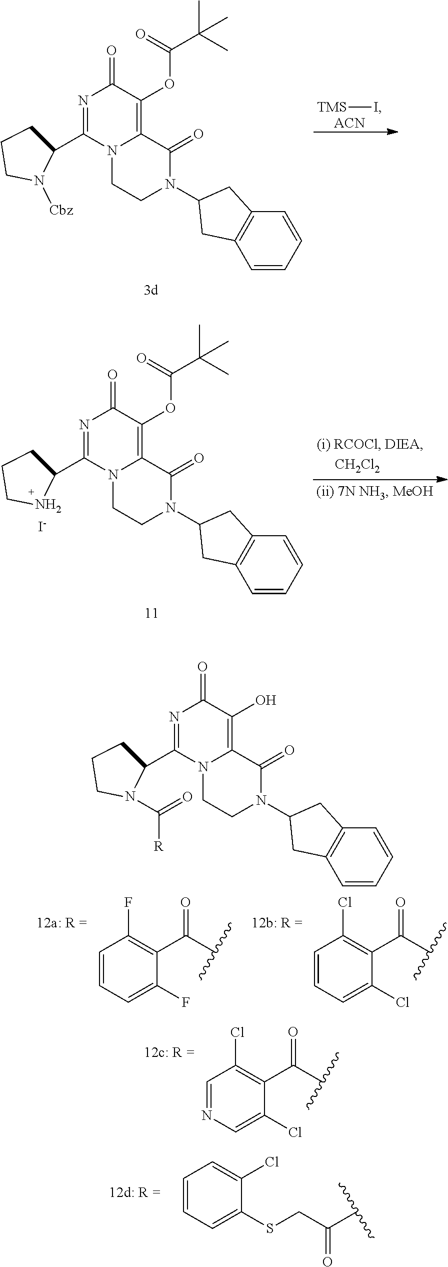

To a solution of 3d (1 eq.) dissolved in anhydrous acetonitrile cooled to 0° C. was added iodotrimethylsilane (3 eq.). The mixture was stirred at 0° C. for 5 min, followed by room temperature for 90 min, and the reaction monitored by LCMS. The mixture was quenched with methanol and concentrated in vacuo to give 11 as a HI salt, which was used in the next step without further purification.

To a solution of 11 (1 eq.) dissolved in anhydrous methylene chloride cooled to 0° C. was added acid chloride (1.3 eq.), followed by DIEA (3 eq.). The mixture was warmed to room temperature and stirred for 2 hours. The mixture was concentrated in vacuo and redissolved in anhydrous methanol. A 7M ammonia solution in methanol (10 eq.) was added, and the mixture was stirred for three hours at room temperature, monitored by LCMS. The mixture was concentrated in vacuo, and purified via reverse-phase chromatography, eluting with a water+0.1% formic acid/acetonitrile gradient (5-50% acetonitrile over 30 minutes, and 50-95% over 7 minutes) to give 12a-d after lyophilization.

### i. SRI-030649 (CL-16617-89B)

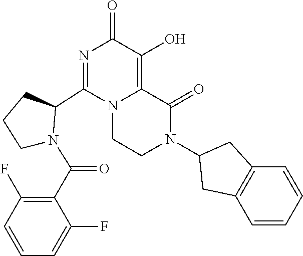 [SMILES: O=C1c2c(O)c(=O)nc([C@@H]3CCCN3C(=O)c3c(F)cccc3F)n2CCN1C1Cc2ccccc2C1]

¹H NMR (399 MHz, Methanol-d₄) δ 7.50 (tt, J=8.4, 6.4 Hz, 1H), 7.21 (ddd, J=33.9, 5.5, 3.2 Hz, 4H), 7.06 (t, J=8.6 Hz, 2H), 5.50 (qd, J=8.1, 5.3 Hz, 1H), 5.31 (dd, J=8.2, 5.0 Hz, 1H), 4.63 (dt, J=11.6, 5.3 Hz, 1H), 4.33 (dt, J=12.8, 5.5 Hz, 1H), 3.75-3.59 (m, 2H), 3.52-3.42 (m, 1H), 3.35-3.22 (m, 3H), 3.13 (dt, J=16.6, 6.0 Hz, 2H), 2.43 (dq, J=14.3, 7.3 Hz, 1H), 2.32-2.15 (m, 2H), 2.08-1.95 (m, 1H). LCMS (ESI): m/z=507 [M+H]⁺.

### ii. SRI-030650 (CL-16617-90B)

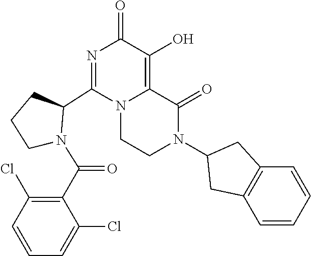 [SMILES: O=C1c2c(O)c(=O)nc([C@@H]3CCCN3C(=O)c3c(Cl)cccc3Cl)n2CCN1C1Cc2ccccc2C1]

¹H NMR (399 MHz, Methanol-d₄) δ 7.55-7.33 (m, 3H), 7.26-7.14 (m, 4H), 5.50 (tt, J=8.1, 5.5 Hz, 1H), 5.31 (dd, J=8.1, 5.3 Hz, 1H), 4.71-4.64 (m, 1H), 4.36 (dt, J=12.3, 5.2 Hz, 1H), 3.64 (t, J=5.5 Hz, 2H), 3.58 (dt, J=10.2, 7.2 Hz, 1H), 3.43-3.25 (m, 3H), 3.13 (dt, J=16.6, 5.9 Hz, 2H), 2.55-2.37 (m, 1H), 2.38-2.18 (m, 2H), 2.02-1.92 (m, 1H). LCMS (ESI): m/z=539 [M+H]⁺.

### iii. SRI-030652 (CL-16617-92B)

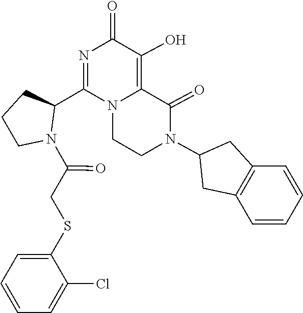 [SMILES: O=C1c2c(O)c(=O)nc([C@@H]3CCCN3C(=O)CSc3ccccc3Cl)n2CCN1C1Cc2ccccc2C1]

¹H NMR (399 MHz, Methanol-d₄) δ 7.35-7.27 (m, 2H), 7.27-7.18 (m, 3H), 7.19-7.13 (m, 2H), 7.13-7.06 (m, 1H), 5.46 (tt, J=8.2, 5.6 Hz, 1H), 5.08 (dd, J=8.0, 4.4 Hz, 1H), 4.44 (dt, J=10.8, 5.1 Hz, 1H), 4.18 (dt, J=12.7, 5.4 Hz, 1H), 3.97-3.88 (s, 3H), 3.76 (dt, J=9.5, 6.5 Hz, 1H), 3.55 (t, J=5.6 Hz, 2H), 3.38-3.21 (m, 2H), 3.08 (dd, J=16.6, 5.5 Hz, 2H), 2.45-2.18 (m, 2H), 2.17-1.94 (m, 2H). LCMS (ESI): m/z=551 [M+H]⁺.

### i. Synthesis of SRI-031014 (16A), SRI-031016 (17B), SRI-031018 (16c), SRI-031020 (16B), SRI-031022 (16D), SRI-030125 (17E), SRI-031026 (17F), and SRI-031029 (16E)

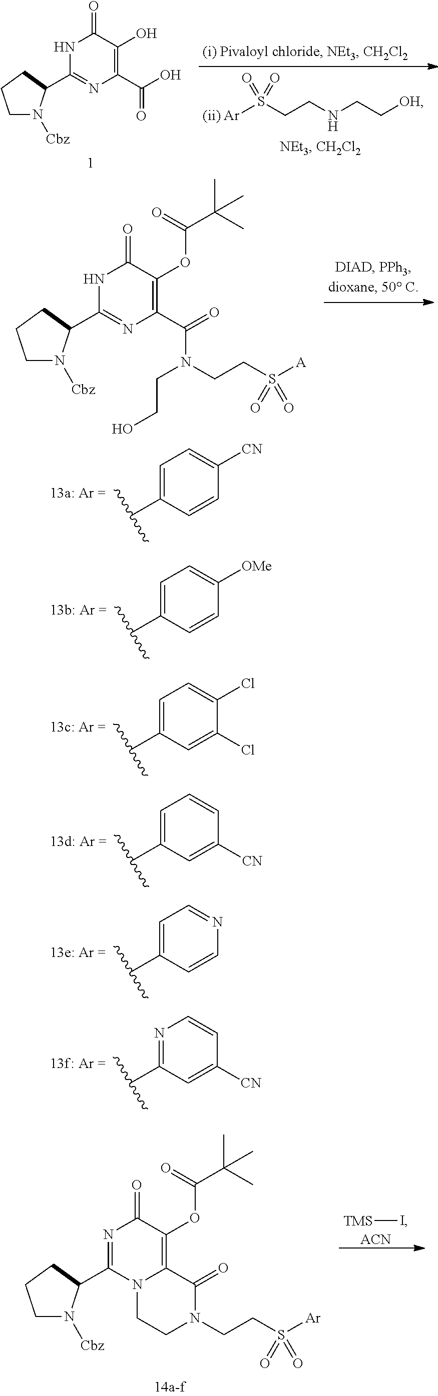

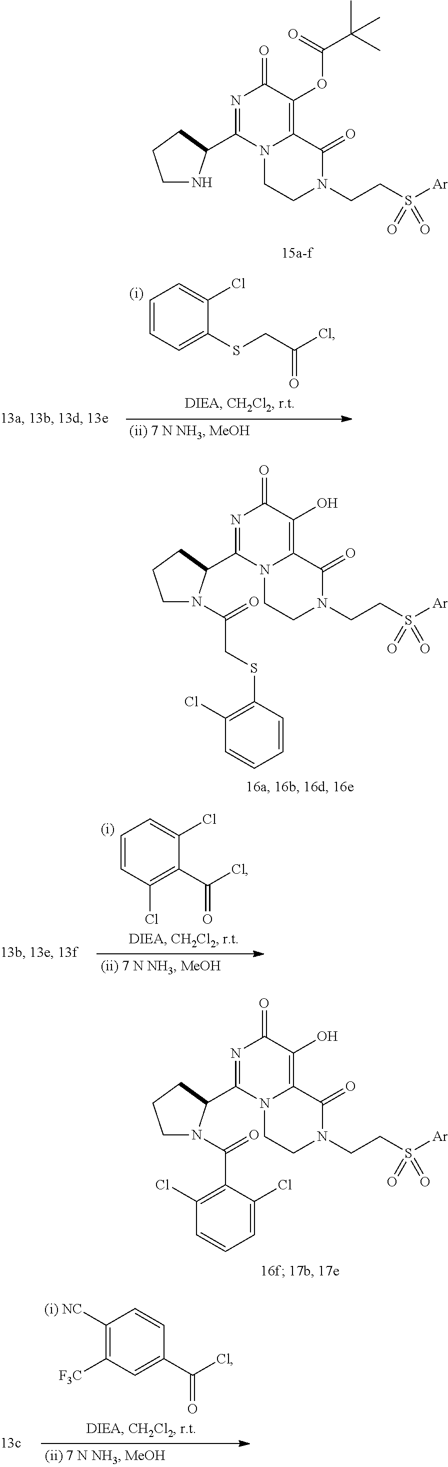

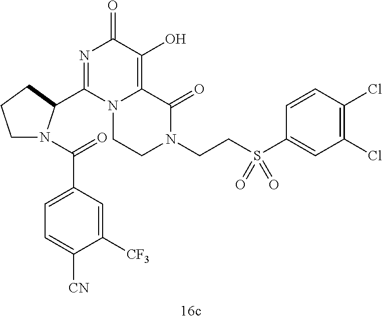 [SMILES: N#Cc1ccc(C(=O)N2CCC[C@H]2c2nc(=O)c(O)c3n2CCN(CCS(=O)(=O)c2ccc(Cl)c(Cl)c2)C3=O)cc1C(F)(F)F]

All compounds were prepared from 1 and purified via reverse-phase chromatography as described above.

### i. SRI-031014

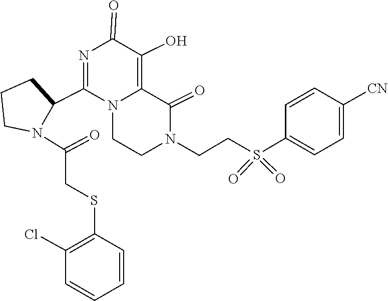 [SMILES: N#Cc1ccc(S(=O)(=O)CCN2CCn3c([C@@H]4CCCN4C(=O)CSc4ccccc4Cl)nc(=O)c(O)c3C2=O)cc1]

### ii. SRI-31016 (GK-16580-36B)

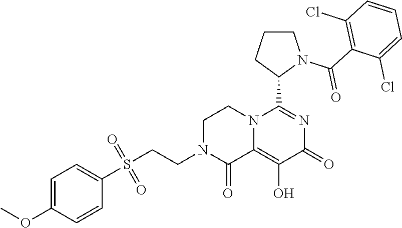 [SMILES: COc1ccc(S(=O)(=O)CCN2CCn3c([C@@H]4CCCN4C(=O)c4c(Cl)cccc4Cl)nc(=O)c(O)c3C2=O)cc1]

¹H NMR (400 MHz, CD₃OD): δ 7.88 (d, 2H), 7.48-7.38 (m, 3H), 7.11 (d, 2H), 5.38 (m, 1H), 4.72 (m, 1H), 4.39 (m, 1H), 3.93 (m, 2H), 3.90 (m, 5H), 3.65-3.58 (m, 3H), 3.37 (m, 1H), 2.45 (m, 1H), 2.36 (m, 1H), 2.23 (m, 1H), 1.99 (m, 1H). MS (ESI+): 621.00.

### iii. SRI-31018 (GK-16580-62C)

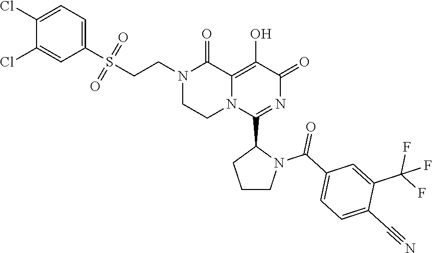 [SMILES: N#Cc1ccc(C(=O)N2CCC[C@H]2c2nc(=O)c(O)c3n2CCN(CCS(=O)(=O)c2ccc(Cl)c(Cl)c2)C3=O)cc1C(F)(F)F]

¹H NMR (400 MHz, CD₃OD): δ 8.15 (d, 2H), 8.07 (m, 2H), 7.90 (d, 1H), 7.83 (d, 1H), 5.18 (m, 1H), 4.65 (m, 1H), 4.35 (m, 1H), 4.01-3.78 (m, 5H), 3.76 (m, 2H), 3.56 (m, 1H), 2.50 (m, 1H), 2.14 (m, 2H), 2.00 (m, 1H). MS (ESI+) 683.99.

### iv. SRI-31020 (GK-16580-45B)

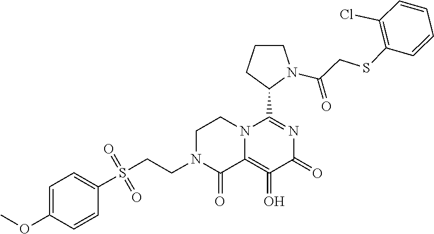

¹H NMR (400 MHz, CD₃OD): δ 7.87 (d, 2H), 7.36-7.21 (m, 3H), 7.13-7.07 (m, 3H) 5.15 (m, 1H), 4.50 (m, 1H), 4.22 (m, 2H), 3.96 (s, 2H), 3.96-3.85 (m, 5H), 3.85 (s, 3H), 3.63-3.57 (m, 2H), 2.37 (m, 1H), 2.25 (m, 1H), 2.04 (m, 2H). MS (ESI+) 633.06.

### v. SRI-031022

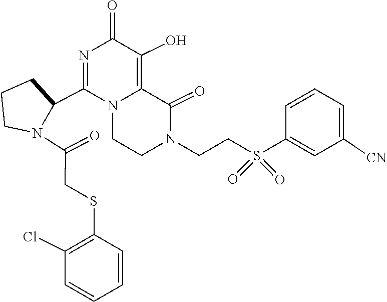 [SMILES: N#Cc1cccc(S(=O)(=O)CCN2CCn3c([C@@H]4CCCN4C(=O)CSc4ccccc4Cl)nc(=O)c(O)c3C2=O)c1]

### vi. SRI-31025 (GK-16580-49B)

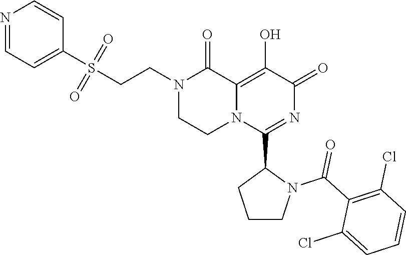 [SMILES: O=C1c2c(O)c(=O)nc([C@@H]3CCCN3C(=O)c3c(Cl)cccc3Cl)n2CCN1CCS(=O)(=O)c1ccncc1]

¹H NMR (400 MHz, CD₃OD): δ 8.91 (d, 2H), 7.97 (d, 2H), 7.46-7.37 (m, 3H), 5.28 (m, 1H), 4.79 (m, 1H), 4.43 (m, 1H), 4.00-3.86 (m, 4H), 3.78 (m, 2H), 3.60 (m, 1H), 3.37 (m, 1H), 2.46 (m, 1H), 2.34 (m, 1H), 2.25 (m, 1H), 2.01 (m, 1H). MS (ESI+) 591.96.

### vii. SRI-31026 (GK-16580-71C)

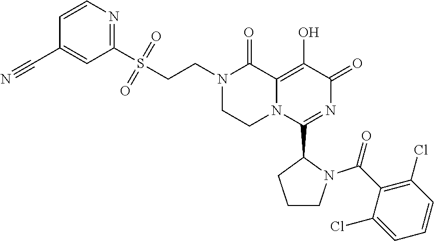 [SMILES: N#Cc1ccnc(S(=O)(=O)CCN2CCn3c([C@@H]4CCCN4C(=O)c4c(Cl)cccc4Cl)nc(=O)c(O)c3C2=O)c1]

¹H NMR (400 MHz, CD₃OD): δ 8.98 (s, 1H), 8.43 (s, 1H), 8.00 (d, 1H), 7.43 (m, 3H), 5.36 (m, 1H), 4.75 (m, 1H), 4.42 (m, 1H), 4.10 (m, 1H), 4.06-3.78 (m, 5H), 3.61 (m, 1H), 3.35 (m, 1H), 2.48 (m, 1H), 2.41-2.20 (m, 2H), 2.01 (m, 1H).

### viii. SRI-31029 (GK-16580-51D)

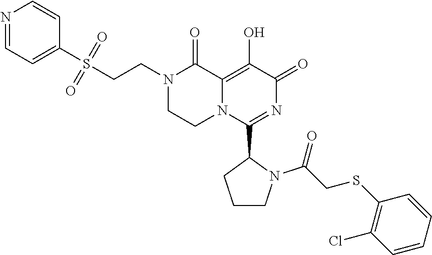 [SMILES: O=C1c2c(O)c(=O)nc([C@@H]3CCCN3C(=O)CSc3ccccc3Cl)n2CCN1CCS(=O)(=O)c1ccncc1]

¹H NMR (400 MHz, CD₃OD): δ 8.91 (d, 2H), 7.95 (d, 2H), 7.36-7.21 (m, 3H), 7.10 (dd, 1H), 5.16 (m, 1H), 4.57 (m, 1H), 4.29 (m, 1H), 3.99-3.90 (m, 5H), 3.90-3.69 (m, 5H), 2.39 (m, 1H), 2.25 (m, 1H), 2.04 (m, 2H). MS (ESI+) 603.10.

### 2. Biological Evaluation of Exemplary Compounds

A list of initial exemplary compounds evaluated for FP binding is shown in Table 1 below.

TABLE 1

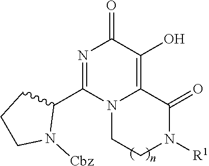

| Compound | | | | |
|---|---|---|---|---|
| No. | R₁ | Stereo | n | FP Kᵢ (μM) |
| SRI-030271 | CH₃ | S | 2 | NE |
| SRI-030269 | CH₃ | S | 1 | 3.584 |
| SRI-030275 | CH₂CF₃ | S | 1 | 3.967 ± 0.148 |
| SRI-030395 | CH₂CH₂OPh | S | 1 | 1.215 ± 0.127 |
| SRI-030292 | 2-Indanyl | S | 1 | 1.952 ± 0.110 |
| SRI-030291 | CH₂CH₂SO₂Ph | S | 2 | 16.767 ± 0.867 |
| SRI-030287 | CH₂CH₂SO₂Ph | S | 1 | 0.112 ± 0.006 |
| SRI-030406 | CH₂CH₂SO₂Ph | R | 1 | 0.175 ± 0.019 |
| SRI-030274 | CH₂CH₂SO₂CH₃ | S | 1 | 4.712 |

A list of the second set of exemplary compounds evaluated for FP binding is shown in Table 2 below.

TABLE 2

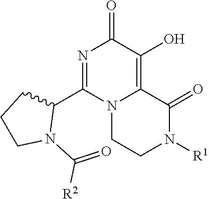

| Compound | R₁ | R₂ | Stereo | FP Kᵢ (μM) |
|---|---|---|---|---|
| SRI-030662 | CH₂CH₂SO₂Ph | CH2S-phenyl-2-Cl | S | X |
| SRI-030405 | CH₂CH₂SO₂Ph | 2,6-Dichlorophenyl | S | 0.079 ± 0.013 |
| SRI-030330 | CH₂CH₂SO₂Ph | 2,6-Difluorophenyl | S | 0.067 ± 0.039 |
| SRI-030407 | CH₂CH₂SO₂Ph | 3,5- | S | 0.305 ± 0.049 |
| SRI-030295 | CH₂CH₂SO₂Ph | OCH₃ | S | 0.745 ± 0.066 |
| SRI-030663 | CH₂CH₂SO₂Ph | CH₂S-phenyl-2-C1 | R | X |
| SRI-030526 | CH₂CH₂SO₂Ph | 2,6-Dichlorophenyl | R | 0.082 ± 0.016 |
| SRI-030512 | CH₂CH₂OPh | CH₂S-phenyl-2-Cl | S | 0.549 ± 0.012 |
| SRI-030396 | CH₂CH₂OPh | 2,6-Dichlorophenyl | S | 0.448 ± 0.024 |
| SRI-030423 | CH₂CH₂OPh | 2,6-Difluorophenyl | S | 0.237 ± 0.030 |
| SRI-030511 | CH₂CH₂OPh | 3,5- | S | 0.222 ± 0.038 |
| | | Dichloroisonicotinyl | | |
| SRI-030652 | 2-Indanyl | CH₂S-phenyl-2-Cl | S | X |
| SRI-030650 | 2-Indanyl | 2,6-Dichlorophenyl | S | X |
| SRI-030649 | 2-Indanyl | 2,6-Difluorophenyl | S | X |
| SRI-030651 | 2-Indanyl | 3,5- | S | X |
| | | Dichloroisonicotinyl | | |

A list of exemplary compounds evaluated for potency and binding is shown in Table 3 below.

TABLE 3

| Corp | | | | |
|---|---|---|---|---|
| Reg | Compound | | IC₅₀ | Kᵢ |
| Number | structure | Compound name | (μM) | (μM) |
| SRI- 030330 | 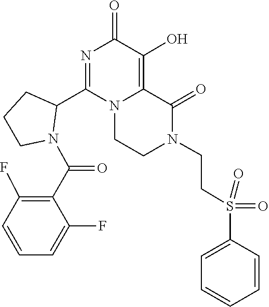 [SMILES: O=C1c2c(O)c(=O)nc(C3CCCN3C(=O)c3c(F)cccc3F)n2CCN1CCS(=O)(=O)c1ccccc1] | 6-(1-(2,6- difluorobenzoyl)pyrrolidin-2- yl)-9-hydroxy-2-(2- (phenylsulfonyl)ethyl)-3,4- dihydro-2H-pyrazino[1,2- c]pyrimidine-1,8-dione | 0.338 ± 0.107 | 0.067 ± 0.039 |
| SRI- 030662 | 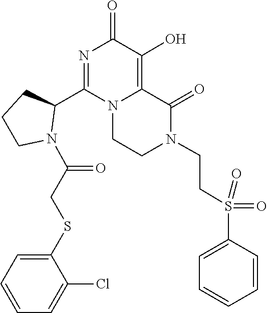 [SMILES: O=C1c2c(O)c(=O)nc([C@@H]3CCCN3C(=O)CSc3ccccc3Cl)n2CCN1CCS(=O)(=O)c1ccccc1] | 6-(1-(2-((2- chlorophenyl)thio)acetyl) pyrrolidin-2-yl)-9-hydroxy- 2-(2-(phenylsulfonyl)ethyl)- 3,4-dihydro-2H-pyrazino [1,2-c]pyrimidine-1,8-dione | 0.352 ± 0.004 | 0.078 ± 0.002 |
| SRI- 030405 | 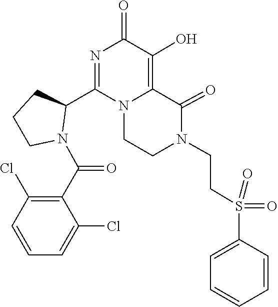 [SMILES: O=C1c2c(O)c(=O)nc([C@@H]3CCCN3C(=O)c3c(Cl)cccc3Cl)n2CCN1CCS(=O)(=O)c1ccccc1] | (S)-6-(1-(2,6- dichlorobenzoyl)pyrrolidin-2- yl)-9-hydroxy-2-(2- (phenylsulfonyl)ethyl)-3,4- dihydro-2H-pyrazino[1,2- c]pyrimidine-1,8-dione | 0.331 ± 0.026 | 0.079 ± 0.013 |
| SRI- 030526 | 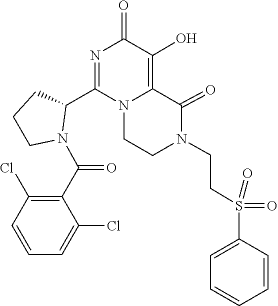 [SMILES: O=C1c2c(O)c(=O)nc([C@H]3CCCN3C(=O)c3c(Cl)cccc3Cl)n2CCN1CCS(=O)(=O)c1ccccc1] | (R)-6-(1-(2,6- dichlorobenzoyl)pyrrolidin-2- yl)-9-hydroxy-2-(2- (phenylsulfonyl)ethyl)-3,4- dihydro-2H-pyrazino[1,2- c]pyrimidine-1,8-dione | 0.380 ± 0.047 | 0.082 ± 0.016 |
| SRI- 030287 | 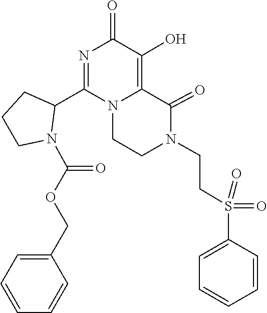 [SMILES: O=C1c2c(O)c(=O)nc(C3CCCN3C(=O)OCc3ccccc3)n2CCN1CCS(=O)(=O)c1ccccc1] | benzyl 2-(9-hydroxy-1,8-dioxo- 2-(2-(phenylsulfonyl)ethyl)- 1,3,4,8-tetrahydro-2H- pyrazino[1,2-c]pyrimidin-6- yl)pyrrolidine-1-carboxylate | 0.407 ± 0.015 | 0.112 ± 0.006 |
| SRI- 030663 | 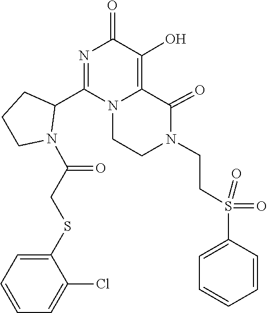 [SMILES: O=C1c2c(O)c(=O)nc(C3CCCN3C(=O)CSc3ccccc3Cl)n2CCN1CCS(=O)(=O)c1ccccc1] | 6-(1-(2-((2- chlorophenyl)thio)acetyl) pyrrolidin-2-yl)-9-hydroxy- 2-(2-(phenylsulfonyl)ethyl)- 3,4-dihydro-2H-pyrazino[1,2- c]pyrimidine-1,8-dione | 0.507 ± 0.027 | 0.139 ± 0.008 |
| SRI- 030406 | 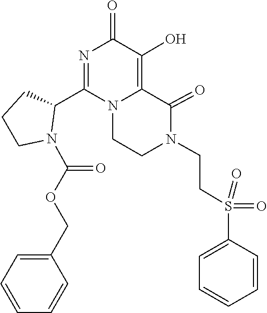 [SMILES: O=C1c2c(O)c(=O)nc([C@H]3CCCN3C(=O)OCc3ccccc3)n2CCN1CCS(=O)(=O)c1ccccc1] | (R)-9-hydroxy-6-(1-(2- phenoxyacetyl)pyrrolidin-2-yl)- 2-(2-(phenylsulfonyl)ethyl)-3,4- dihydro-2H-pyrazino[1,2- c]pyrimidine-1,8-dione | 0.554 ± 0.044 | 0.175 ± 0.019 |
| SRI- 030511 | 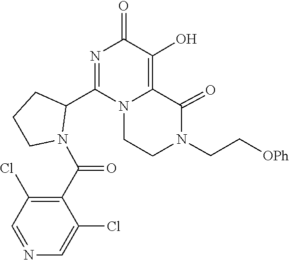 [SMILES: O=C1c2c(O)c(=O)nc(C3CCCN3C(=O)c3c(Cl)cncc3Cl)n2CCN1CCOc1ccccc1] | 6-(1-(3,5- dichloroisonicotinoyl)pyrrolidin- 2-yl)-9-hydroxy-2-(2- phenoxyethyl)-3,4-dihydro-2H- pyrazino[1,2-c]pyrimidine-1,8- dione | 0.770 ± 0.107 | 0.222 ± 0.038 |
| SRI- 030423 | 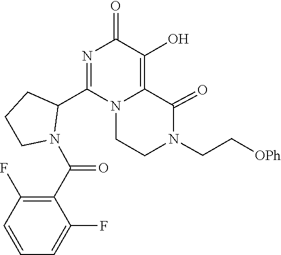 [SMILES: O=C1c2c(O)c(=O)nc(C3CCCN3C(=O)c3c(F)cccc3F)n2CCN1CCOc1ccccc1] | 6-(1-(2,6- difluorobenzoyl)pyrrolidin-2- yl)-9-hydroxy-2-(2- phenoxyethyl)-3,4-dihydro-2H- pyrazino[1,2-c]pyrimidine-1,8- dione | 0.814 ± 0.083 | 0.237 ± 0.030 |
| SRI- 030407 | 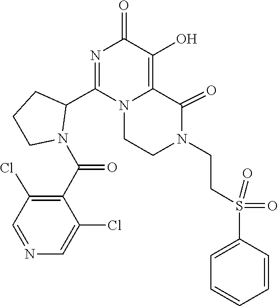 [SMILES: O=C1c2c(O)c(=O)nc(C3CCCN3C(=O)c3c(Cl)cncc3Cl)n2CCN1CCS(=O)(=O)c1ccccc1] | 6-(1-(3,5- dichloroisonicotinoyl)pyrrolidin- 2-yl)-9-hydroxy-2-(2- (phenylsulfonyl)ethyl)-3,4- dihydro-2H-pyrazino[1,2- c]pyrimidine-1,8-dione | 0.857 ± 0.113 | 0.305 ± 0.049 |
| SRI- 030396 | 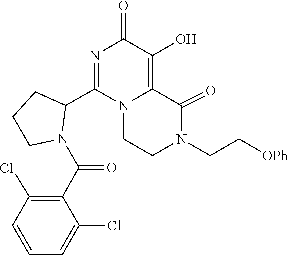 [SMILES: O=C1c2c(O)c(=O)nc(C3CCCN3C(=O)c3c(Cl)cccc3Cl)n2CCN1CCOc1ccccc1] | 6-(1-(2,6- dichlorobenzoyl)pyrrolidin-2- yl)-9-hydroxy-2-(2- phenoxyethyl)-3,4-dihydro-2H- pyrazino[1,2-c]pyrimidine-1,8- dione | 1.190 ± 0.055 | 0.448 ± 0.024 |
| SRI- 030512 | 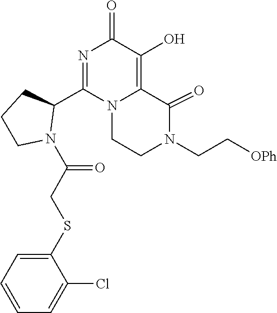 [SMILES: O=C1c2c(O)c(=O)nc([C@@H]3CCCN3C(=O)CSc3ccccc3Cl)n2CCN1CCOc1ccccc1] | 6-(1-(2-((2- chlorophenyl)thio)acetyl) pyrrolidin-2-yl)-9-hydroxy-2-(2- phenoxyethyl)-3,4-dihydro-2H- pyrazino[1,2-c]pyrimidine-1,8- dione | 1.677 ± 0.034 | 0.549 ± 0.012 |
| SRI- 030295 | 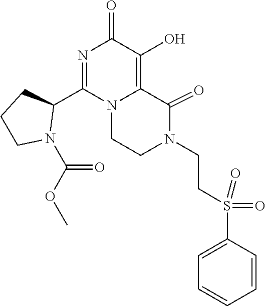 [SMILES: COC(=O)N1CCC[C@H]1c1nc(=O)c(O)c2n1CCN(CCS(=O)(=O)c1ccccc1)C2=O] | methyl 2-(9-hydroxy-1,8-dioxo- 2-(2-(phenylsulfonyl)ethyl)- 1,3,4,8-tetrahydro-2H- pyrazino[1,2-c]pyrimidin-6- yl)pyrrolidine-1-carboxylate | 1.824 ± 0.147 | 0.689 ± 0.060 |
| SRI- 030395 | 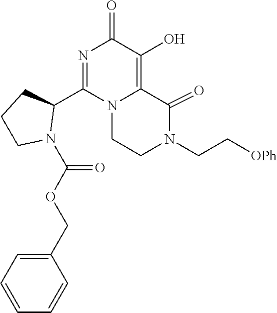 [SMILES: O=C1c2c(O)c(=O)nc([C@@H]3CCCN3C(=O)OCc3ccccc3)n2CCN1CCOc1ccccc1] | benzyl 2-(9-hydroxy-1,8-dioxo- 2-(2-phenoxyethyl)-1,3,4,8- tetrahydro-2H-pyrazino[1,2- c]pyrimidin-6-yl)pyrrolidine-1- carboxylate | 2.974 ± 0.127 | 1.215 ± 0.127 |
| SRI- 030273 | 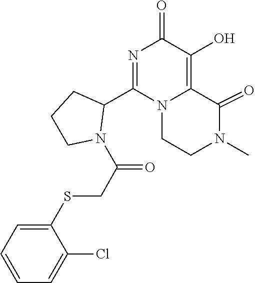 [SMILES: CN1CCn2c(C3CCCN3C(=O)CSc3ccccc3Cl)nc(=O)c(O)c2C1=O] | 6-(1-(2-((2- chlorophenyl)thio)acetyl) pyrrolidin-2-yl)-9-hydroxy- 2-methyl-3,4-dihydro-2H- pyrazino[1,2-c] pyrimidine-1,8-dione | 4.57 ± 0.273 | 1.596 ± 0.099 |
| SRI- 030649 | 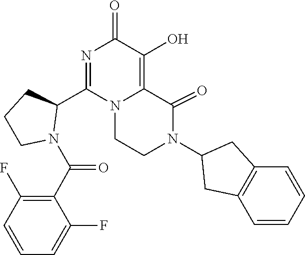 [SMILES: O=C1c2c(O)c(=O)nc([C@@H]3CCCN3C(=O)c3c(F)cccc3F)n2CCN1C1Cc2ccccc2C1] | 6-(1-(2,6- difluorobenzoyl)pyrrolidin-2- yl)-2-(2,3-dihydro-1H-inden-2- yl)-9-hydroxy-3,4-dihydro-2H- pyrazino[1,2-c]pyrimidine-1,8- dione | 4.752 ± 0.225 | 1.789 ± 0.088 |
| SRI- 030292 | 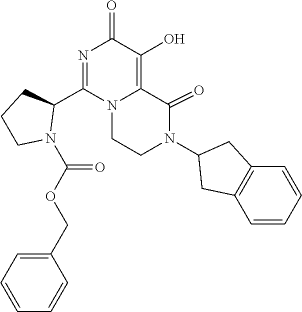 [SMILES: O=C1c2c(O)c(=O)nc([C@@H]3CCCN3C(=O)OCc3ccccc3)n2CCN1C1Cc2ccccc2C1] | benzyl 2-(2-(2,3-dihydro-1H- inden-2-yl)-9-hydroxy-1,8- dioxo-1,3,4,8-tetrahydro-2H- pyrazino[1,2-c]pyrimidin-6- yl)pyrrolidine-1-carboxylate | 4.954 ± 0.742 | 1.977 ± 0.305 |
| SRI- 030271 | 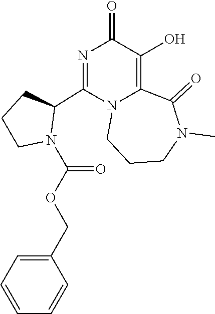 [SMILES: CN1CCCn2c([C@@H]3CCCN3C(=O)OCc3ccccc3)nc(=O)c(O)c2C1=O] | benzyl 2-(10-hydroxy-2-methyl- 1,9-dioxo-1,2,3,4,5,9- hexahydropyrimido[1,6- a][1,4]diazepin-7-yl)pyrrolidine- 1-carboxylate | 35.368 ± 5.364 | 12.726 ± 1.938 |
| SRI- 030291 | 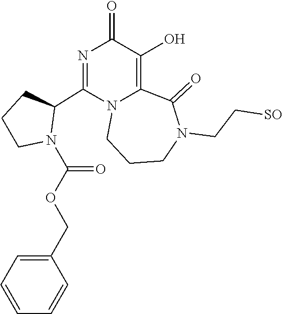 | benzyl 2-(10-hydroxy-1,9-dioxo- 2-(2-(phenylsulfonyl)ethyl)- 1,2,3,4,5,9- hexahydropyrimido[1,6- a][1,4]diazepin-7-yl)pyrrolidine- 1-carboxylate | 39.156 ± 2.017 | 16.767± 0.867 |
| SRI- 030288 | 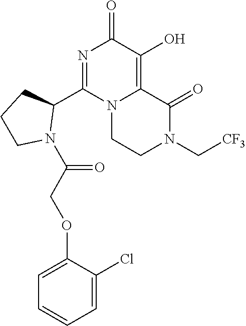 [SMILES: O=C1c2c(O)c(=O)nc([C@@H]3CCCN3C(=O)COc3ccccc3Cl)n2CCN1CC(F)(F)F] | 2-chlorophenyl 2-(9-hydroxy- 1,8-dioxo-2-(2,2,2- trifluoroethyl)-1,3,4,8- tetrahydro-2H-pyrazino[1,2- c]pyrimidin-6-yl)pyrrolidine-1- carboxylate | 9.132 ± 3.786 | 3.232 ± 1.362 |
| SRI- 030272 | 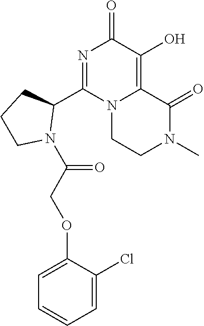 [SMILES: CN1CCn2c([C@@H]3CCCN3C(=O)COc3ccccc3Cl)nc(=O)c(O)c2C1=O] | 2-chlorophenyl 2-(9-hydroxy-2- methyl-1,8-dioxo-1,3,4,8- tetrahydro-2H-pyrazino[1,2- c]pyrimidin-6-yl)pyrrolidine-1- carboxylate | 9.196 ± 0.625 | 3.268 ± 0.226 |
| SRI- 030650 | 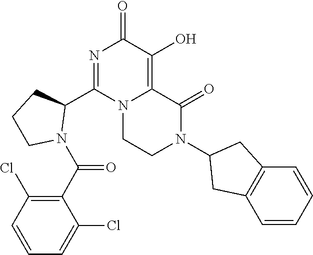 [SMILES: O=C1c2c(O)c(=O)nc([C@@H]3CCCN3C(=O)c3c(Cl)cccc3Cl)n2CCN1C1Cc2ccccc2C1] | 2,6-dichlorophenyl 2-(2-(2,3- dihydro-1H-inden-2-yl)-9- hydroxy-1,8-dioxo-1,3,4,8- tetrahydro-2H-pyrazino[1,2- c]pyrimidin-6-yl)pyrrolidine-1- carboxylate | 8.870 ± 1.321 | 3.390 ± 0.514 |
| SRI- 030269 | 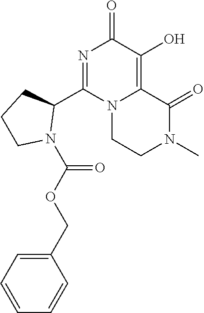 [SMILES: CN1CCn2c([C@@H]3CCCN3C(=O)OCc3ccccc3)nc(=O)c(O)c2C1=O] | benzyl 2-(9-hydroxy-2-methyl- 1,8-dioxo-1,3,4,8-tetrahydro- 2H-pyrazino[1,2-c]pyrimidin-6- yl)pyrrolidine-1-carboxylate | 8.372 ± 1.537 | 3.429 ± 0.639 |
| SRI- 030275 | 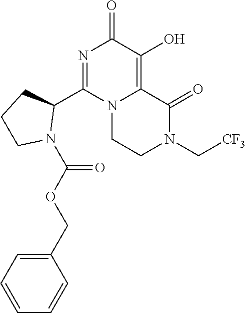 [SMILES: O=C1c2c(O)c(=O)nc([C@@H]3CCCN3C(=O)OCc3ccccc3)n2CCN1CC(F)(F)F] | benzyl 2-(9-hydroxy-1,8-dioxo- 2-(2,2,2-trifluoroethyl)-1,3,4,8- tetrahydro-2H-pyrazino[1,2- c]pyrimidin-6-yl)pyrrolidine-1- carboxylate | 9.376 ± 0.344 | 3.967 ± 0.148 |
| SRI- 030652 | 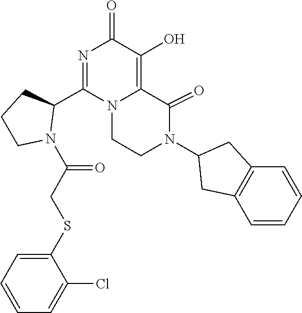 [SMILES: O=C1c2c(O)c(=O)nc([C@@H]3CCCN3C(=O)CSc3ccccc3Cl)n2CCN1C1Cc2ccccc2C1] | 6-(1-(2-((2- chlorophenyl)thio)acetyl) pyrrolidin-2-yl)-2-(2,3- dihydro-1H-inden-2-yl)- 9-hydroxy-3,4- dihydro-2H-pyrazino[1,2- c]pyrimidine-1,8-dione | 10.364 ± 2.367 | 3.971 ± 0.921 |
| SRI- 030289 | 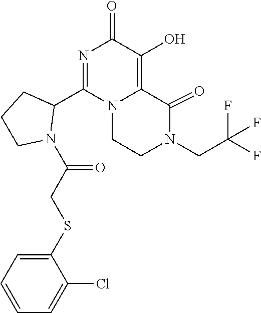 [SMILES: O=C1c2c(O)c(=O)nc(C3CCCN3C(=O)CSc3ccccc3Cl)n2CCN1CC(F)(F)F] | 6-(1-(2-((2- chlorophenyl)thio)acetyl) pyrrolidin-2-yl)-9-hydroxy- 2-(2,2,2-trifluoroethyl)-3,4- dihydro-2H-pyrazino[1,2- c]pyrimidine-1,8- dione | 11.856 ± 0.297 | 4.229 ± 0.107 |
| SRI- 030274 | 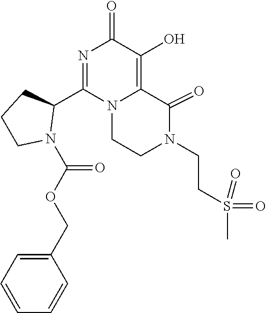 [SMILES: CS(=O)(=O)CCN1CCn2c([C@@H]3CCCN3C(=O)OCc3ccccc3)nc(=O)c(O)c2C1=O] | benzyl 2-(9-hydroxy-2-(2- (methylsulfonyl)ethyl)-1,8- dioxo-1,3,4,8-tetrahydro-2H- pyrazino[1,2-c]pyrimidin-6- yl)pyrrolidine-1-carboxylate | 13.18 ± 1.046 | 4.708 ± 0.378 |
| SRI- 030651 | 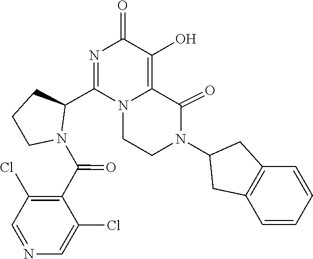 [SMILES: O=C1c2c(O)c(=O)nc([C@@H]3CCCN3C(=O)c3c(Cl)cncc3Cl)n2CCN1C1Cc2ccccc2C1] | 6-(1-(3,5- dichloroisonicotinoyl) pyrrolidin-2-yl)-2-(2,3- dihydro-1H-inden- 2-yl)-9-hydroxy-3,4-dihydro- 2H-pyrazino[1,2-c]pyrimidine- 1,8-dione | 14.381 ± 2.736 | 5.533 ± 1.064 |
| SRI- 030290 | 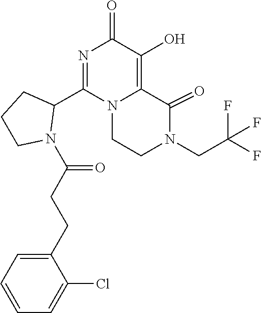 [SMILES: O=C1c2c(O)c(=O)nc(C3CCCN3C(=O)CCc3ccccc3Cl)n2CCN1CC(F)(F)F] | 6-(1-(3-(2- chlorophenyl)propanoyl) pyrrolidin-2-yl)-9-hydroxy- 2-(2,2,2-trifluoroethyl)- 3,4-dihydro-2H-pyrazino [1,2-c]pyrimidine-1,8- dione | 16.828 ± 1.338 | 6.026 ± 0.484 |
| SRI- 031016 | 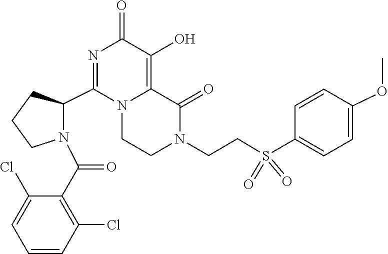 [SMILES: COc1ccc(S(=O)(=O)CCN2CCn3c([C@@H]4CCCN4C(=O)c4c(Cl)cccc4Cl)nc(=O)c(O)c3C2=O)cc1] | (S)-6-(1-(2,6- dichlorobenzoyl)pyrrolidin-2- yl)-9-hydroxy-2-(2-((4- methoxyphenyl)sulfonyl)ethyl)- 3,4-dihydro-2H-pyrazino[1,2- c]pyrimidine-1,8-dione | 0.139 ± 0.007 | −0.004 ± 0.003 |
| SRI- 031020 | 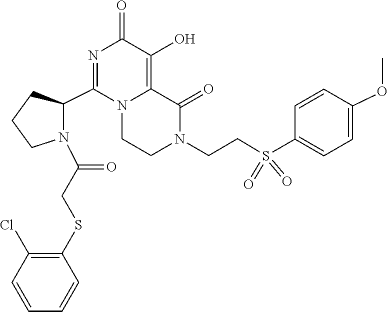 [SMILES: COc1ccc(S(=O)(=O)CCN2CCn3c([C@@H]4CCCN4C(=O)CSc4ccccc4Cl)nc(=O)c(O)c3C2=O)cc1] | (S)-6-(1-(2-((2- chlorophenyl)thio)acetyl) pyrrolidin-2-yl)-9-hydroxy- 2-(2-((4-methoxyphenyl) sulfonyl)ethyl)-3,4-dihydro- 2H-pyrazino[1,2- c]pyrimidine-1,8-dione | 0.132 ± 0.022 | −0.007 ± 0.009 |
| SRI- 031024 | 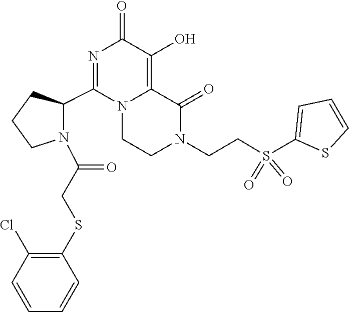 [SMILES: O=C1c2c(O)c(=O)nc([C@@H]3CCCN3C(=O)CSc3ccccc3Cl)n2CCN1CCS(=O)(=O)c1cccs1] | (S)-6-(1-(2-((2- chlorophenyl)thio)acetyl) pyrrolidin-2-yl)-9- hydroxy-2-(2-(thiophen- 2-ylsulfonyl)ethyl)- 3,4-dihydro-2H- pyrazino[1,2- c]pyrimidine-1,8-dione | 0.164 ± 0.052 | 0.007 ± 0.022 |
| | 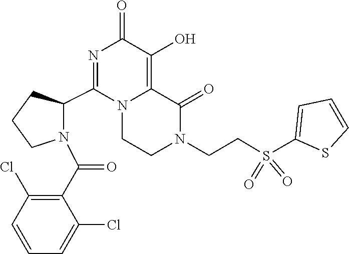 [SMILES: O=C1c2c(O)c(=O)nc([C@@H]3CCCN3C(=O)c3c(Cl)cccc3Cl)n2CCN1CCS(=O)(=O)c1cccs1] | (S)-6-(1-(2,6- dichlorobenzoyl)pyrrolidin-2- yl)-9-hydroxy-2-(2-(thiophen- 2-ylsulfonyl)ethyl)-3,4-dihydro- 2H-pyrazino[1,2-c]pyrimidine- 1,8-dione | 0.250 ± 0.087 | 0.034 ± 0.031 |
| SRI- 031013 | 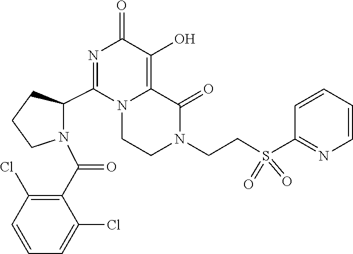 [SMILES: O=C1c2c(O)c(=O)nc([C@@H]3CCCN3C(=O)c3c(Cl)cccc3Cl)n2CCN1CCS(=O)(=O)c1ccccn1] | (S)-6-(1-(2,6- dichlorobenzoyl)pyrrolidin-2- yl)-9-hydroxy-2-(2-(pyridin-2- ylsulfonyl)ethyl)-3,4-dihydro- 2H-pyrazino[1,2-c]pyrimidine- 1,8-dione | 0.204 ± 0.028 | 0.023 ± 0.012 |
| SRI- 031017 | 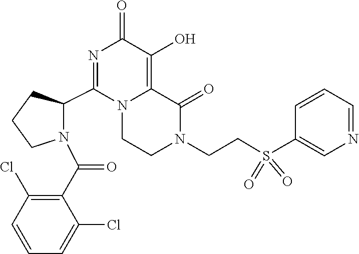 [SMILES: O=C1c2c(O)c(=O)nc([C@@H]3CCCN3C(=O)c3c(Cl)cccc3Cl)n2CCN1CCS(=O)(=O)c1cccnc1] | (S)-6-(1-(2,6- dichlorobenzoyl)pyrrolidin-2- yl)-9-hydroxy-2-(2-(pyridin-3- ylsulfonyl)ethyl)-3,4-dihydro- 2H-pyrazino[1,2-c]pyrimidine- 1,8-dione | 0.204 ± 0.028 | 0.023 ± 0.011 |
| SRI- 031021 | 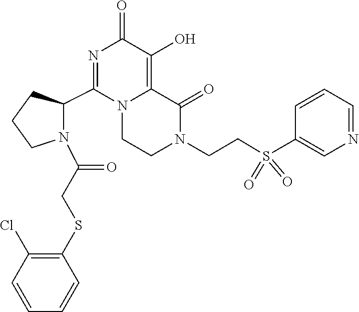 [SMILES: O=C1c2c(O)c(=O)nc([C@@H]3CCCN3C(=O)CSc3ccccc3Cl)n2CCN1CCS(=O)(=O)c1cccnc1] | (S)-6-(1-(2-((2- chlorophenyl)thio)acetyl) pyrrolidin-2-yl)-9- hydroxy-2-(2- (pyridin-3-ylsulfonyl) ethyl)-3,4-dihydro-2H- pyrazino[1,2- c]pyrimidine-1,8-dione | 0.193 ± 0.025 | 0.019 ± 0.010 |
| SRI- 031025 | 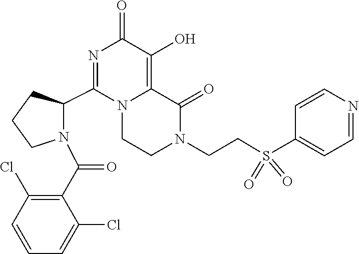 [SMILES: O=C1c2c(O)c(=O)nc([C@@H]3CCCN3C(=O)c3c(Cl)cccc3Cl)n2CCN1CCS(=O)(=O)c1ccncc1] | (S)-6-(1-(2,6- dichlorobenzoyl)pyrrolidin-2- yl)-9-hydroxy-2-(2-(pyridin-4- ylsulfonyl)ethyl)-3,4-dihydro- 2H-pyrazino[1,2-c]pyrimidine- 1,8-dione | 0.352 ± 0.161 | 0.036 ± 0.022 |
| SRI- 031029 | 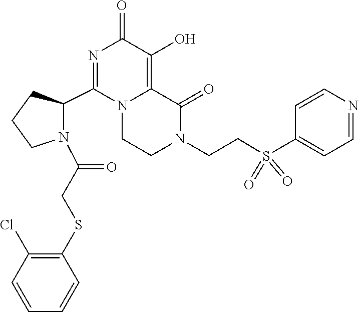 [SMILES: O=C1c2c(O)c(=O)nc([C@@H]3CCCN3C(=O)CSc3ccccc3Cl)n2CCN1CCS(=O)(=O)c1ccncc1] | (S)-6-(1-(2-((2- chlorophenyl)thio)acetyl) pyrrolidin-2-yl)-9- hydroxy-2-(2-(pyridin-4- ylsulfonyl)ethyl)-3,4- dihydro-2H-pyrazino[1,2- c]pyrimidine-1,8-dione | 0.144 ± 0.004 | −0.003 ± 0.002 |
| SRI- 031014 | 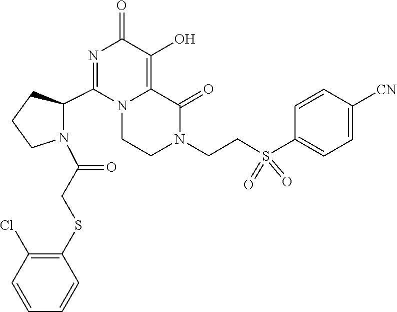 [SMILES: N#Cc1ccc(S(=O)(=O)CCN2CCn3c([C@@H]4CCCN4C(=O)CSc4ccccc4Cl)nc(=O)c(O)c3C2=O)cc1] | (S)-4-((2-(6-(1-(2-((2- chlorophenyl)thio)acetyl) pyrrolidin-2-yl)-9- hydroxy-1,8-dioxo- 1,3,4,8-tetrahydro-2H- pyrazino[1,2-c]pyrimidin-2- yl)ethyl)sulfonyl)benzonitrile | 0.234 ± 0.053 | 0.036 ± 0.022 |
| SRI- 031018 | 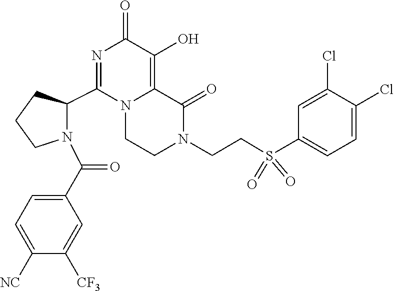 [SMILES: N#Cc1ccc(C(=O)N2CCC[C@H]2c2nc(=O)c(O)c3n2CCN(CCS(=O)(=O)c2ccc(Cl)c(Cl)c2)C3=O)cc1C(F)(F)F] | (S)-4-(2-(2-(2-((3,4- dichlorophenyl)sulfonyl)ethyl)- 9-hydroxy-1,8-dioxo-1,3,4,8- tetrahydro-2H-pyrazino[1,2- c]pyrimidin-6-yl)pyrrolidine-1- carbonyl)-2- (trifluoromethyl)benzonitrile | 0.221 ± 0.049 | 0.030 ± 0.020 |
| SRI- 031022 | 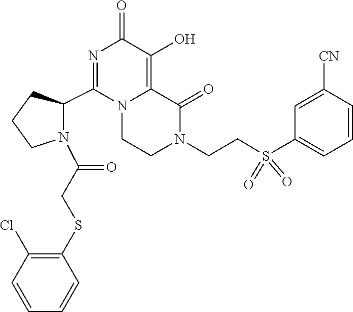 [SMILES: N#Cc1cccc(S(=O)(=O)CCN2CCn3c([C@@H]4CCCN4C(=O)CSc4ccccc4Cl)nc(=O)c(O)c3C2=O)c1] | (S)-3-((2-(6-(1-(2-((2- chlorophenyl)thio)acetyl) pyrrolidin-2-yl)-9-hydroxy- 1,8-dioxo-1,3,4,8- tetrahydro-2H- pyrazino[1,2-c]pyrimidin-2- yl)ethyl)sulfonyl)benzonitrile | 0.182 ± 0.019 | 0.014 ± 0.008 |
| SRI- 031026 | 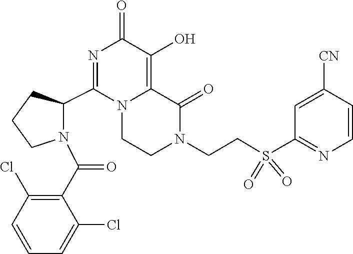 [SMILES: N#Cc1ccnc(S(=O)(=O)CCN2CCn3c([C@@H]4CCCN4C(=O)c4c(Cl)cccc4Cl)nc(=O)c(O)c3C2=O)c1] | (S)-2-((2-(6-(1-(2,6- dichlorobenzoyl)pyrrolidin-2- yl)-9-hydroxy-1,8-dioxo- 1,3,4,8-tetrahydro-2H- pyrazino[1,2-c]pyrimidin-2- yl)ethyl)sulfonyl)isonicotinonitr | 0.195 ± 0.021 | 0.015 ± 0.007 |
| SRI- 031030 | 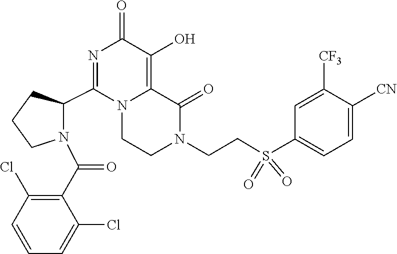 [SMILES: N#Cc1ccc(S(=O)(=O)CCN2CCn3c([C@@H]4CCCN4C(=O)c4c(Cl)cccc4Cl)nc(=O)c(O)c3C2=O)cc1C(F)(F)F] | (S)-4-((2-(6-(1-(2,6- dichlorobenzoyl)pyrrolidin-2- yl)-9-hydroxy-1,8-dioxo- 1,3,4,8-tetrahydro-2H- pyrazino[1,2-c]pyrimidin-2- yl)ethyl)sulfonyl)-2- | 0.438 ± 0.149 | 0.100 ± 0.005 |
| SRI- 031015 | 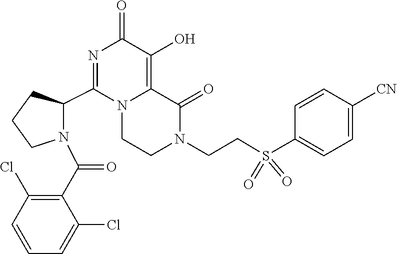 [SMILES: N#Cc1ccc(S(=O)(=O)CCN2CCn3c([C@@H]4CCCN4C(=O)c4c(Cl)cccc4Cl)nc(=O)c(O)c3C2=O)cc1] | (S)-4-((2-(6-(1-(2,6- dichlorobenzoyl)pyrrolidin-2- yl)-9-hydroxy-1,8-dioxo- 1,3,4,8-tetrahydro-2H- pyrazino[1,2-c]pyrimidin-2- yl)ethyl)sulfonyl)benzonitrile | 0.115 ± 0.006 | −0.013 ± 0.002 |
| SRI- 031019 | 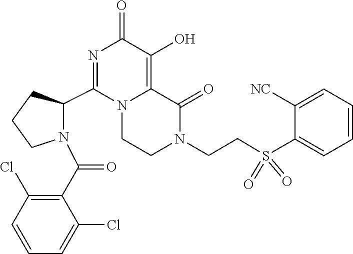 [SMILES: N#Cc1ccccc1S(=O)(=O)CCN1CCn2c([C@@H]3CCCN3C(=O)c3c(Cl)cccc3Cl)nc(=O)c(O)c2C1=O] | (S)-2-((2-(6-(1-(2,6- dichlorobenzoyl)pyrrolidin-2- yl)-9-hydroxy-1,8-dioxo- 1,3,4,8-tetrahydro-2H- pyrazino[1,2-c]pyrimidin-2- yl)ethyl)sulfonyl)benzonitrile | 1.124 ± 0.065 | 0.406 ± 0.027 |
| SRI- 031023 | 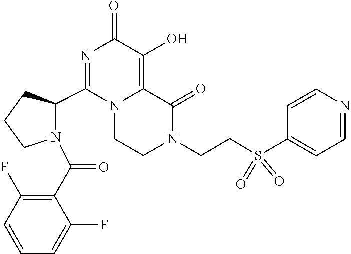 [SMILES: O=C1c2c(O)c(=O)nc([C@@H]3CCCN3C(=O)c3c(F)cccc3F)n2CCN1CCS(=O)(=O)c1ccncc1] | (S)-6-(1-(2,6- difluorobenzoyl)pyrrolidin-2- yl)-9-hydroxy-2-(2-(pyridin-4- ylsulfonyl)ethyl)-3,4-dihydro- 2H-pyrazino[1,2-c]pyrimidine- 1,8-dione | 0.824 ± 0.034 | 0.281 ± 0.014 |
| SRI- 031027 | 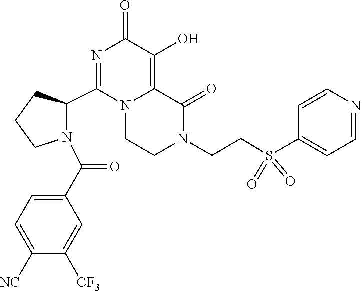 [SMILES: N#Cc1ccc(C(=O)N2CCC[C@H]2c2nc(=O)c(O)c3n2CCN(CCS(=O)(=O)c2ccncc2)C3=O)cc1C(F)(F)F] | (S)-4-(2-(9-hydroxy-1,8-dioxo- 2-(2-(pyridin-4- ylsulfonyl)ethyl)-1,3,4,8- tetrahydro-2H-pyrazino[1,2- c]pyrimidin-6-yl)pyrrolidine-1- carbonyl)-2- (trifluoromethyl)benzonitrile | 5.966 ± 1.074 | 2.053 ± 0.379 |

A list of additional exemplary compounds evaluated for potency and binding is shown in Table 4 below.

TABLE 4

| | | | Influenza |
|---|---|---|---|
| | | | Antiviral |
| | | | Activity by |
| | | | Cytopathic |
| | | PA | Effect (CPE) |
| | | Endonuclease | in MDCK |
| | | Binding by | Cells: EC₅₀ |
| No. | Structure | FP: Kᵢ (M) | (μM)* |
| SRI-0032119 | 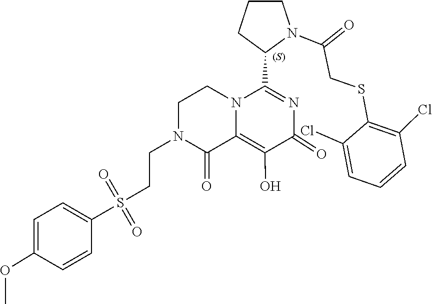 | 8E-08 | |
| SRI-0032118 | 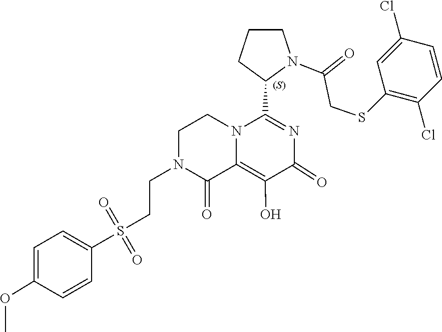 | 3.84E-08 | |
| SRI-0031030 | 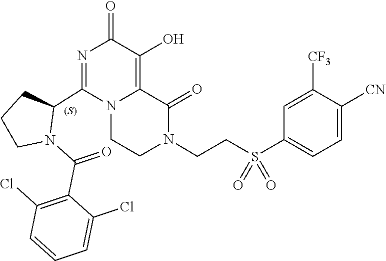 | 0.0000001 | |
| SRI-0031029 | 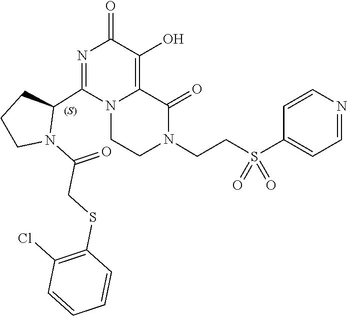 | <10.0E-09 | >40.0 |
| SRI-0031028 | 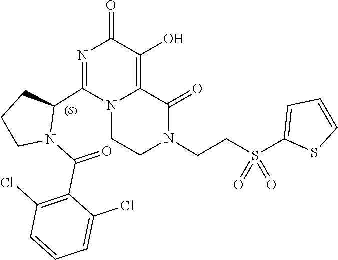 | 3.4E-08 | |
| SRI-0031027 | 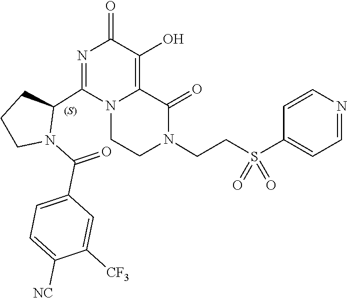 | 2.05E-06 | |
| SRI-0031026 | 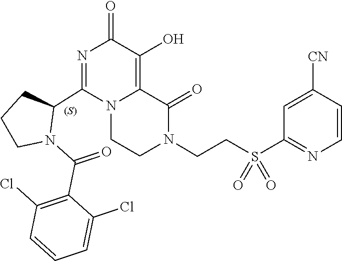 | 1.5E-08 | |
| SRI-0031025 | 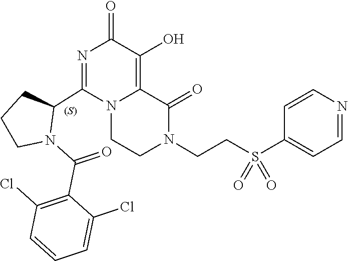 | 3.6E-08 | |
| SRI-0031024 | 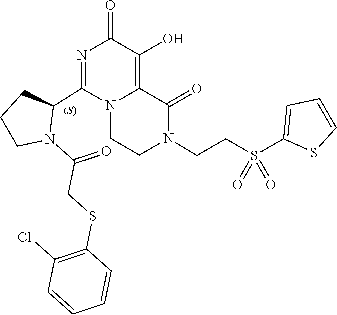 | <10.0E-09 | >40.0 |
| SRI-0031023 | 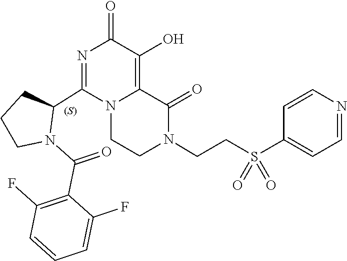 | 2.81E-07 | |
| SRI-0031022 | 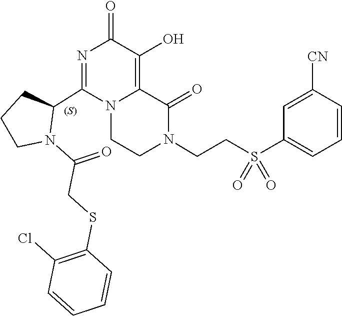 | 1.4E-08 | >40.0 |
| SRI-0031021 | 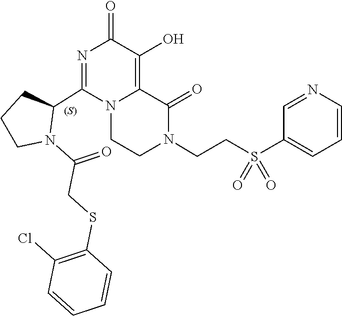 | 1.9E-08 | >40.0 |
| SRI-0031020 | 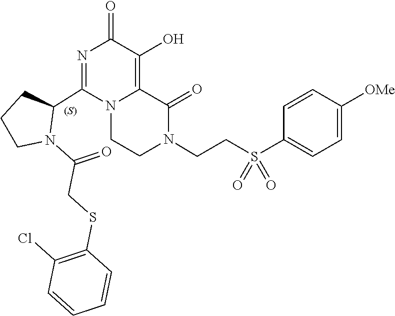 | <10.0E-09 | 39.3 |
| SRI-0031019 | 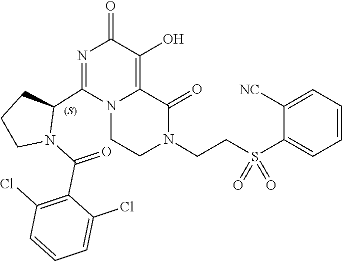 | 4.06E-07 | |
| SRI-0031018 | 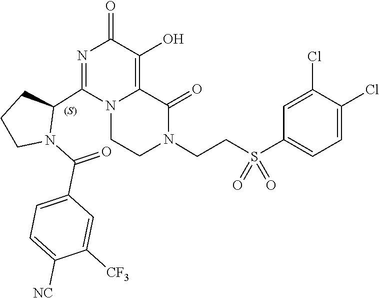 | 3E-08 | |
| SRI-0031017 | 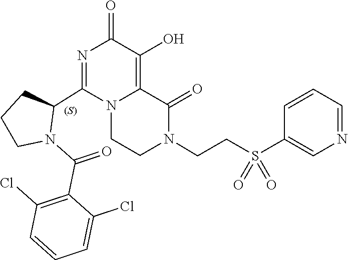 | 2.3E-08 | |
| SRI-0031016 | 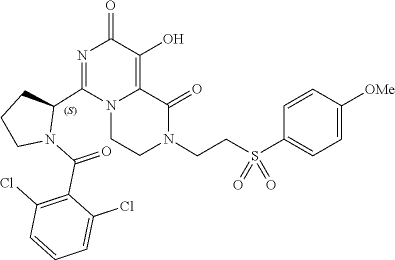 | <10.0E-09 | >40.0 |
| SRI-0031015 | 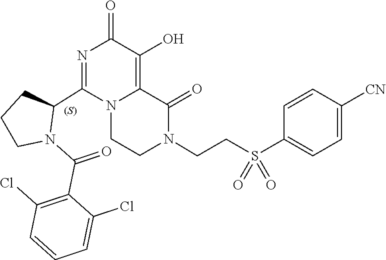 | <10.0E-09 | >40.0 |
| SRI-0031014 | 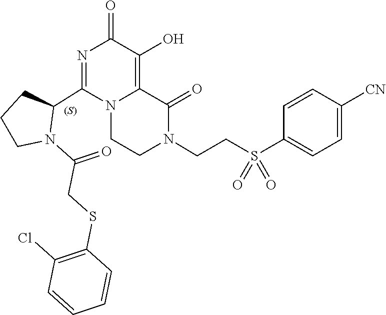 | 3.6E-08 | >50.0 |
| SRI-0031013 | 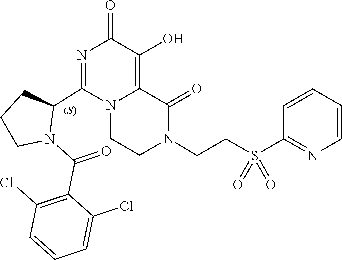 | 2.3E-08 | >50.0 |
| SRI-0030663 | 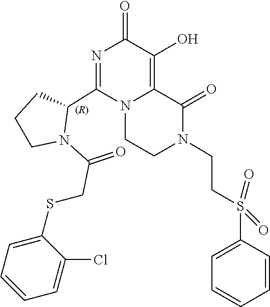 | 1.39E-07 | |
| SRI-0030662 | 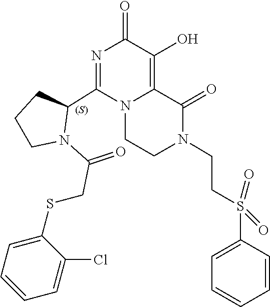 | 7.8E-08 | |
| SRI-0030652 | 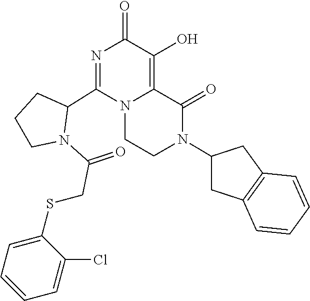 [SMILES: O=C1c2c(O)c(=O)nc(C3CCCN3C(=O)CSc3ccccc3Cl)n2CCN1C1Cc2ccccc2C1] | 3.97E-06 | |
| SRI-0030651 | 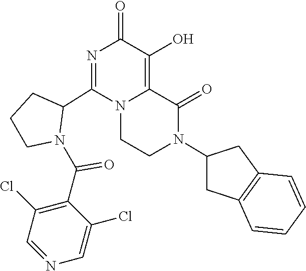 [SMILES: O=C1c2c(O)c(=O)nc(C3CCCN3C(=O)c3c(Cl)cncc3Cl)n2CCN1C1Cc2ccccc2C1] | 5.53E-06 | |
| SRI-0030650 | 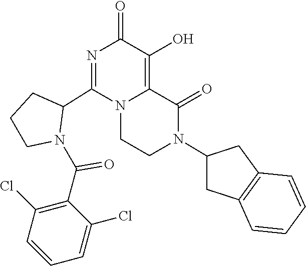 [SMILES: O=C1c2c(O)c(=O)nc(C3CCCN3C(=O)c3c(Cl)cccc3Cl)n2CCN1C1Cc2ccccc2C1] | 3.39E-06 | |
| SRI-0030649 | 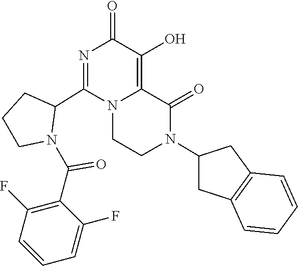 [SMILES: O=C1c2c(O)c(=O)nc(C3CCCN3C(=O)c3c(F)cccc3F)n2CCN1C1Cc2ccccc2C1] | 1.79E-06 | |
| SRI-0030512 | 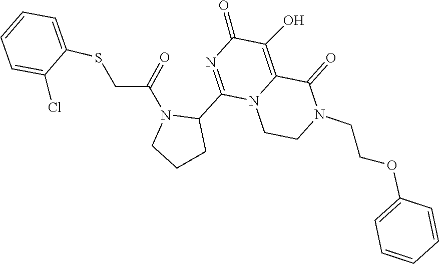 [SMILES: O=C1c2c(O)c(=O)nc(C3CCCN3C(=O)CSc3ccccc3Cl)n2CCN1CCOc1ccccc1] | 5.49E-07 | |
| SRI-0030511 | 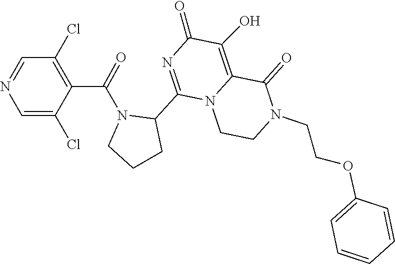 [SMILES: O=C1c2c(O)c(=O)nc(C3CCCN3C(=O)c3c(Cl)cncc3Cl)n2CCN1CCOc1ccccc1] | 2.22E-07 | |
| SRI-0030423 | 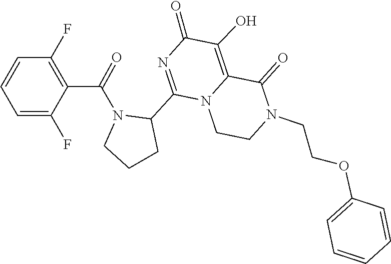 [SMILES: O=C1c2c(O)c(=O)nc(C3CCCN3C(=O)c3c(F)cccc3F)n2CCN1CCOc1ccccc1] | 2.37E-07 | |
| SRI-0030407 | 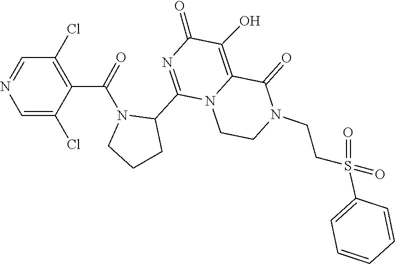 [SMILES: O=C1c2c(O)c(=O)nc(C3CCCN3C(=O)c3c(Cl)cncc3Cl)n2CCN1CCS(=O)(=O)c1ccccc1] | 3.05E-07 | |
| SRI-0030406 | 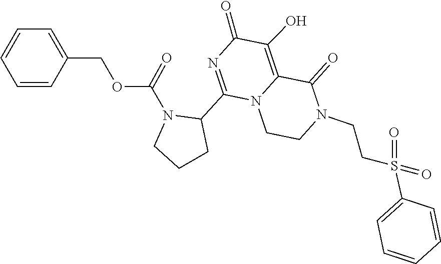 [SMILES: O=C1c2c(O)c(=O)nc(C3CCCN3C(=O)OCc3ccccc3)n2CCN1CCS(=O)(=O)c1ccccc1] | 1.75E-07 | |
| SRI-0030405 | 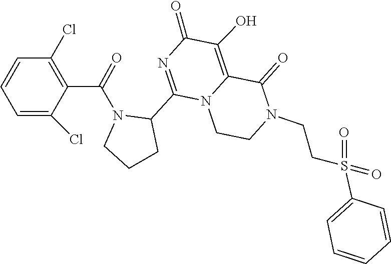 [SMILES: O=C1c2c(O)c(=O)nc(C3CCCN3C(=O)c3c(Cl)cccc3Cl)n2CCN1CCS(=O)(=O)c1ccccc1] | 7.9E-08 | |
| SRI-0030405 | 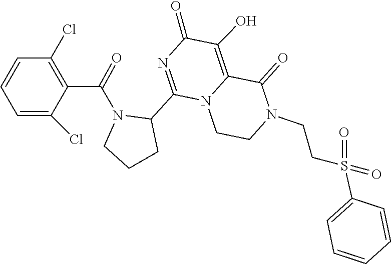 [SMILES: O=C1c2c(O)c(=O)nc(C3CCCN3C(=O)c3c(Cl)cccc3Cl)n2CCN1CCS(=O)(=O)c1ccccc1] | 8.2E-08 | |
| SRI-0030396 | 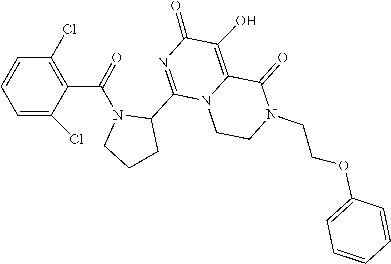 [SMILES: O=C1c2c(O)c(=O)nc(C3CCCN3C(=O)c3c(Cl)cccc3Cl)n2CCN1CCOc1ccccc1] | 4.48E-07 | >50.0 |
| SRI-0030395 | 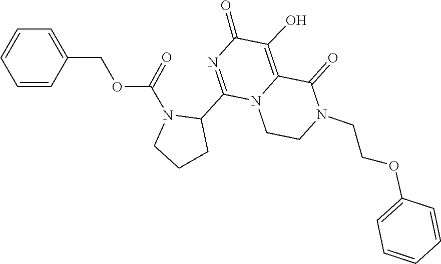 [SMILES: O=C1c2c(O)c(=O)nc(C3CCCN3C(=O)OCc3ccccc3)n2CCN1CCOc1ccccc1] | 1.22E-06 | >50.0 |
| SRI-0030330 | 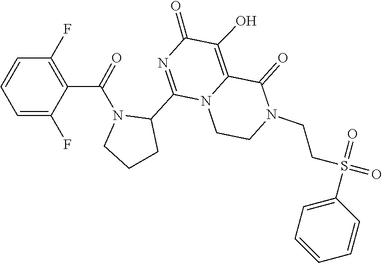 [SMILES: O=C1c2c(O)c(=O)nc(C3CCCN3C(=O)c3c(F)cccc3F)n2CCN1CCS(=O)(=O)c1ccccc1] | 6.7E-08 | |
| SRI-0030295 | 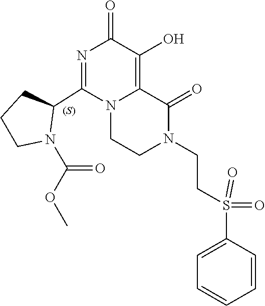 | 6.89E-07 | |
| SRI-0030292 | 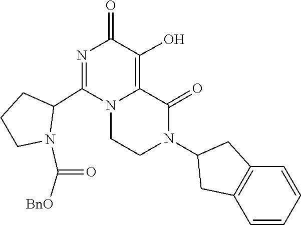 [SMILES: O=C1c2c(O)c(=O)nc(C3CCCN3C(=O)OCc3ccccc3)n2CCN1C1Cc2ccccc2C1] | 1.98E-06 | |
| SRI-0030290 | 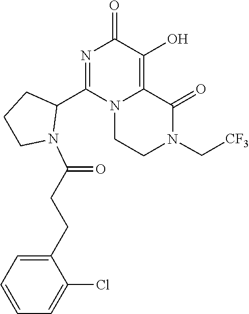 [SMILES: O=C1c2c(O)c(=O)nc(C3CCCN3C(=O)CCc3ccccc3Cl)n2CCN1CC(F)(F)F] | 6.03E-06 | |
| SRI-0030289 | 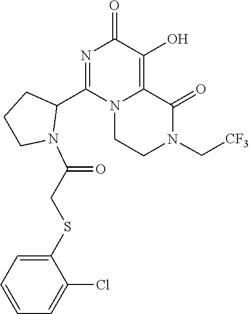 [SMILES: O=C1c2c(O)c(=O)nc(C3CCCN3C(=O)CSc3ccccc3Cl)n2CCN1CC(F)(F)F] | 4.23E-06 | |
| SRI-0030288 | 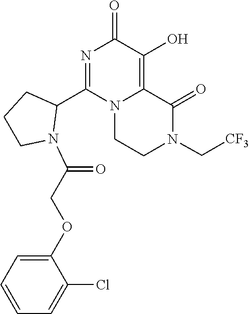 [SMILES: O=C1c2c(O)c(=O)nc(C3CCCN3C(=O)COc3ccccc3Cl)n2CCN1CC(F)(F)F] | 3.23E-06 | |
| SRI-0030287 | 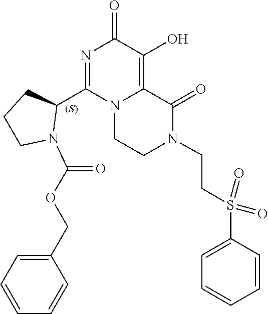 | 1.12E-07 | |
| SRI-0030275 | 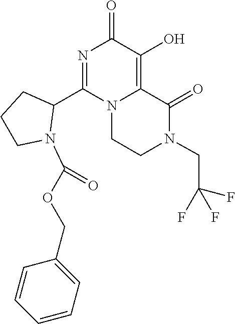 [SMILES: O=C1c2c(O)c(=O)nc(C3CCCN3C(=O)OCc3ccccc3)n2CCN1CC(F)(F)F] | 3.97E-06 | |
| SRI-0030274 | 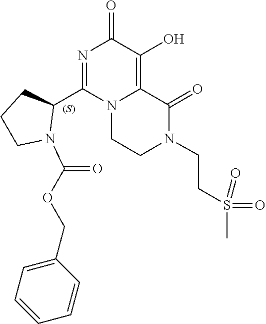 | 4.71E-06 | |
| SRI-0030273 | 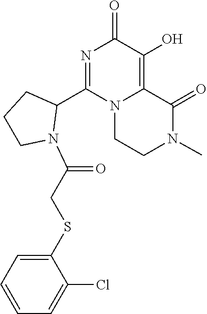 [SMILES: CN1CCn2c(C3CCCN3C(=O)CSc3ccccc3Cl)nc(=O)c(O)c2C1=O] | 1.22E-06 | |
| SRI-0030272 | 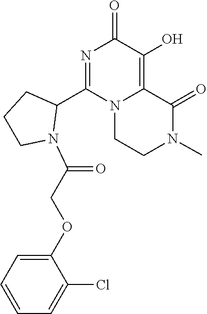 [SMILES: CN1CCn2c(C3CCCN3C(=O)COc3ccccc3Cl)nc(=O)c(O)c2C1=O] | 3.27E-06 | |
| SRI-0030272 | 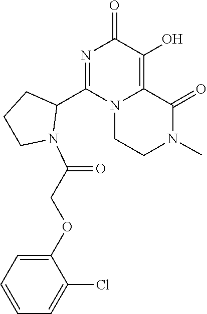 [SMILES: CN1CCn2c(C3CCCN3C(=O)COc3ccccc3Cl)nc(=O)c(O)c2C1=O] | 4.16E-06 | |
| SRI-0030269 | 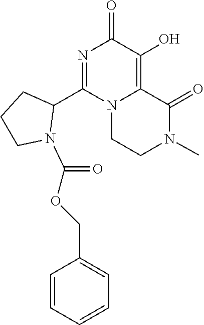 [SMILES: CN1CCn2c(C3CCCN3C(=O)OCc3ccccc3)nc(=O)c(O)c2C1=O] | 3.43E-06 | |

*Virus Type: Influenza A, Virus Strain: PR8, M.O.I.: 0.0005

The references listed herein are also part of the application and are incorporated by reference in their entirety as if fully set forth herein.

What is claimed is:

1. A compound having a structure represented by a formula: 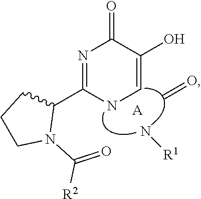
wherein:
A is a 6-7 membered heterocycle;
R¹ is C1-C3 alkyl, C1-C3 alkoxy, C1-C3 haloalkyl, C1-C3 alkylsulfonyl, or a 9- to 10-membered cycloaryl, wherein R¹ can further be independently substituted with one or more R^{x} groups;
R^{x} is sulfonyl, oxo, C1-C2 haloalkyl, 5- to 6-membered aryl, or 5- to 6-membered heteroaryl, wherein R^{x} can independently further be substituted with one or more R^{a} groups;
R^{a} is C1-C2 alkyl, 5- to 6-membered aryl, 5- to 6-membered heteroaryl, or C1-C2 haloalkyl, wherein R^{a} can independently be substituted with one or more R^{a1} groups;
R^{a1} is halo, C1-C2 alkoxy, cyano, or C1-C2 haloalkyl;
R² is C1-C2 alkyl, 5- to 6-membered aryl, oxo, 5- to 6-membered heteroaryl, or C1-C2 alkoxy, wherein R² can further be independently substituted with one or more R^{y} groups;
R^{y} is halo, oxo, C1-C2 alkyl, sulfidyl, cyano, C1-C2 haloalkyl, or 5- to 6-membered aryl, wherein R^{y} can independently further be substituted with one or more R^{b} groups;
R^{b} is halo or 5- to 6-membered aryl;
R^{b1} is halo; and
wherein the wavy line indicates either R or S enantiomer at that bond,
or a pharmaceutically acceptable salt or hydrate thereof.

2. The compound of claim 1, wherein the compound has a structure represented by a formula: 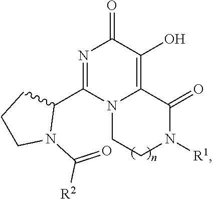
wherein:
n is 1 or 2;
R¹ is C1-C3 alkyl, C1-C3 haloalkyl, —(C1-C3 alkyl)OR¹⁰, —(C1-C3 alkyl)SO₂R¹⁰, or Cy¹;
R¹⁰ is C1-C2 alkyl or Ar¹;
Ar¹ is a 5- to 6-membered aryl or a 5- to 6-membered heteroaryl, and is substituted with 0, 1, 2, or 3 groups independently selected from halogen, —CN, C1-C2 alkyl, C1-C2 haloalkyl, and —C1-C2 alkoxy;
Cy¹ is an unsubstituted 9- to 10-membered cycloalkyl group;
R² is C1-C2 alkyl, —(C1-C2 alkyl)Ar², —O(C1-C2 alkyl), —O(C1-C2 alkyl)Ar², —(C1-C2 alkyl)OAr², —S(C1-C2 alkyl), —S(C1-C2 alkyl)Ar², —(C1-C2 alkyl)SAr², or Ar²; and
Ar² is a 5- to 6-membered aryl or a 5- to 6-membered heteroaryl, and is substituted with 0, 1, 2, or 3 groups independently selected from halogen, —CN, C1-C2 alkyl, and C1-C2 haloalkyl,
or a pharmaceutically acceptable salt thereof.

3. The compound of claim 2, wherein n is 1.

4. The compound of claim 2, wherein n is 2.

5. The compound of claim 2, wherein R¹ is a structure selected from:

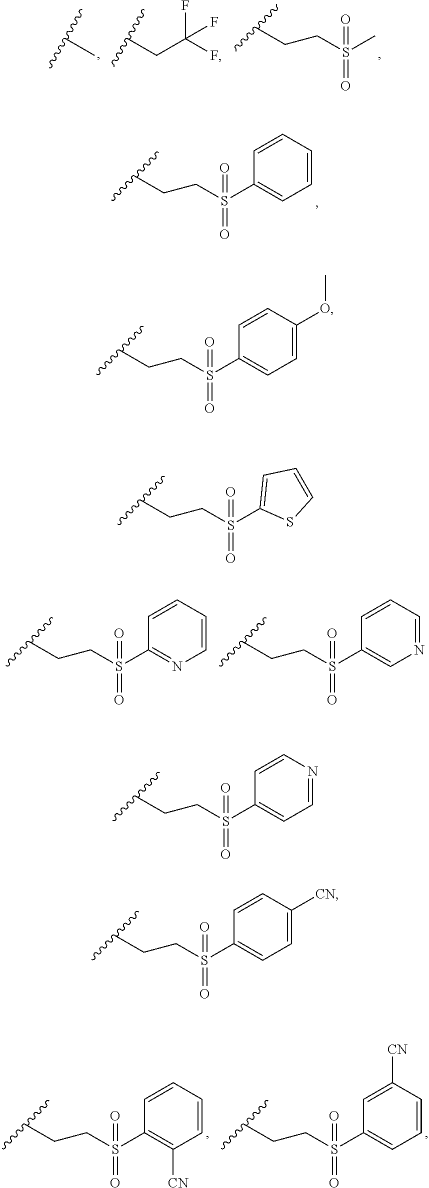

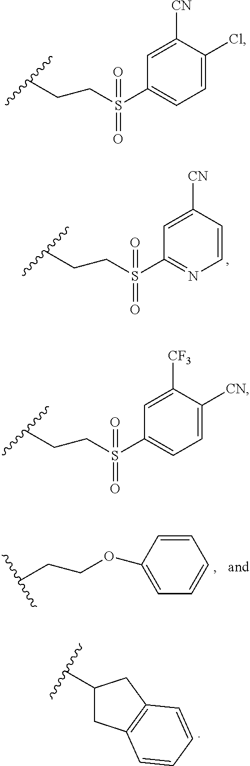 [SMILES: *CCS(=O)(=O)c1ccc(Cl)c(C#N)c1 ; *CCS(=O)(=O)c1cc(C#N)ccn1 ; *CCS(=O)(=O)c1ccc(C#N)c(C(F)(F)F)c1 ; *CCOc1ccccc1 ; *C1Cc2ccccc2C1]

6. The compound of claim 2, wherein R² is a structure selected from:

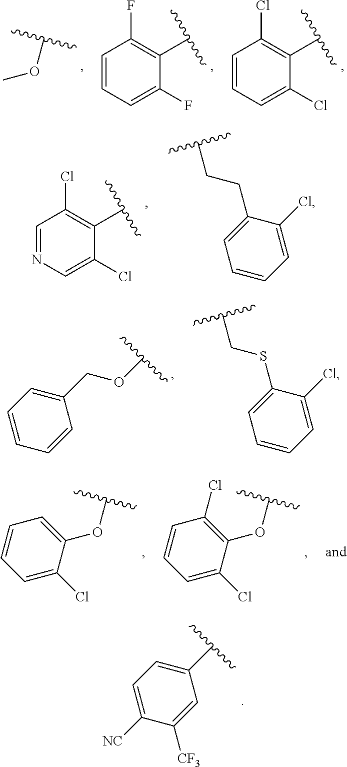

7. The compound of claim 2, wherein the compound has a structure represented by a formula:

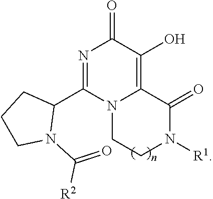

8. The compound of claim 2, wherein the compound has a structure represented by a formula:

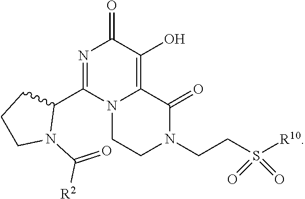

9. The compound of claim 8, wherein R¹⁰ is a structure selected from

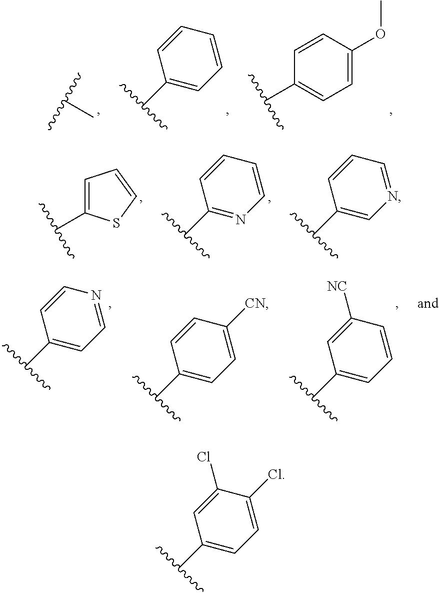

10. The compound of claim 8, wherein R² is a structure selected from:

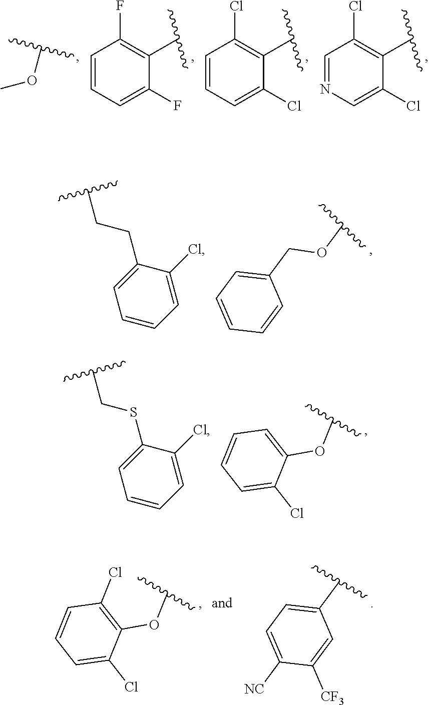

11. The compound of claim 2, wherein the compound has a structure: 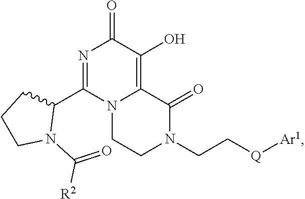
wherein:
Q is O or SO₂.

12. The compound of claim 11, wherein the compound has a structure: 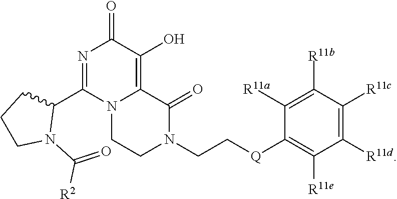
wherein:
each of R^{11a}, R^{11b}, R^{11c}, R^{11d}, and R^{11e} is independently selected from hydrogen, halogen, —CN, C1-C2 alkyl, C1-C2 haloalkyl, and —C1-C2 alkoxy, provided that at least two of R^{11a}, R^{11b}, R^{11c}, R^{11d} and R^{11e} are hydrogen.

13. The compound of claim 2, wherein the compound is selected from:

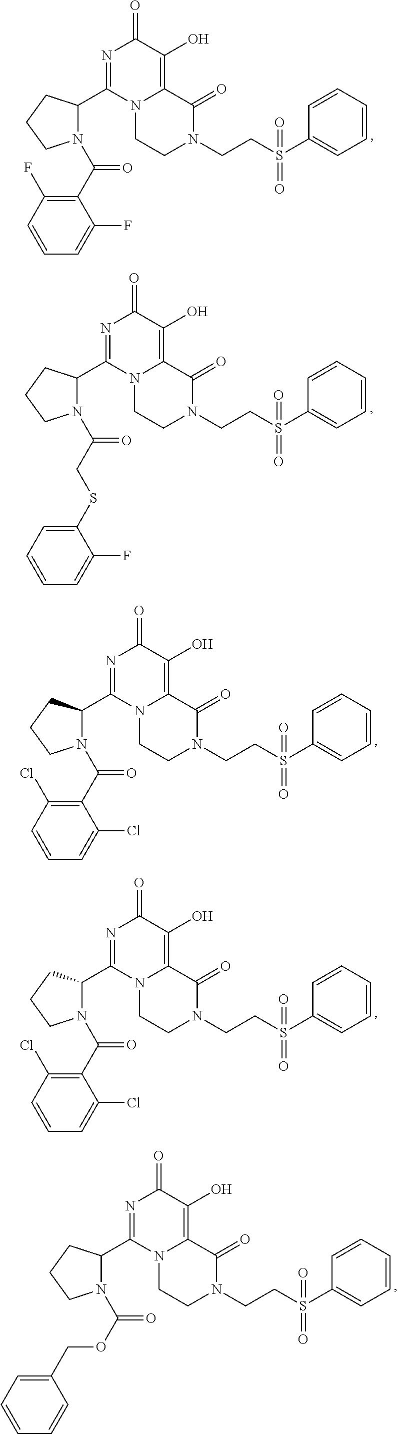 [SMILES: O=C1c2c(O)c(=O)nc(C3CCCN3C(=O)c3c(F)cccc3F)n2CCN1CCS(=O)(=O)c1ccccc1 ; O=C1c2c(O)c(=O)nc(C3CCCN3C(=O)CSc3ccccc3F)n2CCN1CCS(=O)(=O)c1ccccc1 ; O=C1c2c(O)c(=O)nc([C@@H]3CCCN3C(=O)c3c(Cl)cccc3Cl)n2CCN1CCS(=O)(=O)c1ccccc1 ; O=C1c2c(O)c(=O)nc([C@H]3CCCN3C(=O)c3c(Cl)cccc3Cl)n2CCN1CCS(=O)(=O)c1ccccc1 ; O=C1c2c(O)c(=O)nc(C3CCCN3C(=O)OCc3ccccc3)n2CCN1CCS(=O)(=O)c1ccccc1]

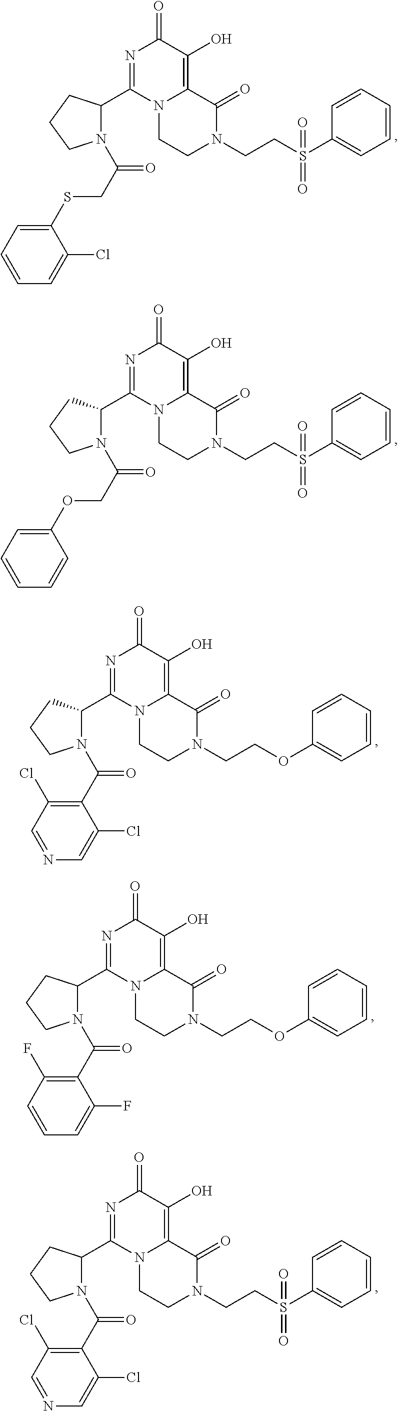 [SMILES: O=C1c2c(O)c(=O)nc(C3CCCN3C(=O)CSc3ccccc3Cl)n2CCN1CCS(=O)(=O)c1ccccc1 ; O=C1c2c(O)c(=O)nc([C@H]3CCCN3C(=O)COc3ccccc3)n2CCN1CCS(=O)(=O)c1ccccc1 ; O=C1c2c(O)c(=O)nc([C@H]3CCCN3C(=O)c3c(Cl)cncc3Cl)n2CCN1CCOc1ccccc1 ; O=C1c2c(O)c(=O)nc(C3CCCN3C(=O)c3c(F)cccc3F)n2CCN1CCOc1ccccc1 ; O=C1c2c(O)c(=O)nc(C3CCCN3C(=O)c3c(Cl)cncc3Cl)n2CCN1CCS(=O)(=O)c1ccccc1]

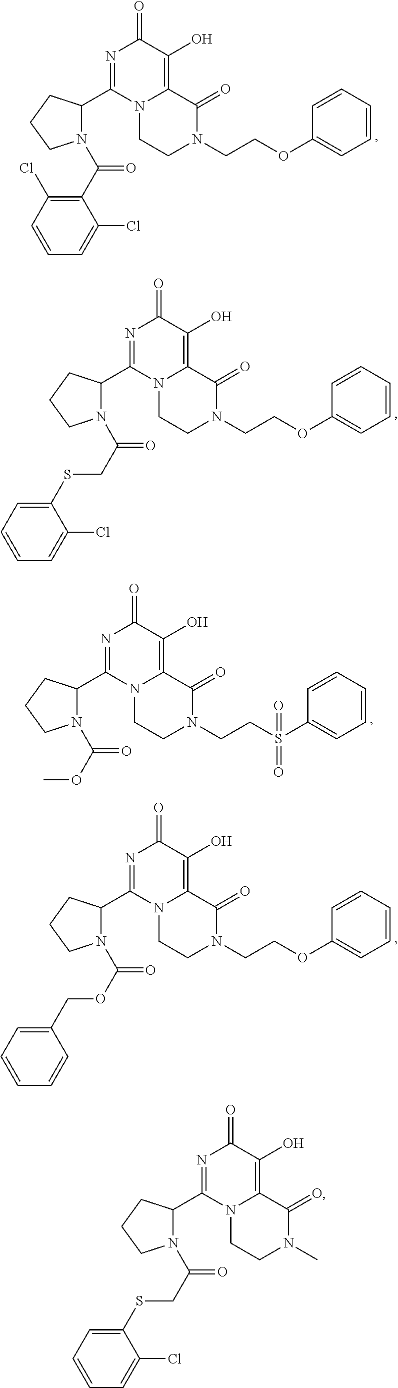 [SMILES: O=C1c2c(O)c(=O)nc(C3CCCN3C(=O)c3c(Cl)cccc3Cl)n2CCN1CCOc1ccccc1 ; O=C1c2c(O)c(=O)nc(C3CCCN3C(=O)CSc3ccccc3Cl)n2CCN1CCOc1ccccc1 ; COC(=O)N1CCCC1c1nc(=O)c(O)c2n1CCN(CCS(=O)(=O)c1ccccc1)C2=O ; O=C1c2c(O)c(=O)nc(C3CCCN3C(=O)OCc3ccccc3)n2CCN1CCOc1ccccc1 ; CN1CCn2c(C3CCCN3C(=O)CSc3ccccc3Cl)nc(=O)c(O)c2C1=O]

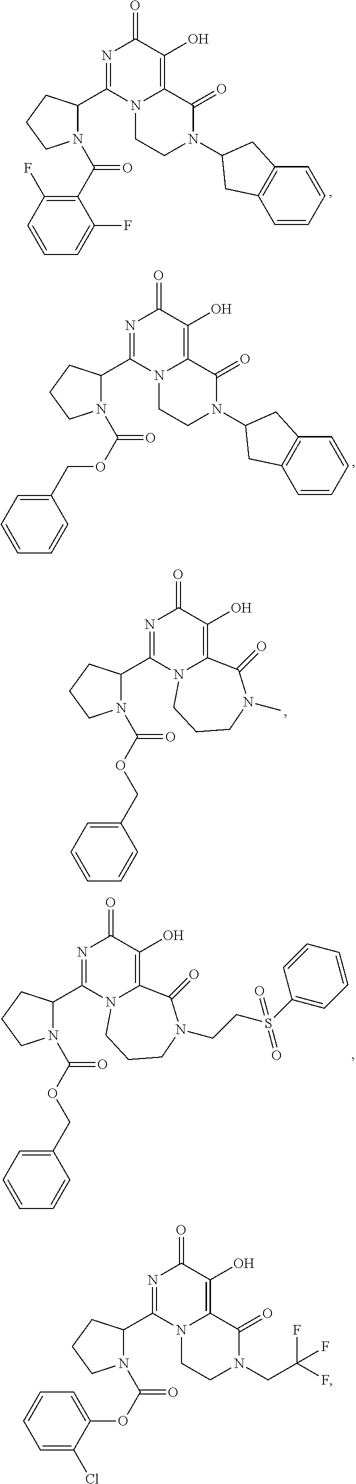

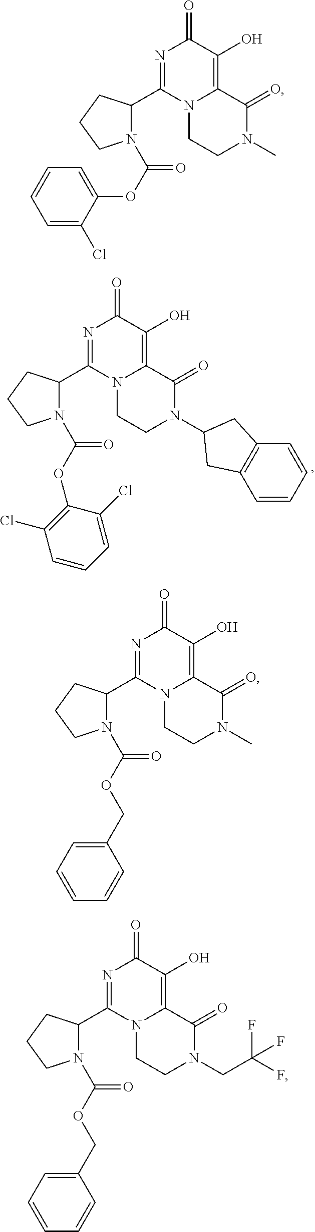

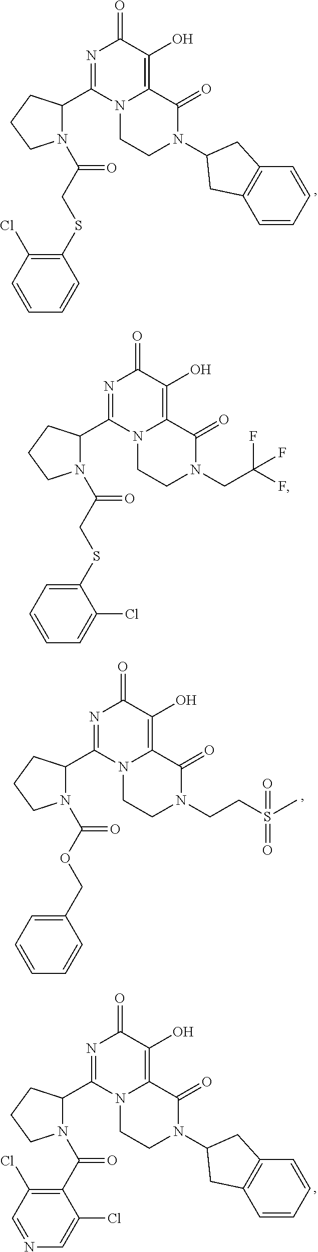 [SMILES: O=C1c2c(O)c(=O)nc(C3CCCN3C(=O)CSc3ccccc3Cl)n2CCN1C1Cc2ccccc2C1 ; O=C1c2c(O)c(=O)nc(C3CCCN3C(=O)CSc3ccccc3Cl)n2CCN1CC(F)(F)F ; CS(=O)(=O)CCN1CCn2c(C3CCCN3C(=O)OCc3ccccc3)nc(=O)c(O)c2C1=O ; O=C1c2c(O)c(=O)nc(C3CCCN3C(=O)c3c(Cl)cncc3Cl)n2CCN1C1Cc2ccccc2C1]

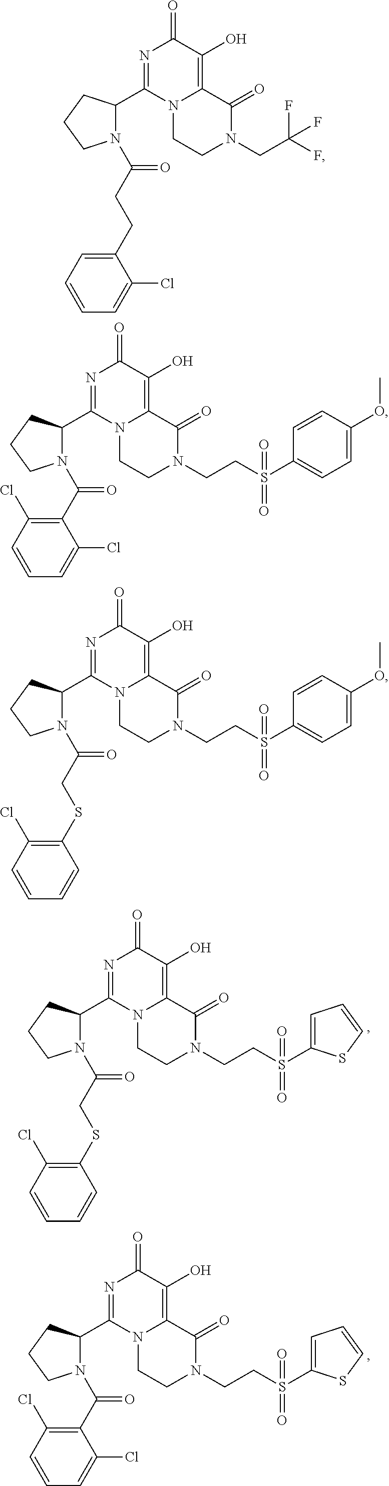

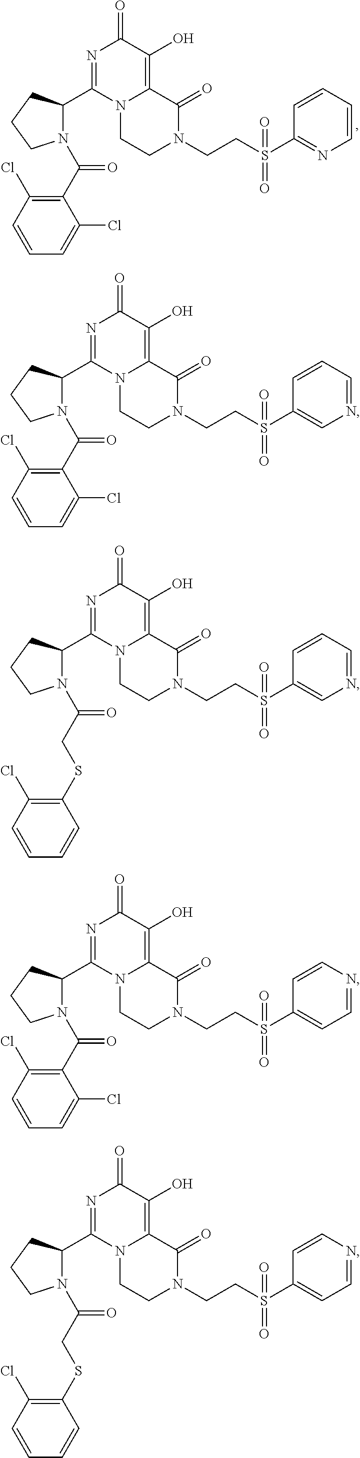 [SMILES: O=C1c2c(O)c(=O)nc([C@@H]3CCCN3C(=O)c3c(Cl)cccc3Cl)n2CCN1CCS(=O)(=O)c1ccccn1 ; O=C1c2c(O)c(=O)nc([C@@H]3CCCN3C(=O)c3c(Cl)cccc3Cl)n2CCN1CCS(=O)(=O)c1cccnc1 ; O=C1c2c(O)c(=O)nc([C@@H]3CCCN3C(=O)CSc3ccccc3Cl)n2CCN1CCS(=O)(=O)c1cccnc1 ; O=C1c2c(O)c(=O)nc([C@@H]3CCCN3C(=O)c3c(Cl)cccc3Cl)n2CCN1CCS(=O)(=O)c1ccncc1 ; O=C1c2c(O)c(=O)nc([C@@H]3CCCN3C(=O)CSc3ccccc3Cl)n2CCN1CCS(=O)(=O)c1ccncc1]

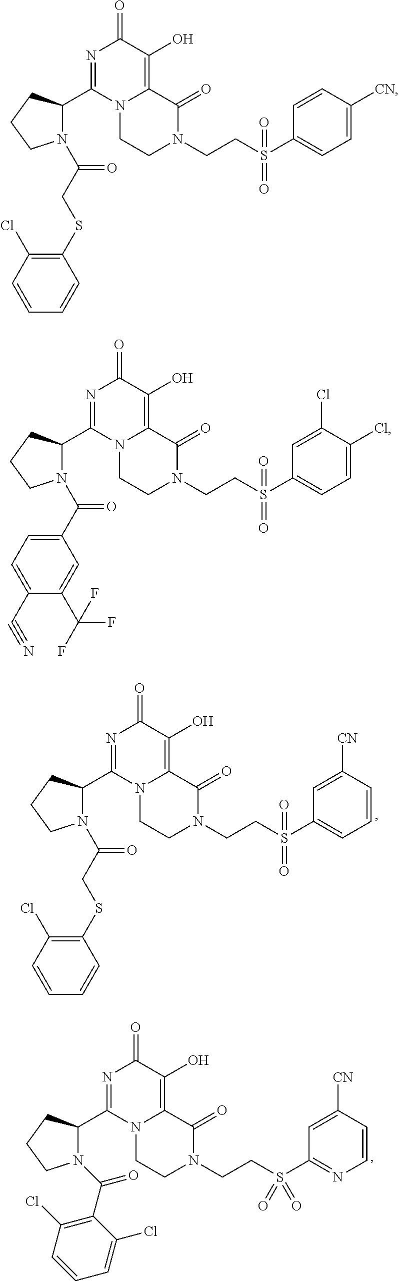 [SMILES: N#Cc1ccc(S(=O)(=O)CCN2CCn3c([C@@H]4CCCN4C(=O)CSc4ccccc4Cl)nc(=O)c(O)c3C2=O)cc1 ; N#Cc1ccc(C(=O)N2CCC[C@H]2c2nc(=O)c(O)c3n2CCN(CCS(=O)(=O)c2ccc(Cl)c(Cl)c2)C3=O)cc1C(F)(F)F ; N#Cc1cccc(S(=O)(=O)CCN2CCn3c([C@@H]4CCCN4C(=O)CSc4ccccc4Cl)nc(=O)c(O)c3C2=O)c1 ; N#Cc1ccnc(S(=O)(=O)CCN2CCn3c([C@@H]4CCCN4C(=O)c4c(Cl)cccc4Cl)nc(=O)c(O)c3C2=O)c1]

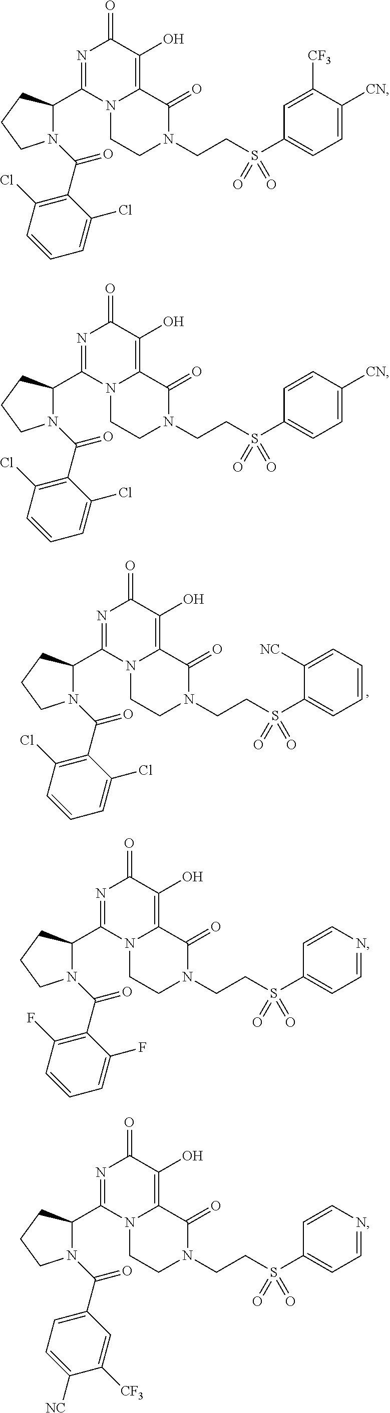

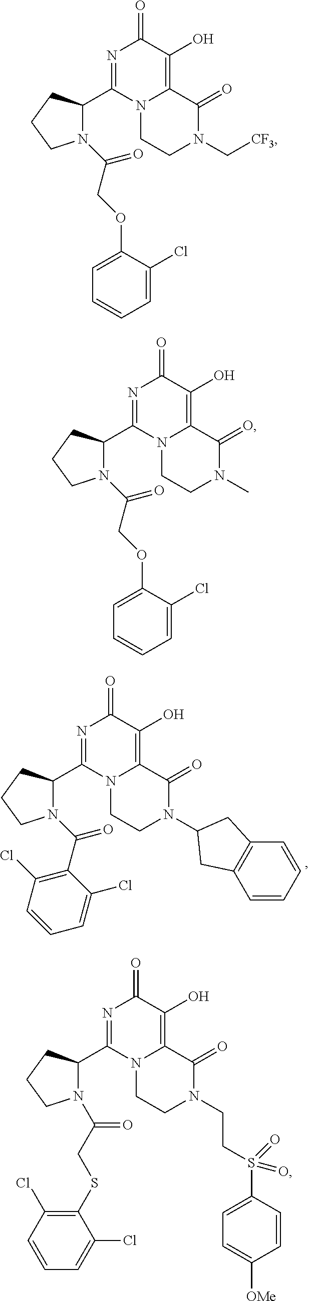

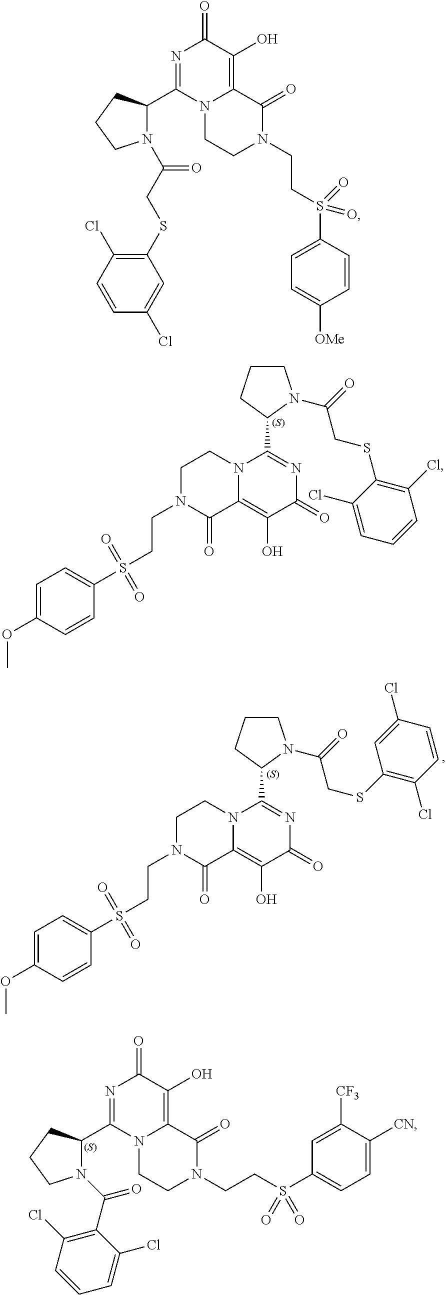

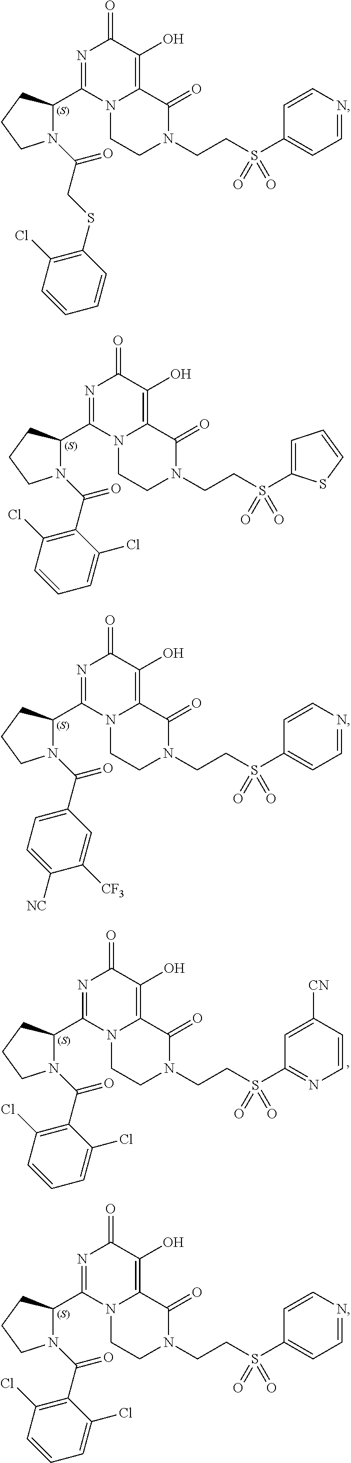

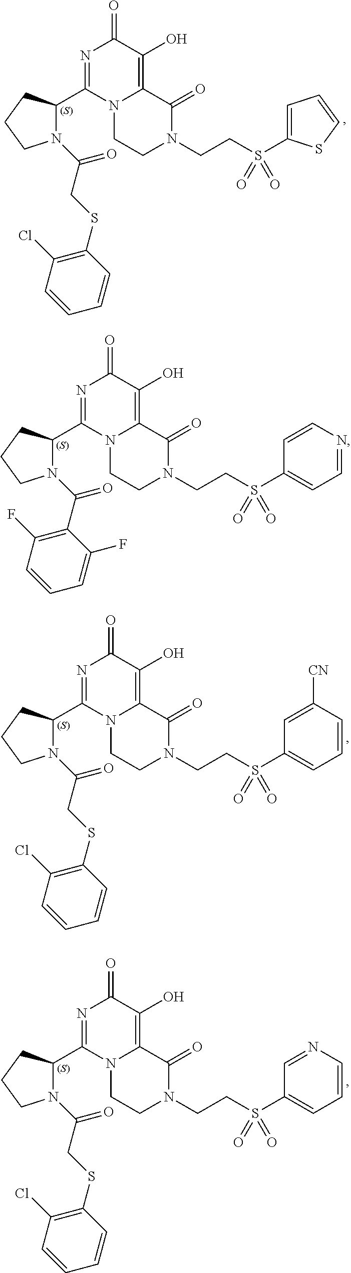

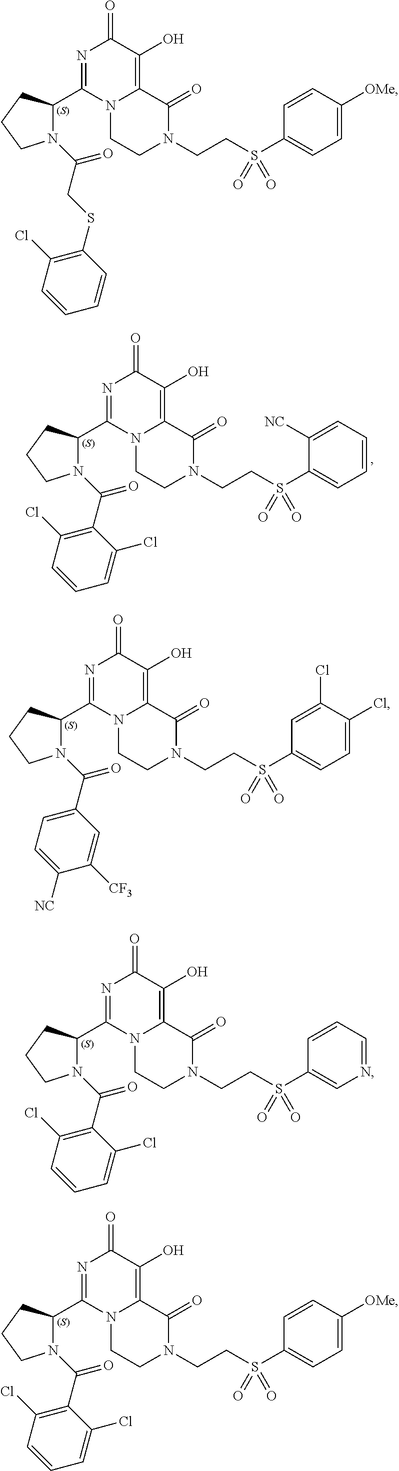

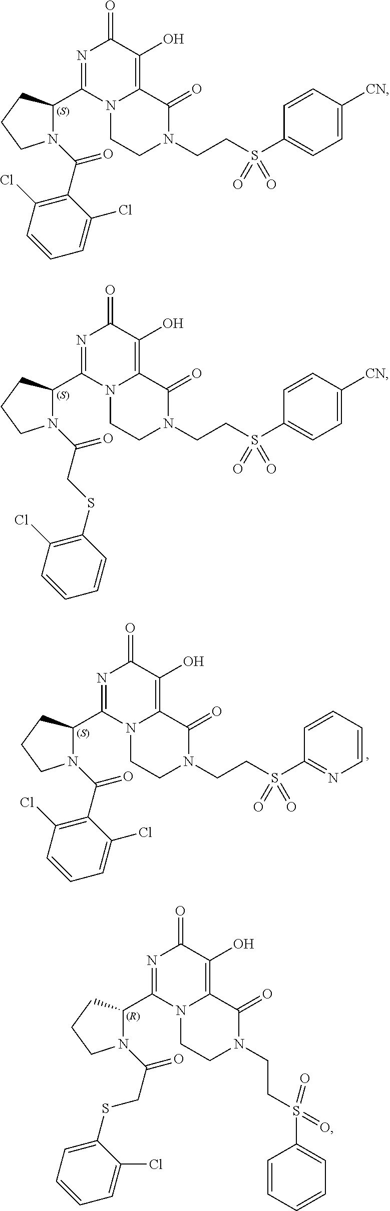

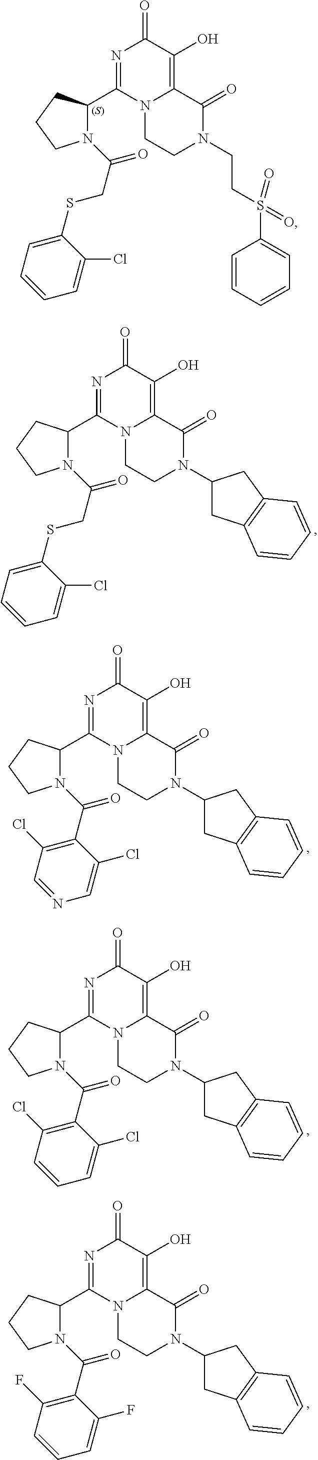

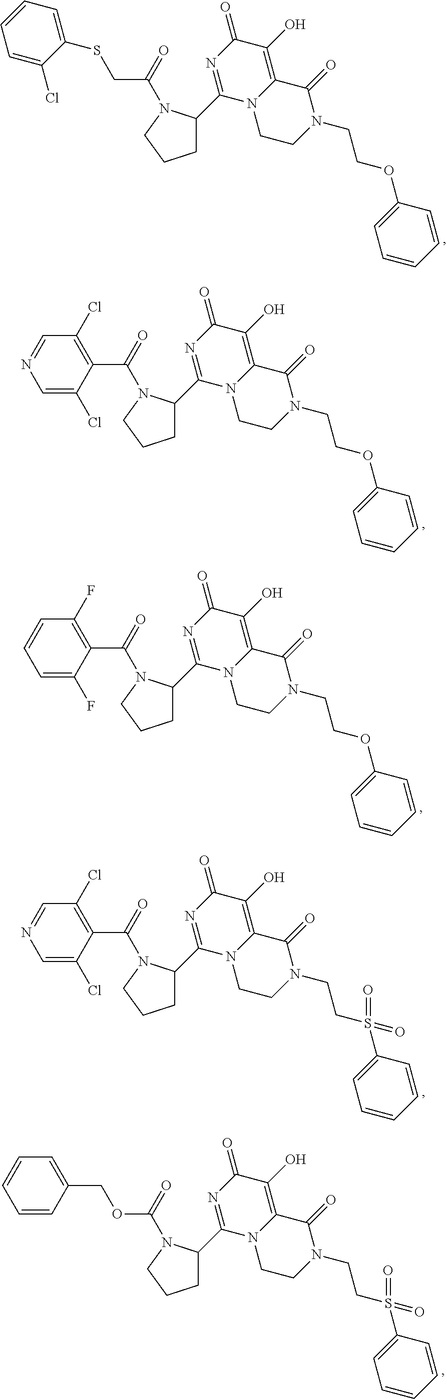 [SMILES: O=C1c2c(O)c(=O)nc(C3CCCN3C(=O)CSc3ccccc3Cl)n2CCN1CCOc1ccccc1 ; O=C1c2c(O)c(=O)nc(C3CCCN3C(=O)c3c(Cl)cncc3Cl)n2CCN1CCOc1ccccc1 ; O=C1c2c(O)c(=O)nc(C3CCCN3C(=O)c3c(F)cccc3F)n2CCN1CCOc1ccccc1 ; O=C1c2c(O)c(=O)nc(C3CCCN3C(=O)c3c(Cl)cncc3Cl)n2CCN1CCS(=O)(=O)c1ccccc1 ; O=C1c2c(O)c(=O)nc(C3CCCN3C(=O)OCc3ccccc3)n2CCN1CCS(=O)(=O)c1ccccc1]

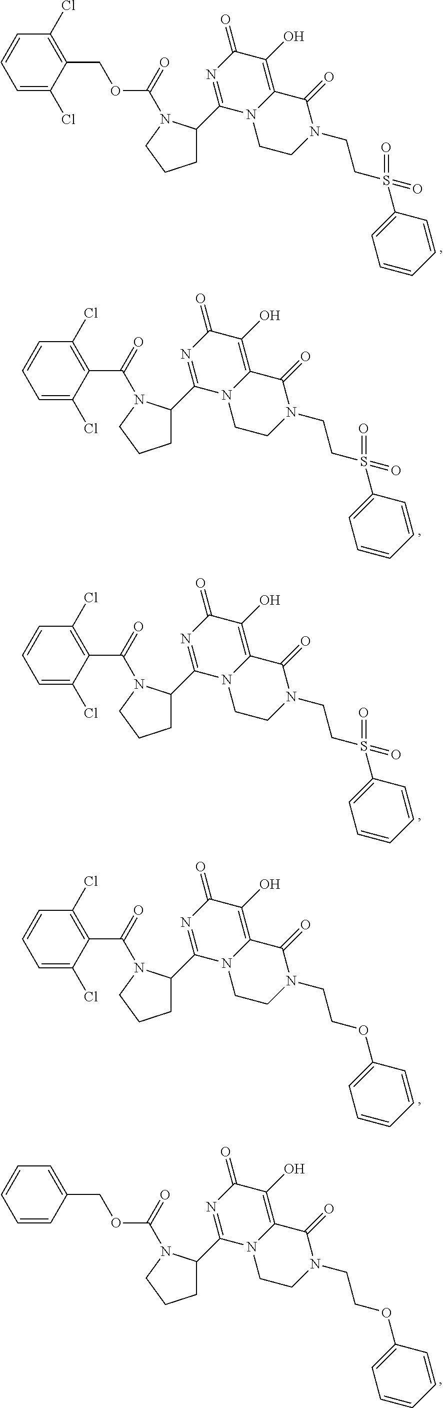 [SMILES: O=C1c2c(O)c(=O)nc(C3CCCN3C(=O)OCc3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCS(=O)(=O)c1ccccc1 ; O=C1c2c(O)c(=O)nc(C3CCCN3C(=O)c3c(Cl)cccc3Cl)n2CCN1CCOc1ccccc1 ; O=C1c2c(O)c(=O)nc(C3CCCN3C(=O)OCc3ccccc3)n2CCN1CCOc1ccccc1]

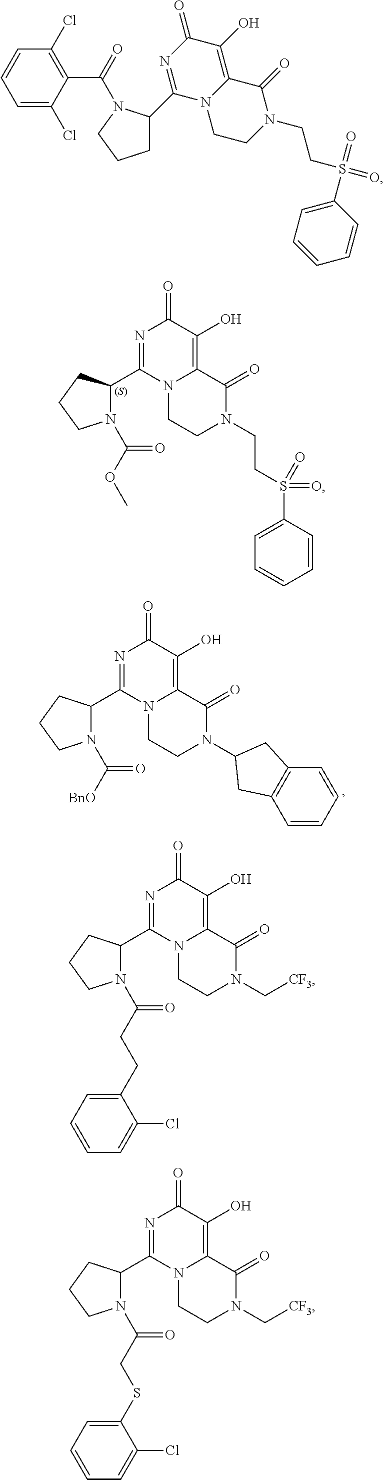

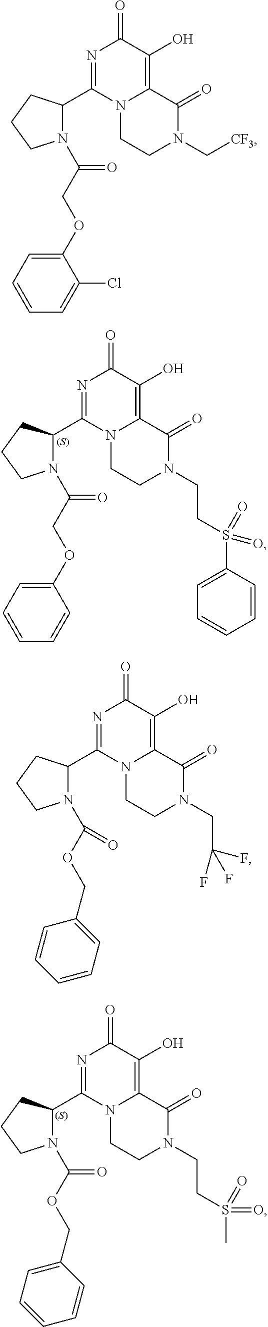

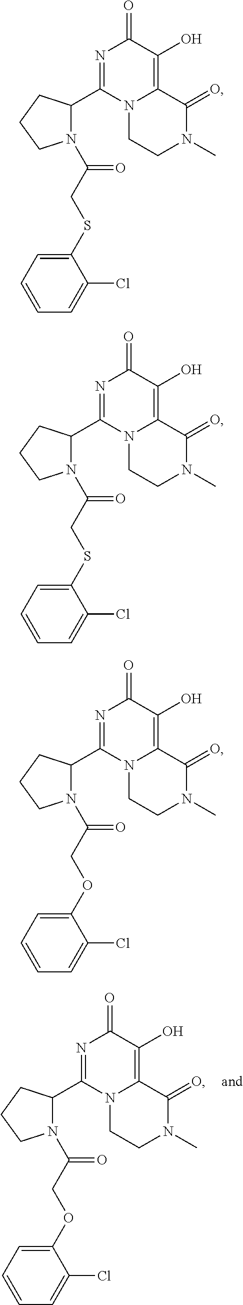 [SMILES: CN1CCn2c(C3CCCN3C(=O)CSc3ccccc3Cl)nc(=O)c(O)c2C1=O ; CN1CCn2c(C3CCCN3C(=O)CSc3ccccc3Cl)nc(=O)c(O)c2C1=O ; CN1CCn2c(C3CCCN3C(=O)COc3ccccc3Cl)nc(=O)c(O)c2C1=O ; CN1CCn2c(C3CCCN3C(=O)COc3ccccc3Cl)nc(=O)c(O)c2C1=O ; "and"]

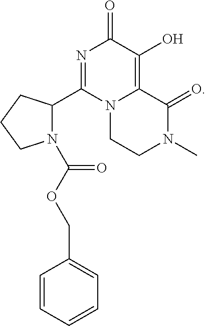 [SMILES: CN1CCn2c(C3CCCN3C(=O)OCc3ccccc3)nc(=O)c(O)c2C1=O]

14. A pharmaceutical composition comprising a therapeutically effective amount of the compound of claim 1, and a pharmaceutically acceptable carrier.

15. A method of treating a viral infection in a subject in need thereof, the method comprising administering to the subject an effective amount of the compound of claim 1.

16. The method of claim 13, wherein the subject is a mammal.

17. The method of claim 14, wherein the mammal is a human.

18. The method of claim 13, wherein the effective amount is a therapeutically effective amount.

19. The method of claim 13, wherein the viral infection is influenza.

20. A kit comprising the compound of claim 1, and one or more of:
(a) an antiviral agent;
(b) an immunity booster;
(c) instructions for administering the compound in connection with treating a viral infection;
(d) instructions for administering the compound in connection with reducing the risk of viral infection; and
(e) instructions for treating a viral infection.
